(19) United States
(12) Patent Application Publication
(10) Pub. No.: US 2015/0164907 A1
(43) Pub. Date: Jun. 18, 2015

(54) COMPOSITIONS AND METHODS FOR TREATING DISEASE

(71) Applicants: Lambertus J.M.W. OEHLEN, Westbury, NY (US); Itzhak D. GOLDBERG, Englewood, NJ (US)

(72) Inventors: Lambertus J.M.W. OEHLEN, Westbury, NY (US); Itzhak D. GOLDBERG, Englewood, NJ (US)

(73) Assignee: ANGION BIOMEDICA CORP., Uniondale, NY (US)

(21) Appl. No.: 14/542,974

(22) Filed: Nov. 17, 2014

Related U.S. Application Data
(60) Provisional application No. 61/905,252, filed on Nov. 17, 2013.

(51) Int. Cl. A61K 31/5377; A61K 31/4745; A61K 45/06; A61K 31/203

(52) U.S. Cl. CPC A61K 31/5377; A61K 31/203; A61K 31/4745; A61K 45/06

(57) ABSTRACT

Methods are provided for treating fibrotic diseases and conditions or cancer and other dysproliferative diseases by administering to a subject in need there of a therapeutically effective amount of a synergistic composition comprising a phosphatidylinositol 3-kinase inhibitor and a retinoid, optionally including a CYP26 inhibitor, wherein the therapeutically effective amount suppresses fibrosis or the growth of dysproliferative cells in vivo. Compositions comprising a combination of a phosphatidylinositol 3-kinase inhibitor and a retinoid, optionally in combination with a CYP26 inhibitor, are also described.

### BACKGROUND OF THE INVENTION

Numerous diseases and conditions responsible for significant morbidity as well as mortality have as an underlying disease mechanism the inappropriate or excessive production of fibrous connective tissue, a process generally known as fibrosis. Such diseases and conditions include by way of non-limiting examples, fibrotic liver disease, cirrhosis, cardiac fibrosis and lung fibrosis including idiopathic pulmonary fibrosis. In addition to these, numerous other conditions and diseases exhibit a fibrotic component, including but not limited to hepatic ischemia-reperfusion injury, cerebral infarction, chronic obstructive pulmonary diseases including emphysema, pancreatic fibrosis, ischemic heart disease, heart failure and renal disease including renal fibrosis. These conditions and diseases extract a major toll on the healths of afflicted individuals, and on the health care system. Furthermore, fibrosis is among the conditions that can affect the appearance and elastic properties of the skin, and conditions such as wrinkling, sagging, thickening, thinning, and other adverse features of photoaging and chronoaging that while not life threatening are desirous of being ameliorated. Diseases and other conditions that affect the skin such as psoriasis, ichthyosis, and acne are also in need of effective treatments.

Furthermore, dysproliferative diseases including cancer are also major diseases with a significant impact to the patients as well as the health care system.

Means to affect the onset or progression of such conditions and diseases would be highly desirable.

### SUMMARY OF THE INVENTION

In one embodiment, a method is provided for treating fibrotic conditions and diseases as well as cancer and other dysproliferative diseases comprising administering to a subject in need there of a therapeutically effective amount of a synergistic composition comprising a phosphatidylinositol 3-kinase inhibitor and a retinoid, optionally in further combination with a CYP26 inhibitor, wherein said therapeutically effective amount suppresses fibrosis or growth of dysproliferative cells in vivo. A synergistic composition provides benefit to the subject greater than the effect of any component separately or the sum of their effects when administered in the absence of the other(s). The phosphatidylinositol 3-kinase inhibitor (PI3Ki) and a retinoid and optionally including the CYP26 inhibitor can be administered in the same composition, or separately, provided that the synergistic benefit to the patient or subject of the two or optionally three components is achieved. Routes of administration such as topical, parenteral and oral are among non-limiting examples. In one embodiment, each component may be given by a different route of administration. In one embodiment, a dosage form comprising the two or optionally three components are administered at the same time by the same route preferably in a single formulation.

In another embodiment, the invention herein is directed to synergistic compositions and methods of their use for the treatment of fibrosis, cancer and other dysproliferative diseases. In one embodiment, a therapeutically effective amount of a synergistic combined preparation is provided comprising a PI3Ki or a pharmaceutically acceptable salt or prodrug form thereof; a retinoid, or a pharmaceutically acceptable salt or prodrug thereof; and optionally a CYP26 inhibitor, or a pharmaceutically acceptable salt or prodrug thereof; wherein said therapeutically effective amount suppresses fibrosis or the growth of dysproliferative cells in vivo.

As noted above, the aforementioned pharmaceutical compositions have anti-fibrotic activities and thus are useful for the prevention, treatment or lessening of the severity of a condition or disease associated with or characterized by increased, excessive or inappropriate fibrosis. In another embodiment, the compositions and pharmaceutical formulations have anti-dysproliferative activities and thus are useful for the prevention, treatment or lessening of the severity of a condition or disease associated with or characterized by increased, excessive or inappropriate proliferation, such as cancer.

In another aspect, the invention provides methods for the use of any of the compositions disclosed herein for treating or lessening the severity of a disease or condition associated with inappropriate fibrosis. In certain embodiments, the method is for treating or lessening the severity of a disease or condition selected from fibrotic liver disease, cirrhosis, cardiac fibrosis and lung fibrosis including idiopathic pulmonary fibrosis; hepatic ischemia-reperfusion injury, cerebral infarction, chronic obstructive pulmonary diseases including emphysema, pancreatic fibrosis, ischemic heart disease, heart failure and renal disease including renal fibrosis, fibrotic liver disease, hepatic ischemia-reperfusion injury, cerebral infarction, ischemic heart disease, and renal disease or lung (pulmonary) fibrosis. In certain embodiments, the method is for treating or lessening the severity of a disease or condition selected from liver fibrosis associated with hepatitis C, hepatitis B, delta hepatitis, chronic alcoholism, non-alcoholic steatohepatitis, extrahepatic obstructions (stones in the bile duct), cholangiopathies (primary biliary cirrhosis and sclerosing cholangitis), autoimmune liver disease, and inherited metabolic disorders (Wilson's disease, hemochromatosis, and alpha-1 antitrypsin deficiency); damaged and/or ischemic organs, transplants or grafts; ischemiareperfusion injury; stroke; cerebrovascular disease; myocardial ischemia; atherosclerosis; renal failure; renal fibrosis or idiopathic pulmonary fibrosis. In certain exemplary embodiments, the method is for the treatment of wounds for acceleration of healing; vascularization of a damaged and/or ischemic organ, transplant or graft; amelioration of ischemiareperfusion injury in the brain, heart, liver, kidney, and other tissues and organs; normalization of myocardial perfusion as a consequence of chronic cardiac ischemia or myocardial infarction; development or augmentation of collateral vessel development after vascular occlusion or to ischemic tissues or organs; fibrotic diseases; hepatic disease including fibrosis and cirrhosis; lung fibrosis; radiocontrast nephropathy; fibrosis secondary to renal obstruction; renal trauma and transplantation; renal failure secondary to chronic diabetes and/or hypertension; amytrophic lateral sclerosis, muscular dystrophy, scleroderma, chronic obstructive pulmonary disease, emphysema, diabetes mellitus, multiple sclerosis, trauma to the central nervous system, and hereditary neurodegenerative disorders including the leukodystrophies such as metachromatic leukodystrophy, Refsum's disease, adrenoleukodystrophy, Krabbe's disease, phenylketonuria, Canavan disease, Pelizaeus-Merzbacher disease and Alexander's disease.

In other embodiments, compositions of the invention are useful in the treatment of dysproliferative diseases including cancer, psoriasis, rheumatoid arthritis, and other inflammatory joint and skin diseases. In one embodiment, the compounds are useful in the treatment of prostate cancer. In one embodiment, the compounds are useful in the treatment of breast cancer.

In another embodiment, compositions of the invention are useful for prevention and treatment of other cancerous and precancerous conditions, including, for example, premalignant and malignant hyperproliferative diseases such as cancers of the breast, skin, prostate, colon, bladder, cervix, uterus, stomach, lung, esophagus, blood and lymphatic system, larynx, oral cavity, metaplasias, dysplasias, neoplasias, leukoplakias and papillomas of the mucous membranes, and in the treatment of Kaposi's sarcoma. In addition, inventive compounds can also be used as agents to treat diseases of the eye, including, for example, proliferative vitreoretinopathy, retinal detachment, corneopathies such as dry eye, as well as in the treatment and prevention of various cardiovascular diseases, including, without limitation, diseases associated with lipid metabolism such as dyslipidemias, prevention of post-angioplasty restenosis and as an agent to increase the level of circulation tissue plasminogen activator. Other uses include the prevention and treatment of conditions and diseases associated with human papilloma virus (HPV), including warts, various inflammatory diseases such as pulmonary fibrosis, ileitis, colitis and Crohn's disease, neurodegenerative diseases such as Alzheimer's disease, Parkinson's disease and stroke, improper pituitary function, including insufficient production of growth hormone, modulation of apoptosis, including both the induction of apoptosis, restoration of hair growth, including combination therapies with the present compounds and other agents such as minoxidil, diseases associated with the immune systems, including use of the present compounds as immunosuppressant and immunostimulants, modulation of organ transplant rejection and facilitation of wound healing, including modulation of chelosis. Compounds are also useful in treating type II non-insulin dependent diabetes mellitus (NIDDM).

The present invention is also directed to treatment of non-malignant tumors and other disorders involving inappropriate cell or tissue growth by administering a therapeutically effective amount of a composition of the invention. For example, it is contemplated that the invention is useful for the treatment of arteriovenous (AV) malformations, particularly in intracranial sites. The invention may also be used to treat psoriasis, a dermatologic condition that is characterized by inflammation and vascular proliferation; benign prostatic hypertrophy, a condition associated with inflammation and possibly vascular proliferation; and cutaneous fungal infections. Treatment of other hyperproliferative disorders is also contemplated. The agents may also be used topically to remove warts, birthmarks, moles, nevi, skin tags, lipomas, angiomas including hemangiomas, and other cutaneous lesions for cosmetic or other purposes.

In other embodiments, the invention is also directed to treatment of conditions and diseases that affect the skin. Skin is subject to deterioration through dermatological disorders, environmental abuse such as wind, air conditioning, central heating, or through the normal ageing process (chronoaging), which may be accelerated by exposure of skin to sun (photoaging). In recent years the demand for cosmetic methods for improving the appearance and condition and, in particular, for reversing, reducing or preventing the visible signs of wrinkled, aged and/or photodamaged skin has grown enormously. Consumers are increasingly seeking “anti-ageing” products that reverse, treat or delay the visible signs of chronoaging and photoaging skin such as wrinkles, lines, sagging, hyperpigmentation and age spots. Compositions and pharmaceutical formulation comprising a retinoid and a CYP26 inhibitor are useful for such conditions, in particular when one or both are administered via the topical route, concurrently or at different times.

In another embodiment, compounds of the invention as well as compositions and formulations thereof are therapeutically beneficial when administered at a time after the onset of the acute disease or acute condition or time of injury. In certain instances administration at least 3 hours after onset is beneficial. In other embodiments administration at least 24 hours after onset is beneficial. In certain other embodiments administration at least 1-3 weeks after onset is beneficial. In other embodiments methods are provided for treating an acute disease or condition wherein compound is administered at a time after the onset or induction of the disease or condition. In other embodiments, temporal separation of the induction, onset, recurrence or recrudescence of a disease or injury, and the optimal effective response to an antifibrotic or antidysproliferative compound, provides guidance to the timing of administration of a compound of the invention or a composition of formulation thereof. In other embodiments, a disease, condition or injury can be prevented by prophylactic administration of a compound embodied herein prior to the injury, exposure, or other anticipated sustaining of pathology.

In another embodiment, synergistic compositions of the invention are useful for prevention and treatment of other cancerous and precancerous conditions, including, for example, premalignant and malignant hyperproliferative diseases such as cancers of the breast, ovary, germ cell, skin, prostate, colon, bladder, cervix, uterus, stomach, lung, esophagus, blood and lymphatic system, larynx, oral cavity, as well as metaplasias, dysplasias, neoplasias, leukoplakias and papillomas of the mucous membranes, and in the treatment of Kaposi's sarcoma.

In other embodiments, the methods and synergistic compositions of the invention are useful in the treatment of dysproliferative diseases including cancer, psoriasis, rheumatoid arthritis, and other inflammatory joint and skin diseases. In one embodiment, the compositions are useful in the treatment of prostate cancer.

Furthermore, diseases and other conditions also affect the skin that the compositions embodied herein can benefit. Diseases including acne and psoriasis are examples. Ichthyosis refers to a group of skin disorders characterized by the presence of excessive amounts of dry surface scales. This disease manifests itself within the first year of life and is clinically characterized by thickened dry, scaly skin, keratosis pilaris, tightening and cracking of the skin and many white fish-like scales. There are common and rare forms including inherited and acquired forms of ichthyosis, and an estimated total of about 1.24 million afflicted individuals in the US population. The compositions of the invention are also directed to the treatment of the various types of ichthyoses.

Compound GDC-0941 is a potent and selective inhibitor of phosphatidylinositol 3-kinase (PI3K) (Folkes A J et al. The identification of 2-(1H-indazol-4-yl)-6-(4-methanesulfonyl-piperazin-1-ylmethyl)-4-morpholin-4-yl-thieno[3,2-d]pyrimidine (GDC-0941) as a potent, selective, orally bioavailable inhibitor of class I PI3 kinase for the treatment of cancer. J. Med. Chem. 2008 Sep. 25; 51(18):5522-32), and compound NVP-BEZ235 has been described to potently inhibit both phosphatidylinositol 3-kinase and mammalian target of rapamycin (mTOR) (Maira S M, et al., Targeting PI3K in cancer: any good news? Identification and characterization of NVP-BEZ235, a new orally available dual phosphatidylinositol 3-kinasemammalian target of rapamycin inhibitor with potent in vivo antitumor activity. Mol. Cancer Ther. 2008 July; 7(7):1851-63). Other PI3Ki include BKM120, PX866, BAY 80-6946, GDC-0032, GSK 2363771, IP-145 and CAL-101, described in Martini et al., 2013, Targeting PI3K in cancer: any good news?, Frontiers in Oncology 2013; 3(Article 108), 1-9. Other examples of PI3Ki are described in Owonikoko T K et al., Targeting the PI3K/AKT/mTOR Pathway: Biomarkers of Success and Tribulation; ASCO Educational Book 2003; e395-e401. The invention is not limited to any particular PI3Ki and any described herein or known in the art, at the research, clinical development or approved medicine stages are embraced herein.

Retinoids useful for the methods and compositions of the invention include, but are not limited to, retinol, 9-cis retinoic acid, 13-cis retinoic acid, retinyl esters, all-trans retinoic acid (ATRA), and fenretinide and other atypical retinoids. Retinol ((2E,4E,6E,8E)-3,7-dimethyl-9-(2,6,6-trimethylcyclohex-1-enyl)nona-2,4,6,8-tetraen-1-ol) is one of the animal forms of vitamin A. It is a diterpenoid and an alcohol. It is convertible to other forms of vitamin A, and the retinyl ester derivative of the alcohol serves as the storage form of the vitamin in animals. Commercial production of retinol typically requires retinal synthesis through reduction of a pentadiene derivative and subsequent acidificationhydrolysis of the resulting isomer to produce retinol. Pure retinol is extremely sensitive to oxidization and is prepared and transported at low temperatures and oxygen free atmospheres. When prepared as a dietary supplement, retinol is stabilized as the ester derivatives retinyl acetate or retinyl palmitate. Synthetic retinol is marketed under the following trade names: Acon, Afaxin, Agiolan, Alphalin, Anatola, Aoral, Apexol, Apostavit, Atav, Avibon, Avita, Avitol, Axerol, Dohyfral A, Epiteliol, Nio-A-Let, Prepalin, Testavol, Vaflol, Vi-Alpha, Vitpex, Vogan, and Vogan-Neu. 9-cis-retinoic acid is also called alitretinoin. 13-cis retinoic acid is also called isotretinoin, marketed as Accutane. ATRA is also called tretinoin and is marketed under the names Aberela, Airol, Renova, Atralin, Retin-A, Retino-A, Avita, Retacnyl, Refissa, ReTrieve, or Stieva-A). Atypical retinoids include but are not limited to fenretinide which is 4-hydroxy(phenyl)retinamide, abbreviated 4-HPR; and 6-[3-(1-adamantyl)-4-hydroxyphenyl]-2-naphthalene carboxylic acid (AHPN, CD437). Any and all of the foregoing retinoids are embraced by the teachings herein.

The invention is directed to a combination of the foregoing mentioned PI3K inhibitor, a retinoid, and optionally an agent or compound that inhibits the activity of cytochrome P450 (CYP) enzymes. In a further embodiment, this component of the composition embodied herein inhibits CYP26. In a further embodiment, the component embodied herein inhibits CYP17. In a further embodiment, the compositions embodied herein inhibit CYP19. In a further embodiment, the compositions embodied herein inhibit both CYP26 and CYP17, and may be referred to as dual inhibitors or dual CYP26 and CYP17 inhibitors. In a further embodiment, the compositions embodied herein inhibit both CYP26 and CYP19, and may be referred to as dual inhibitors or dual CYP26 and CYP19 inhibitors.

In one embodiment, the CYP26 inhibitor is selected from talarozole or rambazole.

In another embodiment, the CYP26 inhibitor is a compound of formula (I)

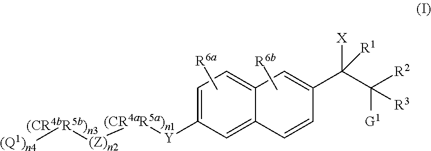

wherein G¹, R¹, R², R³, X, R^{6a}, R^{6b}, Y, R^{5a}, R^{4a}, Z, R^{5b}, R^{4b}, Q¹, n1, n2, n3 and n4 are as described generally and in classes and subclasses herein, tautomers thereof, Z and E isomers thereof, syn and anti isomers thereof, optically pure isomers thereof, pharmaceutically acceptable salts thereof, and pharmaceutical compositions thereof. Such CYP 26 inhibitors are described in WO2011016863 and US20120190716, which are incorporated herein by reference in their entireties.

In another embodiment, the CYP26 inhibitor is a compound of formula (I-A)

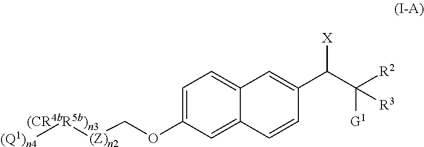

wherein G¹, R², R³, X, R^{4b}, R^{5b}, Z, Q¹, n2, n3 and n4 are as described generally and in classes and subclasses herein, tautomers thereof, Z and E isomers thereof, syn and anti isomers thereof, optically pure isomers thereof, pharmaceutically acceptable salts thereof, and pharmaceutical compositions thereof.

In another embodiment, the CYP26 inhibitor is a compound of formula (I-B)

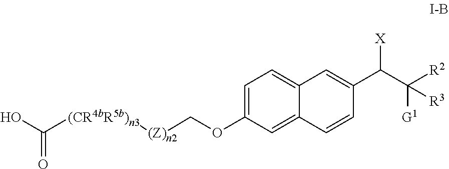

wherein G¹, R², R³, X, R^{4b}, R^{5b}, Z, n2 and n3 are as described generally and in classes and subclasses herein, tautomers thereof, Z and E isomers thereof, optically pure isomers thereof, and pharmaceutical compositions thereof.

In another embodiment, the CYP26 inhibitor is a compound of formula (II)

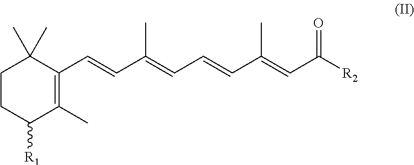

wherein R₁ and R₂ are as described generally and in classes and subclasses herein, tautomers thereof, Z and E isomers thereof, optically pure isomers thereof, and pharmaceutical compositions thereof.

In another embodiment, the CYP26 inhibitor is among those described and embraced in WO2011153192 and US20130102643, incorporated herein by reference in their entireties. By way of non-limiting example, the CYP26 inhibitor is a compound of formula (III):

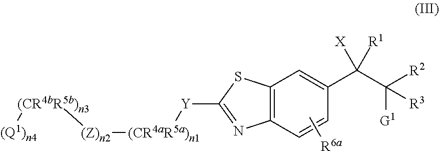

or an E or Z isomer thereof, syn or anti isomer thereof, an optically pure isomer thereof, or pharmaceutically acceptable salt thereof, wherein:
X is an unsaturated heterocycle selected from pyrrolyl, pyrazolyl, imidazolyl, triazolyl, benzimidazolyl, benzotriazolyl, tetrazolyl, thiazole, 3-pyridinyl or 4-pyridinyl, any of which is optionally substituted with one or more independent R⁶⁶ substituents;
R¹ is hydrogen, C₀₋₆alkyl, —OR⁷, —SR⁷, or —NR⁷R⁸;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, mono C₁₋₆alkylaminocarbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR⁷⁷—SO₂NR⁷¹R⁸¹ or —CONR⁷¹R⁸¹ substituents;
G¹ is hydrogen, C₀₋₁₀alkyl, C₂₋₁₀alkenyl, C₂₋₁₀alkynyl, —OR⁷², —SR⁷², —NR⁷²R⁸²(R⁹)ₙ₅, or G¹ and R³ taken together with the carbon atom to which they are attached forma 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Y is a bond, hydrogen, halogen, an oxygen atom, sulfur atom, —CN, R⁷⁴, —C(═NR⁷⁴)NR⁷⁵R⁷⁶, —C(═NR⁷⁴)OR⁷⁵—, —COR⁷⁴, —R⁷⁴COR⁷⁵, —CR⁷⁴R⁷⁵COR⁷⁶, —COOR⁷⁴, —R⁷⁴COOR⁷⁵, —CR⁷⁴R⁷⁵COOR⁷⁶, —CONR⁷⁴R⁷⁵, —CR⁷⁴R⁷⁵CONR⁷⁴R⁷⁵, —NR⁷⁴COOR⁷⁵, —NR⁷⁴COR⁷⁵, NR⁷⁴CONR⁷⁵R⁷⁶, —SO₂CR⁷⁴R⁷⁵, —SO₂NR⁷⁴R⁷⁵, —NR⁷⁴SO₂NR⁷⁵R⁷⁶, —NR^{6b}CO—, —NR^{6b}R⁷⁵, aryl or heteroaryl, any of which is optionally substituted with one or more independent halo, cyano, hydroxy, nitro, R⁶⁸, —SO₂N^{R6}R⁸⁶ or —NR⁷⁶R⁸⁶ substituents;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, or -alkenylhetaryl-, any of which is optionally substituted with one or more independent halo, cyano, hydroxy, nitro, R⁶⁸, C₁₋₁₀alkoxy, —COOR⁷⁴, —(C═O)N(R⁷⁴)—, —NR⁷⁴CONR⁷⁴R⁷⁵, —NR⁷⁴COOR⁷⁵, —SO₂N^{R6}R⁸⁶, —NR⁷⁴SO₂NR⁷⁴R⁷⁵, or —NR⁷⁶R⁸⁶ substituents;
Q¹ is C₀₋₆alkyl, cycloC₃₋₈alkyl, bridged bicycloalkyl, —OR⁷⁵, —COR⁷⁴, —NR⁷⁵R⁸⁵(R⁹⁵)ₙ₆, —CO₂R⁷⁵, —CONR⁷⁵R⁸⁵, —(C═S)OR⁷⁵, —(C═O)SR⁷⁵, —NO₂, —CN, halo, —S(O)ₙ₆R⁷⁵, —SO₂NR⁷⁵R⁸⁵, —NR⁷⁵(C═NR⁷⁷⁵)NR⁷⁷⁷⁵R⁸⁵, —NR⁷⁵(C═NR⁷⁷⁵)OR⁷⁷⁷⁵, —NR⁷⁵(C═NR⁷⁷⁵)SR⁷⁷⁷⁵, —O(C═O)OR⁷⁵, —O(C═O)NR⁷⁵R⁸⁵, —O(C═O)SR⁷⁵, —S(C═O)OR⁷⁵, —S(C═O)NR⁷⁵R⁸⁵, —S(C═O)SR⁷⁵, —NR⁷⁵(C═O)NR⁷⁷⁵R⁸⁵, or —NR⁷⁵(C═S)NR⁷⁷⁵R⁸⁵; in the case of —NR⁷⁵R⁸⁵(R⁹⁵)ₙ₆, R⁷⁵ and R⁸⁵ taken together with the nitrogen atom to which they are attached form a 3-10 membered heterocyclic saturated ring; or heterocyclic unsaturated ring, any of which is optionally substituted with one or more independent halo, cyano, hydroxy, nitro, C₁₋₁₀alkoxy, —SO₂N^{R6}R⁸⁶ or —NR⁷⁶R⁸⁶ substituents;
R^{4a}, R^{4b}, R^{4c}, R^{5a}, R^{5b} and R^{5c} are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkyl_{C1-10}alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono(C₁₋₆alkyl)amino C₁₋₆alkyl, di(C₁₋₆alkyl)aminoC₁₋₆alkyl, mono(aryl)amino C₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or R^{4a} with R^{5a}, or R^{4b} with R^{5b}, or R^{4c} with R^{5c}, taken together with the respective carbon atom to which they are attached, form a carbonyl or 3-10 membered saturated or unsaturated monocyclic or polycyclic ring, wherein said ring is optionally substituted with R⁶⁹; or R^{4a} with R^{5a}, or R^{4b} with R^{5b}, or R^{4c} with R^{5c}, taken together with the respective carbon atom to which they are attached, form a 3-10 membered saturated or unsaturated monoheterocyclic or polyheterocyclic ring, wherein said ring is optionally substituted with R⁶⁹;
R^{6a}, R^{6b}, R⁶⁶, R⁶⁷, R⁶⁸, and R⁶⁹ are each independently hydrogen, halo, —OR⁷⁷—SH, —NR⁷⁷R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthio C₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)amino C₁₋₆alkyl, di(C₁₋₆alkyl)aminoC₁₋₆alkyl, mono(aryl)amino C₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered mono or polycyclic saturated ring, mono or polycyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R⁸⁵, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynmyl, C₁₋₁₀alkylthioC₁₋₁₀alkyl, C₁₋₁₀alkylthio C₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl) or —N(C₀₋₄alkyl) (C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl) (C₀₋₄alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₄alkyl), —SO₂N(C₀₋₄ alkyl)(C₀₋₄alkyl) or —N(C₀₋₄alkyl) (C₀₋₄alkyl) substituents; and
n1, n2, n3, n4, n5, n6, and n7 are each independently equal to 0, 1 or 2.

In another embodiment, the CYP26 inhibitor is a compound of formula (IV):

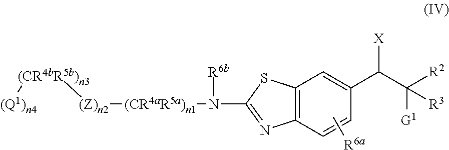

or an E or Z isomer thereof, syn or anti isomer thereof, an optically pure isomer thereof, or pharmaceutically acceptable salt thereof, and wherein all other substituents are as defined herein above.

In another embodiment, the CYP26 inhibitor is a compound selected from among compounds described in U.S. Pat. No. 7,265,143, which is incorporated herein by reference in its entirety.

In another embodiment, the CYP26 inhibitor is a compound of formula (IX):

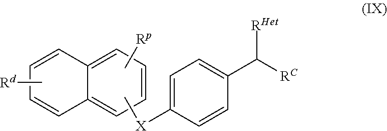

or an isomer, salt, solvate, chemically protected form or prodrug thereof,
wherein X is selected from O, S, NH or CH₂;
R^{d} and R^{P} are optional naphthyl group substituents;
R^{Het} is imidazolyl, triazolyl or pyridyl; and
R^{c} is C₁₋₄alkyl substituted by a group selected from: hydroxy, amino, amido, carboxy, C₁₋₇alkyl ester, C₅₋₇ aryl-C₁₋₂ alkyl ester, sulfonamino, sulfinamino, hydroxamino and tetrazolyl.

The compound of formula (IX) can be formula (IX-A) or formula (IX-B):

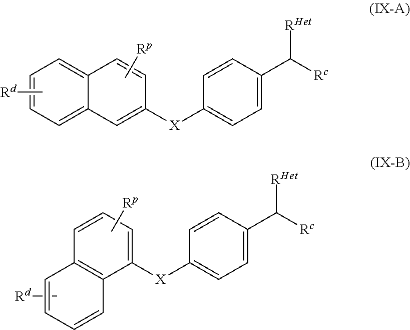

or an isomer, salt, solvate, chemically protected form or prodrug thereof,
wherein X is selected from O, S, NH or CH₂;
R^{Het} is imidazolyl, triazolyl or pyridyl; and
R^{c} is C₁₋₄alkyl substituted by a group selected from: hydroxy, amino, amido, carboxy, C₁₋₇alkyl ester, C₅₋₇ aryl-C₁₋₂ alkyl ester, sulfonamino, sulfinamino, hydroxamino and tetrazolyl; and
R^{d} and R^{P} are optionally selected from, but not limited to, C₁₋₇alkyl, C₃₋₂₀ heterocyclyl, C₅₋₂₀ aryl, halo, hydroxy, ether, nitro, cyano, acyl, ester, amido, amino, acylamido, ureido, acyloxy, thiol, thioether, sulfoxide, sulfonyl, thioamido, sulfonamino, sulfinamino and hydroxamino.

In another embodiment, the CYP26 inhibitor is a compound described in PCTGB2009001533, published as WO2009153566 on Dec. 23, 2009, incorporated herein by reference in its entirety.

In another embodiment, the CYP26 inhibitor is a compound described in Pautus et al., (2006), Synthesis and CYP26A1 inhibitory activity of 1-[benzofuran-2-yl-(4-alkyl/aryl-phenyl)-methyl]-1H-triazoles. Bioorg Med Chem. 2006 Jun. 1; 14(11):3643-53. Epub 2006 Feb. 3; Gomaa et al., (2011) Small molecule inhibitors of retinoic acid 4-hydroxylase (CYP26): synthesis and biological evaluation of imidazole methyl 3-(4-(aryl-2-ylamino)phenyl)propanoates, Med Chem. 2011 Apr. 28; 54(8):2778-91. Epub 2011 Mar. 23; and Gomaa et al., (2011) Synthesis and biological evaluation of 3-(1H-imidazol- and triazol-1-yl)-2,2-dimethyl-3-[4-(naphthalen-2-ylamino)phenyl]propyl derivatives as small molecule inhibitors of retinoic acid 4-hydroxylase (CYP26). J Med Chem. 2011 Oct. 13; 54(19):6803-11. Epub 2011 Sep. 8; all of which are incorporated herein by reference in their entireties.

The PI3Ki, a retinoid and the optional CYP26 inhibitor can be administered in the same composition, or separately, provided that the synergistic benefit to the patient of the two inhibitors is achieved. For example, one inhibitor may administered parenterally and the other orally. One or both may be administered topically. The frequency of administration may depend on the pharmacokinetics of each component such that the synergistic activity can be optimized without necessarily requiring simultaneous administration or administration by the same routes.

In another embodiment, synergistic compositions of the invention are useful for prevention and treatment of other cancerous and precancerous conditions, including, for example, premalignant and malignant hyperproliferative diseases such as cancers of the breast, ovary, germ cell, skin, prostate, colon, bladder, cervix, uterus, stomach, lung, esophagus, blood and lymphatic system, larynx, oral cavity, as well as metaplasias, dysplasias, neoplasias, leukoplakias and papillomas of the mucous membranes, and in the treatment of Kaposi's sarcoma.

In one embodiment, the compositions are useful in the treatment of breast cancer. In one embodiment the compositions are useful in the treatment of ovarian cancer.

In other embodiments, the synergistic compositions of the invention are useful in the treatment of dysproliferative diseases including cancer, psoriasis, rheumatoid arthritis, and other inflammatory joint and skin diseases. In one embodiment, the compositions are useful in the treatment of prostate cancer.

The present invention is also directed to treatment of non-malignant tumors and other disorders involving inappropriate cell or tissue growth by administering a therapeutically effective amount of a synergistic composition of the invention. For example, it is contemplated that the invention is useful for the treatment of arteriovenous (AV) malformations, particularly in intracranial sites. The invention may also be used to treat psoriasis, a dermatologic condition that is characterized by inflammation and vascular proliferation; benign prostatic hypertrophy, a condition associated with inflammation and possibly vascular proliferation; and cutaneous fungal infections. Treatment of other hyperproliferative disorders is also contemplated. The agents may also be used topically to remove warts, birthmarks, moles, nevi, skin tags, lipomas, angiomas including hemangiomas, and other cutaneous lesions for cosmetic or other purposes.

In another embodiment, the compositions described above comprising a PI3Ki and a retinoid, optionally including a CYP26 inhibitor are useful for the treatment of a dysproliferative disease. This, in another embodiment, a method is provided for treating a dysproliferative disease in a subject in need thereof comprising administering to the subject an effective amount of a composition comprising a PI3Ki, a retinoid and optionally a CYP26 inhibitor. In another embodiment, the retinoid is, by way of non-limiting example, selected from among those compounds and agents described herein above. In another embodiment, the CYP26 inhibitor is, by way of non-limiting example, a compound as described above. In another embodiment, a method is provided for treating dysproliferative diseases including cancer, psoriasis, rheumatoid arthritis, and other inflammatory joint, skin diseases and undesirable cosmetic features of the skin. In one embodiment, a method is provided for the treatment of prostate cancer. In one embodiment, a method is provided for the treatment of breast cancer.

In another embodiment, methods described here are provided for prevention and treatment of other cancerous and precancerous conditions, including, for example, premalignant and malignant hyperproliferative diseases such as cancers of the breast, skin, prostate, colon, bladder, cervix, uterus, stomach, lung, esophagus, blood and lymphatic system, larynx, oral cavity, metaplasias, dysplasias, neoplasias, leukoplakias and papillomas of the mucous membranes, and in the treatment of Kaposi's sarcoma. In addition, methods are provided to treat diseases of the eye, including, for example, proliferative vitreoretinopathy, retinal detachment, corneopathies such as dry eye, as well as in the treatment and prevention of various cardiovascular diseases, including, without limitation, diseases associated with lipid metabolism such as dyslipidemias, prevention of post-angioplasty restenosis and as an agent to increase the level of circulation tissue plasminogen activator. Other methods embodied herein include the prevention and treatment of conditions and diseases associated with human papilloma virus (HPV), including warts, various inflammatory diseases such as pulmonary fibrosis, ileitis, colitis and Crohn's disease, neurodegenerative diseases such as Alzheimer's disease, Parkinson's disease and stroke, improper pituitary function, including insufficient production of growth hormone, modulation of apoptosis, including both the induction of apoptosis, restoration of hair growth, including combination therapies with the present compositions and other agents such as minoxidil, diseases associated with the immune systems, including use of the present compositions as immunosuppressant and immunostimulants, modulation of organ transplant rejection and facilitation of wound healing, including modulation of chelosis. Synergistic compositions embodied herein are also useful in treating type II non-insulin dependent diabetes mellitus (NIDDM).

### BRIEF DESCRIPTION OF THE FIGURES

FIG. 1 shows the effect of GDC-0941 (A) or NVP-BEZ235 (B) with and without ATRA on proliferation of SK-BR3 breast cancer cells;

FIG. 2 shows the CI values from the experiment in FIGS. 1 A and B, respectively; and

FIG. 3 shows the CI values from a similar experiment using the cancer cell line UACC812.

### DEFINITIONS

Unless otherwise stated, the connections of compound name moieties are at the rightmost recited moiety. That is, the substituent name starts with a terminal moiety, continues with any bridging moieties, and ends with the connecting moiety. For example, hetarylthioC₁₋₄ alkyl has a heteroaryl group connected through a thio sulfur to a C₁₋₄alkyl that connects to the chemical species bearing the substituent.

As used herein, for example, “C₀₋₄alkyl” is used to mean an alkyl having 0-4 carbons—that is, 0, 1, 2, 3, or 4 carbons in a straight or branched configuration. An alkyl having no carbon is hydrogen when the alkyl is a terminal group. An alkyl having no carbon is a direct bond when the alkyl is a bridging (connecting) group.

In all embodiments of this invention, the term “alkyl” includes both branched and straight chain alkyl groups. Typical alkyl groups are methyl, ethyl, n-propyl, isopropyl (iPr), n-butyl, sec-butyl, isobutyl, tert-butyl, n-pentyl, isopentyl, n-hexyl, n-heptyl, isooctyl, nonyl, decyl, undecyl, dodecyl, tetradecyl, hexadecyl, octadecyl, eicosyl and the like.

The term “halo” or “halogen” refers to fluoro, chloro, bromo or iodo.

The term “haloalkyl” refers to an alkyl group substituted with one or more halo groups, for example chloromethyl, 2-bromoethyl, 3-iodopropyl, trifluoromethyl, perfluoropropyl, 8-chlorononyl and the like.

The term “cycloalkyl” or “saturated ring” refers to a cyclic aliphatic ring structure, optionally substituted with alkyl, hydroxy and halo, such as cyclopropyl, methylcyclopropyl, cyclobutyl, cyclopentyl, 2-hydroxycyclopentyl, cyclohexyl, 4-chlorocyclohexyl, cycloheptyl, cyclooctyl and the like.

The term “bicycloalkyl” refers to two cycloalkyl rings fused together and the term “bridged bicycloalkyl” refers to two rings joined together forming a bridged structure, for example bicyclo[3.2.1]octane, bicyclo[2.2.1]heptane, bicyclo[2.2.2]octane and the like.

The term “alkylcarbonyloxyalkyl” refers to an ester moiety, for example acetoxymethyl, n-butyryloxyethyl and the like.

The term “alkynylcarbonyl” refers to an alkynylke to functionality, for example propynoyl and the like.

The term “hydroxyalkyl” refers to an alkyl group substituted with one or more hydroxy groups, for example hydroxymethyl, 2,3-dihydroxybutyl and the like.

The term “alkylsulfonylalkyl” refers to an alkyl group substituted with an alkylsulfonyl moiety, for example mesylmethyl, isopropylsulfonylethyl and the like.

The term “alkylsulfonyl” refers to a sulfonyl moiety substituted with an alkyl group, for example mesyl, n-propylsulfonyl and the like.

The term “acetylaminoalkyl” refers to an alkyl group substituted with an amide moiety, for example acetylaminomethyl and the like.

The term “acetylaminoalkenyl” refers to an alkenyl group substituted with an amide moiety, for example 2-(acetylamino)vinyl and the like.

The term “alkenyl” refers to an ethylenically unsaturated hydrocarbon group, straight or branched chain, having 1 or 2 ethylenic bonds, for example vinyl, allyl, 1-butenyl, 2-butenyl, isopropenyl, 2-pentenyl and the like.

The term “haloalkenyl” refers to an alkenyl group substituted with one or more halo groups.

The term “unsaturated ring” refers to a substituted or unsubstituted “cycloalkenyl” or a phenyl group.

The term “cycloalkenyl” refers to a cyclic aliphatic ring structure, optionally substituted with alkyl, hydroxy and halo, having 1 or 2 ethylenic bonds such as methylcyclopropenyl, trifluoromethylcyclopropenyl, cyclopentenyl, cyclohexenyl, 1,4-cyclohexadienyl and the like.

The term “alkynyl” refers to an unsaturated hydrocarbon group, straight or branched, having 1 or 2 acetylenic bonds, for example ethynyl, propargyl and the like.

The term “haloalkynyl” refers to an alkynyl group substituted with one or more halo groups.

The term “alkylcarbonyl” refers to an alkylke to functionality, for example acetyl, n-butyryl and the like.

The term “alkenylcarbonyl” refers to an alkenylke to functionality, for example, propenoyl and the like.

The term “aryl” refers to phenyl or naphthyl which may be optionally substituted. Typical aryl groups include, but are not limited to, phenyl, 4-chlorophenyl, 4-fluorophenyl, 4-bromophenyl, 3-chlorophenyl, 3-fluorophenyl, 3-nitrophenyl, 3-(trifluoromethyl)phenyl, 2-methoxyphenyl, 2-methylphenyl, 3-methyphenyl, 4-methylphenyl, 4-ethylphenyl, 2-methyl-3methoxyphenyl, 2,4-dibromophenyl, 3,5-difluorophenyl, 3,5-dimethylphenyl, 2,4,6-trichlorophenyl, 4-methoxyphenyl, naphthyl, 2-chloronaphthyl, 2,4-dimethoxyphenyl, 4-(trifluoromethyl)phenyl and 2-iodo-4-methylphenyl.

The term “heterocyclic unsaturated ring” refers to a substituted or unsubstituted “heteroaryl” or a heteroaliphatic ring structure having 1 or 2 ethylenic bonds such as dihydropyridine, tetrahydropyridine, dihydropyrrole, dihydroimidazole and the like.

The terms “heteroaryl” or “hetaryl” refer to a substituted or unsubstituted 3-10 membered unsaturated ring containing one, two, three or four heteroatoms, preferably one or two heteroatoms independently selected from oxygen, nitrogen and sulfur or to a bicyclic unsaturated ring system containing up to 10 atoms including at least one heteroatom selected from oxygen, nitrogen and sulfur. Examples of heteroaryls include, but are not limited to, 2-pyridinyl (synonym: 2-pyridyl), 3-pyridinyl (synonym: 3-pyridyl) or 4-pyridinyl (synonym: 4-pyridyl), pyrazinyl, 2-, 4-, or 5-pyrimidinyl, pyridazinyl, triazolyl, tetrazolyl, imidazolyl, 2- or 3-thienyl, 2- or 3-furyl, pyrrolyl, oxazolyl, isoxazolyl, thiazolyl, isothiazolyl, oxadiazolyl, thiadiazolyl, quinolyl, isoquinolyl, benzimidazolyl, benzotriazolyl, benzofuranyl, and benzothienyl. The heterocyclic ring may be optionally substituted with up to two substituents.

The terms “aryl-alkyl” or “arylalkyl” are used to describe a group wherein the alkyl chain can be branched or straight chain with the aryl portion, as defined hereinbefore, forming a bridging portion of the aryl-alkyl moiety. Examples of aryl-alkyl groups include, but are not limited to, optionally substituted benzyl, phenethyl, phenpropyl and phenbutyl such as 4-chlorobenzyl, 2,4-dibromobenzyl, 2-methylbenzyl, 2-(3-fluorophenyl)ethyl, 2-(4-methylphenyl)ethyl, 2-(4-(trifluoromethyl)phenyl)ethyl, 2-(2-methoxyphenyl)ethyl, 2-(3-nitrophenyl)ethyl, 2-(2,4-dichlorophenyl)ethyl, 2-(3,5-dimethoxyphenyl)ethyl, 3-phenylpropyl, 3-(3-chlorophenyl)propyl, 3-(2-methylphenyl)propyl, 3-(4-methoxyphenyl)propyl, 3-(4-(trifluoromethyl)phenyl)propyl, 3-(2,4-dichlorophenyl)propyl, 4-phenylbutyl, 4-(4-chlorophenyl)butyl, 4-(2-methylphenyl)butyl, 4-(2,4-dichlorophenyl)butyl, 4-(2-methoxphenyl)butyl and 10-phenyldecyl.

The terms “aryl-cycloalkyl” or “arylcycloalkyl” are used to describe a group wherein the aryl group is attached to a cycloalkyl group, for example phenylcyclopentyl and the like.

The terms “aryl-alkenyl” or “arylalkenyl” are used to describe a group wherein the alkenyl chain can be branched or straight chain with the aryl portion, as defined hereinbefore, forming a bridging portion of the aralkenyl moiety, for example styryl (2-phenylvinyl), phenpropenyl and the like.

The terms “aryl-alkynyl” or “arylalkynyl” are used to describe a group wherein the alkynyl chain can be branched or straight chain with the aryl portion, as defined hereinbefore, forming a bridging portion of the aryl-alkynyl moiety, for example 3-phenyl-1-propynyl and the like.

The terms “aryl-oxy” or “aryloxy” are used to describe a terminal aryl group attached to a bridging oxygen atom. Typical aryl-oxy groups include phenoxy, 3,4-dichlorophenoxy and the like.

The terms “aryl-oxyalkyl” or “aryloxyalkyl” are used to describe a group wherein an alkyl group is substituted with an aryl-oxy group, for example pentafluorophenoxymethyl and the like.

The terms “hetaryl-oxy” or “heteroaryl-oxy” or “hetaryloxy” or “heteroaryloxy” are used to describe a terminal hetaryl group attached to a bridging oxygen atom. Typical hetaryl-oxy groups include 4,6-dimethoxypyrimidin-2-yloxy and the like.

The terms “hetarylalkyl” or “heteroarylalkyl” or “hetaryl-alkyl” or “heteroaryl-alkyl” are used to describe a group wherein the alkyl chain can be branched or straight chain with the heteroaryl portion, as defined hereinbefore, forming a bridging portion of the heteroaralkyl moiety, for example 3-furylmethyl, thienyl, furfuryl and the like.

The terms “hetarylalkenyl” or “heteroarylalkenyl” or “hetaryl-alkenyl” or “heteroaryl-alkenyl” are used to describe a group wherein the alkenyl chain can be branched or straight chain with the heteroaryl portion, as defined hereinbefore, forming a bridging portion of the heteroaralkenyl moiety, for example 3-(4-pyridyl)-1-propenyl.

The terms “hetarylalkynyl” or “heteroarylalkynyl” or “hetaryl-alkynyl” or “heteroaryl-alkynyl” are used to describe a group wherein the alkynyl chain can be branched or straight chain with the heteroaryl portion, as defined hereinbefore, forming a bridging portion of the heteroaralkynyl moiety, for example 4-(2-thienyl)-1-butyryl.

The term “heterocyclyl” or “heterocyclic saturated ring” refers to a substituted or unsubstituted 3-10 membered saturated ring containing one, two or three heteroatoms, preferably one or two heteroatoms independently selected from oxygen, nitrogen and sulfur or to a bicyclic ring system containing up to 10 atoms including at least one hetero atom selected from oxygen, nitrogen and sulfur wherein the ring containing the heteroatom is saturated. Examples of heterocyclyls include, but are not limited to, tetrahydrofuranyl, tetrahydrofuryl, pyrrolidinyl, piperidinyl, 4-pyranyl, tetrahydropyranyl, thiolanyl, morpholinyl, piperazinyl, dioxolanyl, dioxanyl, indolinyl and 5-methyl-6-chromanyl.

The term “monoheterocyclic” refers to a single heterocyclic ring structure, while “polyheterocyclic” refers to more than one ring fused together to form a heterocyclic structure.

The terms “heterocyclylalkyl” or “heterocyclyl-alkyl” are used to describe a group wherein the alkyl chain can be branched or straight chain with the heterocyclyl portion, as defined hereinabove, forming a bridging portion of the heterocyclylalkyl moiety, for example 3-piperidinylmethyl and the like.

The terms “heterocyclylalkenyl” or “heterocyclyl-alkenyl” are used to describe a group wherein the alkenyl chain can be branched or straight chain with the heterocyclyl portion, as defined hereinbefore, forming a bridging portion of the heterocyclylalkenyl moiety, for example 2-morpholinyl-1-propenyl.

The terms “heterocyclylalkynyl” or “heterocyclyl-alkynyl” are used to describe a group wherein the alkynyl chain can be branched or straight chain with the heterocyclyl portion, as defined hereinbefore, forming a bridging portion of the heterocyclylalkynyl moiety, for example 2-pyrrolidinyl-1-butyryl.

The term “carboxylalkyl” includes both branched and straight chain alkyl groups as defined hereinbefore attached to a carboxyl (—COOH) group.

The term “carboxylalkenyl” includes both branched and straight chain alkenyl groups as defined hereinbefore attached to a carboxyl (—COOH) group.

The term “carboxylalkynyl” includes both branched and straight chain alkynyl groups as defined hereinbefore attached to a carboxyl (—COOH) group.

The term “carboxylcycloalkyl” refers to a carboxyl (—COOH) group attached to a cyclic aliphatic ring structure as defined hereinbefore.

The term “carboxylcycloalkenyl” refers to a carboxyl (—COOH) group attached to a cyclic aliphatic ring structure having 1 or 2 ethylenic bonds as defined hereinbefore.

The terms “cycloalkylalkyl” or “cycloalkyl-alkyl” refer to a cycloalkyl group as defined hereinbefore attached to an alkyl group, for example cyclopropylmethyl, cyclohexylethyl and the like.

The terms “cycloalkylalkenyl” or “cycloalkyl-alkenyl” refer to a cycloalkyl group as defined hereinbefore attached to an alkenyl group, for example cyclohexylvinyl, cycloheptylallyl and the like.

The terms “cycloalkylalkynyl” or “cycloalkyl-alkynyl” refer to a cycloalkyl group as defined hereinbefore attached to an alkynyl group, for example cyclopropylpropargyl, 4-cyclopentyl-2-butynyl and the like.

The terms “cycloalkenylalkyl” or “cycloalkenyl-alkyl” refer to a cycloalkenyl group as defined hereinbefore attached to an alkyl group, for example 2(cyclopenten-1-yl)ethyl and the like.

The terms “cycloalkenylalkenyl” or “cycloalkenyl-alkenyl” refer to a cycloalkenyl group as defined hereinbefore attached to an alkenyl group, for example 1-(cyclohexen-3-yl)allyl and the like.

The terms “cycloalkenylalkynyl” or “cycloalkenyl-alkynyl” refer to a cycloalkenyl group as defined hereinbefore attached to an alkynyl group, for example 1-(cyclohexen-3-yl)propargyl and the like.

The term “carboxylcycloalkylalkyl” refers to a carboxyl (—COOH) group attached to the cycloalkyl ring portion of a cycloalkylalkyl group as defined hereinbefore.

The term “carboxylcycloalkylalkenyl” refers to a carboxyl (—COOH) group attached to the cycloalkyl ring portion of a cycloalkylalkenyl group as defined hereinbefore.

The term “carboxylcycloalkylalkynyl” refers to a carboxyl (—COOH) group attached to the cycloalkyl ring portion of a cycloalkylalkynyl group as defined hereinbefore.

The term “carboxylcycloalkenylalkyl” refers to a carboxyl (—COOH) group attached to the cycloalkenyl ring portion of a cycloalkenylalkyl group as defined hereinbefore.

The term “carboxylcycloalkenylalkenyl” refers to a carboxyl (—COOH) group attached to the cycloalkenyl ring portion of a cycloalkenylalkenyl group as defined hereinbefore.

The term “carboxylcycloalkenylalkynyl” refers to a carboxyl (—COOH) group attached to the cycloalkenyl ring portion of a cycloalkenylalkynyl group as defined hereinbefore.

The term “alkoxy” includes both branched and straight chain terminal alkyl groups attached to a bridging oxygen atom. Typical alkoxy groups include methoxy, ethoxy, n-propoxy, isopropoxy, tert-butoxy and the like.

The term “haloalkoxy” refers to an alkoxy group substituted with one or more halo groups, for example chloromethoxy, trifluoromethoxy, difluoromethoxy, perfluoroisobutoxy and the like.

The term “alkoxyalkoxyalkyl” refers to an alkyl group substituted with an alkoxy moiety which is in turn substituted with a second alkoxy moiety, for example methoxymethoxymethyl, isopropoxymethoxyethyl and the like.

The term “alkylthio” includes both branched and straight chain alkyl groups attached to a bridging sulfur atom, for example methylthio.

The term “haloalkylthio” refers to an alkylthio group substituted with one or more halo groups, for example trifluoromethylthio.

The term “alkoxyalkyl” refers to an alkyl group substituted with an alkoxy group, for example isopropoxymethyl.

The term “alkoxyalkenyl” refers to an alkenyl group substituted with an alkoxy group, for example 3-methoxyallyl.

The term “alkoxyalkynyl” refers to an alkynyl group substituted with an alkoxy group, for example 3-methoxypropargyl.

The term “alkoxycarbonylalkyl” refers to a straight chain or branched alkyl substituted with an alkoxycarbonyl, for example ethoxycarbonylmethyl, 2-(methoxycarbonyl)propyl and the like.

The term “alkoxycarbonylalkenyl” refers to a straight chain or branched alkenyl as defined hereinbefore substituted with an alkoxycarbonyl, for example 4-(ethoxycarbonyl)-2-butenyl and the like.

The term “alkoxycarbonylalkynyl” refers to a straight chain or branched alkynyl as defined hereinbefore substituted with an alkoxycarbonyl, for example 4-(ethoxycarbonyl)-2-butynyl and the like.

The term “haloalkoxyalkyl” refers to a straight chain or branched alkyl as defined hereinbefore substituted with a haloalkoxy, for example 2-chloroethoxymethyl, trifluoromethoxymethyl and the like.

The term “haloalkoxyalkenyl” refers to a straight chain or branched alkenyl as defined hereinbefore substituted with a haloalkoxy, for example 4-(chloromethoxy)-2-butenyl and the like.

The term “haloalkoxyalkynyl” refers to a straight chain or branched alkynyl as defined hereinbefore substituted with a haloalkoxy, for example 4-(2-fluoroethoxy)-2-butynyl and the like.

The term “alkylthioalkyl” refers to a straight chain or branched alkyl as defined hereinbefore substituted with an alkylthio group, for example methylthiomethyl, 3-(isobutylthio)heptyl and the like.

The term “alkylthioalkenyl” refers to a straight chain or branched alkenyl as defined hereinbefore substituted with an alkylthio group, for example 4-(methylthio)-2-butenyl and the like.

The term “alkylthioalkynyl” refers to a straight chain or branched alkynyl as defined hereinbefore substituted with an alkylthio group, for example 4-(ethylthio)-2-butynyl and the like.

The term “haloalkylthioalkyl” refers to a straight chain or branched alkyl as defined hereinbefore substituted with an haloalkylthio group, for example 2-chloroethylthiomethyl, trifluoromethylthiomethyl and the like.

The term “haloalkylthioalkenyl” refers to a straight chain or branched alkenyl as defined hereinbefore substituted with an haloalkylthio group, for example 4-(chloromethylthio)-2-butenyl and the like.

The term “haloalkylthioalkynyl” refers to a straight chain or branched alkynyl as defined hereinbefore substituted

The term “dialkoxyphosphorylalkyl” refers to two straight chain or branched alkoxy groups as defined hereinbefore attached to a pentavalent phosphorous atom, containing an oxo substituent, which is in turn attached to an alkyl, for example diethoxyphosphorylmethyl.

The term “oligomer” refers to a low-molecular weight polymer, whose number average molecular weight is typically less than about 5000 gmol, and whose degree of polymerization (average number of monomer units per chain) is greater than one and typically equal to or less than about 50.

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above Formula (I) is shown without a definitive stereochemistry at certain positions. The present invention includes all stereoisomers of Formula (I) and pharmaceutically acceptable salts thereof. Further, mixtures of stereoisomers as well as isolated specific stereoisomers are also included.

During the course of the synthetic procedures used to prepare such compounds, or in using racemization or epimerization procedures known to those skilled in the art, the products of such procedures can be a mixture of stereoisomers.

Within the enantiomers of the compounds, both the syn and anti isomers involving the X and G¹ substituent show activity. Furthermore, it is preferable that there be dual chiral centers at the X and G¹ attachment positions, if G¹ is not hydrogen.

The invention also encompasses a pharmaceutical composition that is comprised of a compound of Formula (I) in combination with a pharmaceutically acceptable carrier.

Such a composition is comprised of a pharmaceutically acceptable carrier, excipient or diluent, and a non-toxic therapeutically effective amount of a compound of Formula (I) as described above, or an E or Z isomer thereof, syn or anti isomer thereof, an optically pure isomer thereof, or a pharmaceutically acceptable salt thereof.

Moreover, within this embodiment, the invention encompasses a pharmaceutical composition for the treatment of disease by inhibiting the cytochrome retinoic; acid 4-hydroxylase enzyme (CYP26) or by inhibiting cytochrome P450 17alpha-hydroxylase C_{17,20}-lyase enzyme (CYP17), or in certain instances inhibiting both CYP26 and CYP17, comprising a pharmaceutically acceptable carrier and a non-toxic therapeutically effective amount of compound of Formula (I) as described above or a pharmaceutically acceptable salt thereof. In other embodiments, the invention encompasses pharmaceuticals compositions for the treatment of disease by inhibiting the cytochrome aromatase enzyme (CYP19). In other embodiments, pharmaceutical compositions are provided for inhibiting both CYP26 and CYP19, comprising a pharmaceutically acceptable carrier and a non-toxic therapeutically effective amount of compound of Formula (I) as described above or a pharmaceutically acceptable salt thereof.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium slats. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, formic, hydrochloric, maleic, phosphoric, sulfuric and tartaric acids. Particularly preferred are formic and hydrochloric acid.

The pharmaceutical compositions of the present invention comprise a compound represented by Formula (I) (or E or Z isomer thereof, syn or anti isomer thereof, an optically pure isomer thereof, or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester, which is cleaved in vivo to yield a compound of interest. Another example is an N-methyl derivative of a compound, which is susceptible to oxidative metabolism resulting in N-demethylation. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

The term “tautomerization” refers to the phenomenon wherein a proton of one atom of a molecule shifts to another atom. See, Jerry March, Advanced Organic Chemistry: Reactions, Mechanisms and Structures, Fourth Edition, John Wiley & Sons, pages 69-74 (1992). The term “tautomer” as used herein, refers to the compounds produced by the proton shift. Thus, the present invention encompasses the tautomeric moieties like pyrazoles, pyridones and enols, etc.

The term “geometrical isomers” refers to cis-trans isomerism, syn-anti or EZ isomerism based on the Cahn-Ingold-Prelog system. See March's Advanced Organic Chemistry: Reactions, Mechanisms and Structures, Sixth Edition, Wiley-Interscience, pages 182-195 (2007). The term “geometrical isomers” as used herein, refers to compounds having double bond with an E or Z configuration or cis-trans isomers of monocyclic or fused ring systems.

By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, as detailed herein, certain exemplary oxygen protecting groups are utilized. These oxygen protecting groups include, but are not limited to methyl ethers, substituted methyl ethers (e.g., MOM (methoxymethyl ether), MTM (methylthiomethyl ether), BOM (benzyloxymethyl ether), PMBM or MPM (p-methoxybenzyloxymethyl ether), to name a few), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether), to name a few), esters (e.g., formate, acetate, benzoate (Bz), trifluoroacetate, dichloroacetate, to name a few), carbonates, cyclic acetals and ketals. In certain other exemplary embodiments, nitrogen protecting groups are utilized. These nitrogen protecting groups include, but are not limited to, carbamates (including methyl, ethyl and substituted ethyl carbamates (e.g., Troc), to name a few) amides, cyclic imide derivatives, N-Alkyl and N-Aryl amines, imine derivatives, and enamine derivatives, to name a few. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

As used herein, the term “isolated” when applied to the compounds of the present invention, refers to such compounds that are (i) separated from at least some components with which they are associated in nature or when they are made and/or (ii) produced, prepared or manufactured by the hand of man.

As used herein the term “biological sample” includes, without limitation, cell cultures or extracts thereof; biopsied material obtained from an animal (e.g., mammal) or extracts thereof; and blood, saliva, urine, feces, semen, tears, or other body fluids or extracts thereof; or purified versions thereof. For example, the term “biological sample” refers to any solid or fluid sample obtained from, excreted by or secreted by any living organism, including single-celled micro organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). The biological sample can be in any form, including a solid material such as a tissue, cells, a cell pellet, a cell extract, cell homogenates, or cell fractions; or a biopsy, or a biological fluid. The biological fluid may be obtained from any site (e.g. blood, saliva (or a mouth wash containing buccal cells), tears, plasma, serum, urine, bile, seminal fluid, cerebrospinal fluid, amniotic fluid, peritoneal fluid, and pleural fluid, or cells therefrom, aqueous or vitreous humor, or any bodily secretion), a transudate, an exudate (e.g. fluid obtained from an abscess or any other site of infection or inflammation), or fluid obtained from a joint (e.g. a normal joint or a joint affected by disease such as rheumatoid arthritis, osteoarthritis, gout or septic arthritis). The biological sample can be obtained from any organ or tissue (including a biopsy or autopsy specimen) or may comprise cells (whether primary cells or cultured cells) or medium conditioned by any cell, tissue or organ. Biological samples may also include sections of tissues such as frozen sections taken for histological purposes. Biological samples also include mixtures of biological molecules including proteins, lipids, carbohydrates and nucleic acids generated by partial or complete fractionation of cell or tissue homogenates. Although the sample is preferably taken from a human subject, biological samples may be from any animal, plant, bacteria, virus, yeast, etc. The term animal, as used herein, refers to humans as well as non-human animals, at any stage of development, including, for example, mammals, birds, reptiles, amphibians, fish, worms and single cells. Cell cultures and live tissue samples are considered to be pluralities of animals. In certain exemplary embodiments, the non-human animal is a mammal (e.g., a rodent, a mouse, a rat, a rabbit, a monkey, a dog, a cat, a sheep, cattle, a primate, or a pig). An animal may be a transgenic animal or a human clone. If desired, the biological sample may be subjected to preliminary processing, including preliminary separation techniques.

### DETAILED DESCRIPTION OF CERTAIN PREFERRED EMBODIMENTS

The invention herein is directed in one embodiment to compositions and methods for treating fibrotic conditions and diseases, and cancer and other dysproliferative diseases. The method comprises administering to a subject in need there of a therapeutically effective amount of a synergistic composition comprising a PI3K inhibitor or a pharmaceutically acceptable salt or prodrug there, and a retinoid, or a pharmaceutically acceptable salt or prodrug thereof; and optionally including a CYP26 inhibitor, or a pharmaceutically acceptable salt or prodrug thereof; wherein said therapeutically effective amount suppresses fibrosis or the growth of dysproliferative cells in vivo. A synergistic composition provides benefit to the subject which benefit is greater than the effect of either component separately or the sum of their effects when administered in the absence of the other. The PI3Ki, retinoid and the optional CYP26 inhibitor can be administered in the same composition, or separately, provided that the synergistic benefit to the patient of the two or three inhibitors is achieved. For example, in the case of a combination of a PI3Ki and a retinoid, one compound may administered parenterally and the other orally; or one or both topically. In the case of three compounds, each may be independently administered by any route. The frequency of administration may depend on the pharmacokinetics of each component such that the synergistic activity can be optimized without necessarily requiring simultaneous administration or administration by the same routes.

In another embodiment, the methods of treatment and synergistic compositions of the invention are useful for prevention and treatment of other cancerous and precancerous conditions, including, for example, premalignant and malignant hyperproliferative diseases such as cancers of the breast, ovary, germ cell, skin, prostate, colon, bladder, cervix, uterus, stomach, lung, esophagus, blood and lymphatic system, larynx, oral cavity, as well as metaplasias, dysplasias, neoplasias, leukoplakias and papillomas of the mucous membranes, and in the treatment of Kaposi's sarcoma.

In one embodiment, the methods and compositions are useful in the treatment of breast cancer. In one embodiment the methods and compositions are useful in the treatment of ovarian cancer. In another embodiment the methods and compositions are useful in the treatment of HER2 expressing cancers. In another embodiment the methods and compositions are useful in the treatment of HER2 expressing breast cancer. In another embodiment the methods and compositions are useful in the treatment of HER2 expressing ovarian cancer. In one embodiment the methods and compositions are useful in the treatment of triple-negative cancers.

In other embodiments, the synergistic compositions of the invention are useful in the treatment of dysproliferative diseases including cancer, psoriasis, rheumatoid arthritis, and other inflammatory joint and skin diseases. In one embodiment, the compositions are useful in the treatment of prostate cancer.

The present invention is also directed to methods of treatment of non-malignant tumors and other disorders involving inappropriate cell or tissue growth by administering a therapeutically effective amount of a synergistic composition of the invention. For example, it is contemplated that the invention is useful for the treatment of arteriovenous (AV) malformations, particularly in intracranial sites. The invention may also be used to treat psoriasis, a dermatologic condition that is characterized by inflammation and vascular proliferation; benign prostatic hypertrophy, a condition associated with inflammation and possibly vascular proliferation; and cutaneous fungal infections. Treatment of other hyperproliferative disorders is also contemplated. The agents may also be used topically to remove warts, birthmarks, moles, nevi, skin tags, lipomas, angiomas including hemangiomas, and other cutaneous lesions for cosmetic or other purposes.

The compositions described herein are also useful for treatment of skin. Skin is subject to deterioration through dermatological disorders, environmental abuse such as wind, air conditioning, central heating, or through the normal ageing process (chrono aging), which may be accelerated by exposure of skin to sun (photoaging). In recent years the demand for cosmetic methods for improving the appearance and condition and, in particular, for reversing, reducing or preventing the visible signs of wrinkled, aged and/or photodamaged skin has grown enormously. Consumers are increasingly seeking “anti-ageing” products that reverse, treat or delay the visible signs of chronoaging and photoaging skin such as wrinkles, lines, sagging, hyperpigmentation and age spots. Compositions and pharmaceutical formulation comprising PI3Ki and a retinoid and optionally a CYP26 inhibitor are useful for such conditions, in particular when one or both are administered via the topical route, concurrently or at different times.

Furthermore, diseases and other conditions also affect the skin that the compositions embodied herein can benefit. Diseases including acne and psoriasis are examples. Ichthyosis refers to a group of skin disorders characterized by the presence of excessive amounts of dry surface scales. This disease manifests itself within the first year of life and is clinically characterized by thickened dry, scaly skin, keratosis pilaris, tightening and cracking of the skin and many white fish-like scales. There are common and rare forms including inherited and acquired forms of ichthyosis, and an estimated total of about 1.24 million afflicted individuals in the US population. The compositions of the invention are also directed to the treatment of these various types of ichthyosis and related diseases.

In another embodiment, methods described here are provided for prevention and treatment of other cancerous and precancerous conditions, including, for example, premalignant and malignant hyperproliferative diseases such as cancers of the breast, skin, prostate, colon, bladder, cervix, uterus, stomach, lung, esophagus, blood and lymphatic system, larynx, oral cavity, metaplasias, dysplasias, neoplasias, leukoplakias and papillomas of the mucous membranes, and in the treatment of Kaposi's sarcoma. In addition, methods are provided to treat diseases of the eye, including, for example, proliferative vitreoretinopathy, retinal detachment, corneopathies such as dry eye, as well as in the treatment and prevention of various cardiovascular diseases, including, without limitation, diseases associated with lipid metabolism such as dyslipidemias, prevention of post-angioplasty restenosis and as an agent to increase the level of circulation tissue plasminogen activator. Other methods embodied herein include the prevention and treatment of conditions and diseases associated with human papilloma virus (HPV), including warts, various inflammatory diseases such as pulmonary fibrosis, ileitis, colitis and Crohn's disease, neurodegenerative diseases such as Alzheimer's disease, Parkinson's disease and stroke, improper pituitary function, including insufficient production of growth hormone, modulation of apoptosis, including both the induction of apoptosis, restoration of hair growth, including combination therapies with the present compositions and other agents such as minoxidil, diseases associated with the immune systems, including use of the present compositions as immunosuppressant and immunostimulants, modulation of organ transplant rejection and facilitation of wound healing, including modulation of chelosis. Synergistic compositions embodied herein are also useful in treating type II non-insulin dependent diabetes mellitus (NIDDM).

Non-limiting examples of PI3Ki useful in the methods and compositions herein include GDC-0941, BEZ235, BKM120, PX866, BAY 80-6946, GDC-0032, GSK 2363771, IP-145 and CAL-101, but is not limiting. GDC-0941 is a potent and selective inhibitor of phosphatidylinositol 3-kinase (PI3K) (Folkes A J et al. The identification of 2-(1H-indazol-4-yl)-6-(4-methanesulfonyl-piperazin-1-ylmethyl)-4-morpholin-4-yl-thieno[3,2-d]pyrimidine (GDC-0941) as a potent, selective, orally bioavailable inhibitor of class I PI3 kinase for the treatment of cancer. J. Med. Chem. 2008 Sep. 25; 51(18):5522-32), and compound NVP-BEZ235 has been described to potently inhibit both phosphatidylinositol 3-kinase and mammalian target of rapamycin (mTOR) (Maira S M, et al., Targeting PI3K in cancer: any good news? Identification and characterization of NVP-BEZ235, a new orally available dual phosphatidylinositol 3-kinasemammalian target of rapamycin inhibitor with potent in vivo antitumor activity. Mol. Cancer Ther. 2008 July; 7(7):1851-63). Other PI3Ki include BKM120, PX866, BAY 80-6946, GDC-0032, GSK 2363771, IP-145 and CAL-101, described in Martini et al., 2013, Targeting PI3K in cancer: any good news?, Frontiers in Oncology 2013; 3(Article 108), 1-9. Other examples of PI3Ki are described in Owonikoko T K et al., Targeting the PI3K/AKT/mTOR Pathway: Biomarkers of Success and Tribulation; ASCO Educational Book 2003; e395-e401. The invention is not limited to any particular PI3Ki and any described herein or known in the art, at the research, clinical development or approved medicine stages are embraced herein. In some instances, a PI3Km/TOR dual inhibitor compound may be used as the PI3Ki of the methods and compositions herein.

Retinoids useful for the methods and compositions of the invention include, but are not limited to, retinol, 9-cis retinoic acid, 13-cis retinoic acid, retinyl esters, all-trans retinoic acid (ATRA), and fenretinide and other atypical retinoids. Retinol ((2E,4E,6E,8E)-3,7-dimethyl-9-(2,6,6-trimethylcyclohex-1-enyl)nona-2,4,6,8-tetraen-1-ol) is one of the animal forms of vitamin A. It is a diterpenoid and an alcohol. It is convertible to other forms of vitamin A, and the retinyl ester derivative of the alcohol serves as the storage form of the vitamin in animals. Commercial production of retinol typically requires retinal synthesis through reduction of a pentadiene derivative and subsequent acidificationhydrolysis of the resulting isomer to produce retinol. Pure retinol is extremely sensitive to oxidization and is prepared and transported at low temperatures and oxygen free atmospheres. When prepared as a dietary supplement, retinol is stabilized as the ester derivatives retinyl acetate or retinyl palmitate. Synthetic retinol is marketed under the following trade names: Acon, Afaxin, Agiolan, Alphalin, Anatola, Aoral, Apexol, Apostavit, Atav, Avibon, Avita, Avitol, Axerol, Dohyfral A, Epiteliol, Nio-A-Let, Prepalin, Testavol, Vaflol, Vi-Alpha, Vitpex, Vogan, and Vogan-Neu. 9-cis-retinoic acid is also called alitretinoin. 13-cis retinoic acid is also called isotretinoin, marketed as Accutane. ATRA is also called tretinoin and is marketed under the names Aberela, Airol, Renova, Atralin, Retin-A, Retino-A, Avita, Retacnyl, Refissa, ReTrieve, or Stieva-A). Atypical retinoids include but are not limited to fenretinide which is 4-hydroxy(phenyl)retinamide, abbreviated 4-HPR; and 6-[3-(1-adamantyl)-4-hydroxyphenyl]-2-naphthalene carboxylic acid (AHPN, CD437). Any and all of the foregoing retinoids are embraced by the teachings herein.

The synergistic composition optionally comprises a CYP26 inhibitor. CYP26 inhibitors inhibit the activity of the cytochrome P450 (CYP) enzyme retinoic acid 4-hydroxylase. Numerous compounds and agents are known to inhibit CYP26 and any of these, single or in combination, are useful in the synergistic composition of the invention. CYP26 inhibitors include, by way of example, compounds described in PCTUS2011038695, published as WO2011153192, and in PCTUS201002175, published as WO2011016863, both of which are incorporated herein by reference in their entireties. These and other non-limiting examples of such CYP26 inhibitors are described below.

In one embodiment, the CYP26 inhibitor is talarozole (RAMBAZOLE), having the chemical name N-(2-benzothioazolyl)-N-[4-[2-ethyl-1-(1,2,4-triazo-1-yl)butyl]phenyl]amine. Its use as a CYP26 inhibitor is described in Stoppie et al. (2000) J Pharmacol Exp Ther. April; 293(1):304; Ocaya et al. (2007) Arterioscler Thromb Vasc Biol. July; 27(7):1542; Pavez Lorie et al. (2009) Br J Dermatol. January; 160(1):26; Armstrong et al. (2007) Br J Cancer. Jun. 4; 96(11):1675; and Giltaire et al. (2009) Br J Dermatol. March; 160(3):505.

In one embodiment, the optionally included CYP26 inhibitor useful for the methods and synergistic compositions is represented by formula (I):

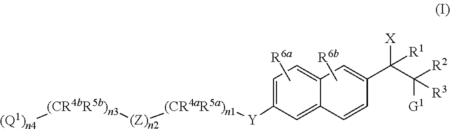

or an E or Z isomer thereof, syn or anti isomer thereof, an optically pure isomer thereof, or a pharmaceutically acceptable salt thereof, wherein:
X is an unsaturated heterocycle selected from pyrrolyl, pyrazolyl, imidazolyl, triazolyl, tetrazolyl, thiazole, or pyridinyl, any of which is optionally substituted with one or more independent R⁶⁶ substituents;
R¹ is a C₀₋₆alkyl, —OR⁷, —SR⁷, or —NR⁷R⁸;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkyl₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, mono C₁₋₆alkylaminocarbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷¹R⁸¹, —SO₂NR⁷¹R⁸¹ or —NR⁷¹R⁸¹ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR—SO₂NR⁷¹R⁸¹ or —CONR⁷¹R⁸¹ substituents;
G¹ is —OR⁷², —SR⁷², —NR⁷²R⁸²(R⁹)ₙ₅, or G¹ and R³ taken together with the carbon atom to which they are attached form a 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Y is an oxygen atom, sulfur atom, —(C═O)N(R⁷⁴)—, —CR^{4c}R^{5c} or —NR⁷⁴;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with R⁶⁸;

Q¹ is C₀₋₆alkyl, —OR⁷⁵, —NR⁷⁵R⁸⁵(R⁹⁵)ₙ₆, —CO₂R⁷⁵, —CONR⁷⁵R⁸⁵, —(C═S)OR⁷⁵, —(C═O)SR⁷⁵, —NO², —CN, halo, —S(O)ₙ₆R⁷⁵, —SO₂NR⁷⁵R⁸⁵, —NR⁷⁵(C═NR⁷⁷⁵)NR⁷⁷⁷⁵R⁸⁵, —NR⁷⁵(C═NR⁷⁷⁵)OR⁷⁷⁷⁵, —NR⁷⁵(C═NR⁷⁷⁵)SR⁷⁷⁷⁵, —O(C═O)OR⁷⁵, —O(C═O)NR⁷⁵R⁸⁵, —O(C═O)SR⁷⁵, —S(C═O)OR⁷⁵, —S(C═O)NR⁷⁵R⁸⁵, —S(C═O)SR⁷⁵, —NR⁷⁵(C═O)NR⁷⁷⁵R⁸⁵, or —NR⁷⁵(C═S)NR⁷⁷⁵R⁸⁵; in the case of —NR⁷⁵R⁸⁵ (R⁹⁵)ₙ₆, R⁷⁵ and R⁸⁵ taken together with the nitrogen atom to which they are attached form a 3-10 membered saturated ring, unsaturated ring, heterocyclic saturated ring, or heterocyclic unsaturated ring, any of which is optionally substituted with one or more independent halo, cyano, hydroxy, nitro, C₁₋₁₀alkoxy, —SO₂N^{R6}R⁸⁶ or —NR⁷⁶R⁸⁶ substituents;

R^{4a}, R^{4b}, R^{4c}, R^{5a}, R^{5b} and R^{5c} are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or R^{4a} with R^{5a}, or R^{4b} with R^{5b}, or R^{4c} with R^{5c}, taken together with the respective carbon atom to which they are attached, form a carbonyl or 3-10 membered saturated or unsaturated ring, wherein said ring is optionally substituted with R⁶⁹; or R^{4a} with R^{5a}, or R^{4b} with R^{5b}, or R^{4c} with R^{5c}, taken together with the respective carbon atom to which they are attached, form a 3-10 membered saturated or unsaturated heterocyclic ring, wherein said ring is optionally substituted with R⁶⁹;
R^{6a}, R^{6b}, R⁶⁶, R⁶⁷, R⁶⁸, and R⁶⁹ are each independently halo, —OR—SH, —NR⁷⁷R⁸⁸(R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, C₁₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)amino C₁₋₆alkyl, di(C₁₋₆alkyl)aminoC₁₋₆alkyl, mono(aryl)aminoC₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R85, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl) or —N(C₀₋₄ alkyl) (C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₁₀alkyl), —SO₂N(C₀₋₄alkyl)(C₀₋₄alkyl) or —N(C₀₋₄ alkyl)(C₀₋₄ alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₄ alkyl), —SO₂N(C₀₋₄ alkyl)(C₀₋₄ alkyl) or —N(C₀₋₄ alkyl) (C₀₋₄alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₄alkyl), —SO₂N(C₀₋₄alkyl)(C₀₋₄alkyl) or —N(C₀₋₄ alkyl) (C₀₋₄ alkyl) substituents; and
n1, n2, n3, n4, n5, n6, and n7 are each independently equal to 0, 1 or 2.

In another embodiment, the compound is represented by Formula I, or a pharmaceutically acceptable salt thereof, wherein X is an optionally substituted imidazolyl or optionally substituted triazolyl, and the other variables are as described above.

In an embodiment of this aspect, a compound is represented by Formula I, or a pharmaceutically acceptable salt thereof, wherein X is a substituted imidazolyl or substituted triazolyl; R¹ is hydrogen; and the other variables are as described above.

In another embodiment, the compound is represented by Formula I, or a pharmaceutically acceptable salt thereof, wherein Y is oxygen, and the other variables are as described above.

In one embodiment, the CYP26 inhibitor is represented by Formula IA:

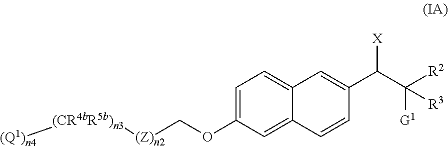

or an E or Z isomer thereof, syn or anti isomer thereof, an optically pure isomer thereof, or a pharmaceutically acceptable salt thereof, wherein:
X is an unsaturated heterocycle selected from pyrrolyl, pyrazolyl, imidazolyl, triazolyl, tetrazolyl, thiazole, or pyridinyl, any of which is optionally substituted with one or more independent R⁶⁶ substituents;
R² and R³ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthio C₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, mono C₁₋₆alkylaminocarbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷¹R⁸¹, —SO₂NR⁷¹R⁸¹ or —NR⁷¹R⁸¹ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR—SO₂NR⁷¹R⁸¹ or —CONR⁷¹R⁸¹ substituents;
G¹ is —OR⁷², —SR⁷², —NR⁷²R⁸²(R⁹)ₙ₅, or G¹ and R³ taken together with the carbon atom to which they are attached form a 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with R⁶⁸;
Q¹ is C₀₋₆alkyl, —OR⁷⁵, —NR⁷⁵R⁸⁵(R⁹⁵)ₙ₆, —CO₂R⁷⁵, —CONR⁷⁵R⁸⁵, —(C═S)OR⁷⁵, —(C═O)SR⁷⁵, —NO², —CN, halo, —S(O)ₙ₆R⁷⁵, —SO₂NR⁷⁵R⁸⁵, —NR⁷⁵(C═NR⁷⁷⁵)NR⁷⁷⁷⁵R⁸⁵, —NR⁷⁵(C═NR⁷⁷⁵)OR⁷⁷⁷⁵, —NR⁷⁵(C═NR⁷⁷⁵)SR⁷⁷⁷⁵, —O(C═O)OR⁷⁵, —O(C═O)NR⁷⁵R⁸⁵, —O(C═O)SR⁷⁵, —S(C═O)OR⁷⁵, —S(C═O)NR⁷⁵R⁸⁵, —S(C═O)SR⁷⁵, —NR⁷⁵(C═O)NR⁷⁷⁵R⁸⁵, or —NR⁷⁵(C═S)NR⁷⁷⁵R⁸⁵; in the case of —NR⁷⁵R⁸⁵ (R⁹⁵)ₙ₆, R⁷⁵ and R⁸⁵ taken together with the nitrogen atom to which they are attached form a 3-10 membered saturated ring, unsaturated ring, heterocyclic saturated ring, or heterocyclic unsaturated ring, any of which is optionally substituted with one or more independent halo, cyano, hydroxy, nitro, C1-10alkoxy, —SO₂N^{R6}R⁸⁶ or —NR⁷⁶R⁸⁶ substituents;
R^{4b} and R^{5b} are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkyl C₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or R^{4b} with R^{5b}, taken together with the respective carbon atom to which they are attached, form a carbonyl or 3-10 membered saturated or unsaturated ring, wherein said ring is optionally substituted with R⁶⁹; or R^{4b} with R^{5b}, taken together with the respective carbon atom to which they are attached, form a 3-10 membered saturated or unsaturated heterocyclic ring, wherein said ring is optionally substituted with R⁶⁹;
R⁶⁶, R⁶⁷, R⁶⁸, and R⁶⁹ are each independently —OR⁷⁸, —NR⁷⁷R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)aminoC₁₋₆alkyl, di(C₁₋₆alkyl)aminoC₁₋₆alkyl, mono(aryl)amino C₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R85, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynmyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄ alkyl)(C₀₋₄alkyl) or —N(C₀₋₄alkyl) (C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄ alkyl)(C₀₋₄alkyl) or —N(C₀₋₄ alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₄alkyl), —SO₂N(C₀₋₄ alkyl)(C₀₋₄alkyl) or —N(C₀₋₄alkyl) (C₀₋₄alkyl) substituents; or mono(C₁₋₆alkyl)aminoC₁₋₆alkyl, di(C₁₋₆alkyl)aminoC₁₋₆ alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CON(C₀₋₄ alkyl)(C₀₋₄alkyl), —SO₂N(C₀₋₄ alkyl)(C₀₋₄alkyl) or —N(C₀₋₄alkyl) (C₀₋₄alkyl) substituents;
and
n2, n3, n4, n5, n6, and n7 are each independently equal to 0, 1 or 2.

In another embodiment the CYP26 inhibitor is represented by Formula 1-B:

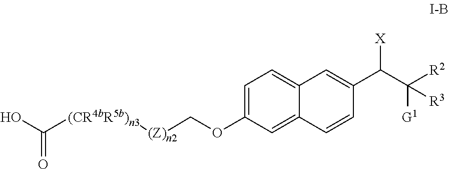

or an E or Z isomer thereof, syn or anti isomer thereof, an optically pure isomer thereof, or a pharmaceutically acceptable salt thereof, wherein:
X is substituted imidazolyl;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, mono C₁₋₆alkylaminocarbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR—SO₂NR⁷¹R⁸¹ or —CONR⁷¹R⁸¹ substituents;
G¹ is —OR⁷², —SR⁷², —NR⁷²R⁸²(R⁹)ₙ₅, or G¹ and R³ taken together with the carbon atom to which they are attached form a 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with R⁶⁸;
R^{4b} and R^{5b} are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkyl C₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or R^{4b} with R^{5b}, taken together with the respective carbon atom to which they are attached, form a carbonyl or 3-10 membered saturated or unsaturated ring, wherein said ring is optionally substituted with R⁶⁹;
R⁶⁷, R⁶⁸ and R⁶⁹ are each independently —OR⁷⁸, —NR⁷⁷R⁸⁸(R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, C₁₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)aminoC₁₋₆alkyl, di(C₁₋₆alkyl)aminoC₁₋₆alkyl, mono(aryl)amino C₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R⁸⁵, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀ alkoxycarbonylC₁₋₁₀alkyl, mono C₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄ alkyl)(C₀₋₄alkyl) or —N(C₀₋₄ alkyl) (C₀₋₄ alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄ alkyl)(C₀₋₄alkyl) or —N(C₀₋₄ alkyl)(C₀₋₄ alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₄ alkyl), —SO₂N(C₀₋₄ alkyl)(C₀₋₄alkyl) or —N(C₀₋₄ alkyl) (C₀₋₄ alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CON(C₀₋₄ alkyl)(C₀₋₄ alkyl), —SO₂N(C₀₋₄ alkyl)(C₀₋₄alkyl) or —N(C₀₋₄ alkyl) (C₀₋₄ alkyl) substituents; and
n2, n3, n4, n5, n6, and n7 are each independently equal to 0, 1 or 2.

The compounds of the present invention include compounds represented by Formula I above, or a pharmaceutically acceptable salt thereof, and

1) wherein X is hetaryl, imidazolyl, or triazolyl, any of which is optionally substituted with one or more independent R⁶⁶ substituents; or
2) wherein X is imidazolyl or triazolyl; or
3) wherein X is hetaryl, imidazolyl, or triazolyl, any of which is optionally substituted with one or more independent R⁶⁶ substituents, and Q¹ is —CO₂H or —CO₂R⁷⁵; or
4) wherein Y is oxygen; or
5) wherein Y is oxygen and X is hetaryl, imidazolyl, or triazolyl, any of which is optionally substituted with one or more independent R⁶⁶ substituents; or
6) wherein Y is oxygen and X is imidazolyl or triazolyl; or
7) wherein Y is oxygen and X is imidazolyl or triazolyl and Q¹ is —CO₂H or —CO₂R⁷⁵; or
8) wherein Y is oxygen and R^{4a} and R^{5a} are each hydrogen; or
9) wherein X is hetaryl, imidazolyl, or triazolyl, any of which is optionally substituted with one or more independent R⁶⁶ substituents; R¹, R² and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁴R⁸⁵; R^{4a}, R^{4b}, R^{1a}, and R^{1b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a} or R^{4b} with R^{5b} taken together with the respective carbon atom to which they are attached form a 3-10 membered saturated or unsaturated heterocyclic ring, wherein said ring is optionally substituted with R⁶⁹; and are each independently halo, —OR⁷⁸, CONR⁷⁸R⁸⁸, —NO₂, —CN, —S(O)ₙ₇R⁷⁸, —SO₂NR⁷⁸R⁸⁸, or C₀₋₁₀alkyl; or
10) wherein X is imidazolyl or triazolyl; R¹ is hydrogen, R² and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is —CO₂R⁷⁵ or —CONR⁷⁵R⁸⁵; R^{4a}, R^{4b}, R^{5a} and R^{5b} are each independently a C₀₋₁₀alkyl, any of which is optionally substituted with one or more independent halo, cyano, nitro, —OR, —SO₂ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each hydrogen; or
11) wherein X is hetaryl, imidazolyl, or triazolyl, any of which is optionally substituted with one or more independent R⁶⁶ substituents; R¹, R² and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R or —CONR⁷⁵R⁸⁵; R^{4a} and R^{5a} are each hydrogen; R^{4b} and R^{5b} are each independently a C₀₋₁₀alkyl, any of which is optionally substituted with R⁶⁹; or R^{4b} with R^{5b} taken together with the respective carbon atom to which they are attached form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; or
12) wherein X is hetaryl, imidazolyl, or triazolyl, any of which is optionally substituted with one or more independent R⁶⁶ substituents; R¹, R² and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; or
13) wherein X is hetaryl, imidazolyl, or triazolyl, any of which is optionally substituted with one or more independent R⁶⁶ substituents; R¹, R² and R³ are each independently C₁₀₋₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, or C₀₋₁₀alkyl; and R^{4b} with R^{5b} taken together with the respective carbon atom to which they are attached form a cyclopropyl, cyclobutyl, cyclopentyl, or cyclohexyl ring; or
14) wherein X is hetaryl, imidazolyl, or triazolyl, any of which is optionally substituted with one or more independent R⁶⁶ substituents; R¹, R² and R³ are each independently C₁₀₋₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and R^{4b} and R^{5b} are both ethyl or are both methyl or are independently ethyl or methyl; or
15) wherein X is hetaryl, imidazolyl, or triazolyl, any of which is optionally substituted with one or more independent R⁶⁶ substituents; R¹, R² and R³ are each independently C₁₀₋₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO², —CN, —S(O)ₙ7R⁷⁸, —SO₂NR⁷⁸R⁸⁸, or C₀₋₁₀alkyl; and Q¹ is —CO₂R; or
16) wherein X is hetaryl, imidazolyl, or triazolyl, any of which is optionally substituted with one or more independent R⁶⁶ substituents; R¹, R² and R³ are each independently C₁₀₋₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO², —CN, —S(O)ₙ7R⁷⁸, —SO₂NR⁷⁸R⁸⁸, or C₀₋₁₀alkyl; and Q¹ is —CO₂H; or
17) wherein X is hetaryl, imidazolyl, or triazolyl, any of which is optionally substituted with one or more independent R⁶⁶ substituents; R¹, R² and R³ are each independently C₀₋₁₀alkyl; Y is oxygen; Q¹ is C₀₋₆alkyl, CO₂R⁷⁵, or —CoNR⁷⁵R⁸⁵; R⁴, R^{4b}, R^{5a}, and R^{5b} are each independently a C₀₋₁₀alkyl, any of which is optionally substituted with one or more independent halo, cyano, nitro, —OR⁷⁷—SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸ (R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, or C₀₋₁₀alkyl; and G¹ is di(C₁₋₆alkyl)amino; or
18) wherein X is hetaryl, imidazolyl, or triazolyl, any of which is optionally substituted with one or more independent R⁶⁶ substituents; R¹, R² and R³ are each independently C₀₋₁₀alkyl; Y is oxygen; Q¹ is C₀₋₆alkyl, CO₂R⁷⁵, or —CoNR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷—SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸ (R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and G¹ is di(C₁₋₆alkyl)amino, ethylmethylamino, diethylamino, or isopropylmethylamino; or
19) wherein X is hetaryl, imidazolyl, or triazolyl, any of which is optionally substituted with one or more independent R⁶⁶ substituents; R¹ is C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, or C₀₋₁₀alkyl; and R² and R³ are each independently hydrogen, methyl, or ethyl; or
20) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, or C₀₋₁₀alkyl; R² is hydrogen; and G¹ and R³ taken together with the carbon atom to which they are attached form

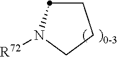

wherein, is the carbon to which they are attached;
or G¹ and R³ taken together with the carbon atom to which they are attached form

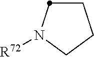

wherein is the carbon to which they are attached, any of which is optionally substituted by 1-10 independent R⁶⁷ substituents; or

21) wherein X is imidazole; or
22) wherein X is hetaryl, imidazolyl, or triazolyl, any of which is optionally substituted with one or more independent R⁶⁶ substituents; R¹ is C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R are each independently halo, —OR⁷⁸, —NR⁷⁸R⁸⁸(R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, or C₀₋₁₀alkyl; and R² is hydrogen and R³ is methyl; or 23) wherein X is hetaryl, imidazolyl, or triazolyl, any of which is optionally substituted with one or more independent R⁶⁶ substituents; R1 is C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, or C₀₋₁₀alkyl; and R² is hydrogen and R³ is ethyl; or 24) wherein X is hetaryl, imidazolyl, or triazolyl, any of which is optionally substituted with one or more independent R⁶⁶ substituents; R¹ is C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, or C₀₋₁₀alkyl; and R² and R³ are both methyl; or
25) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and R² is hydrogen; and G¹ and R³ taken together with the carbon atom to which they are attached form

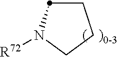

wherein is the carbon to which they are attached, or G¹ and R³ taken together with the carbon atom to which they are attached form

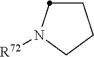

wherein is the carbon to which they are attached, any of which is optionally substituted by 1-10 independent R⁶⁷ substituents; and n2, n3, and n4 are each 1 and Z is aryl; or

26) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}; R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and R² is hydrogen; and G¹ and R³ taken together with the carbon atom to which they are attached form

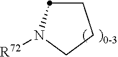

wherein is the carbon to which they are attached, or G¹ and R³ taken together with the carbon atom to which they are attached form

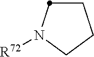

wherein is the carbon to which they are attached, any of which is optionally substituted by 1-10 independent R⁶⁷ substituents; n2 is 1; n3 and n4 are each 0; and Z is aryl; or
27) wherein Z is aryl or aryloxy or oxyaryl; or
28) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, or C₀₋₁₀alkyl; and R² is hydrogen; and G¹ and R³ taken together with the carbon atom to which they are attached form

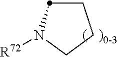

wherein is the carbon to which they are attached, or G¹ and R³ taken together with the carbon atom to which they are attached form

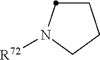

wherein is the carbon to which they are attached, any of which is optionally substituted by 1-10 independent R⁶⁷ substituents; and n2, n3, and n4 are each 1 and Z is aryl; and n3 is 0; or
29) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and R² is hydrogen; and G¹ and R³ taken together with the carbon atom to which they are attached form

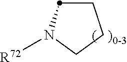

wherein is the carbon to which they are attached, or G¹ and R³ taken together with the carbon atom to which they are attached form

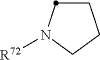

wherein is the carbon to which they are attached, any of which is optionally substituted by 1-10 independent R⁶⁷ substituents; and n2, n3, and n4 are each 1 and Z is aryl; and n3 is 0; or 30) wherein X is imidazolyl or triazolyl; R¹ is hydrogen; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is —CO₂R⁷⁵ or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each hydrogen; R² is hydrogen; and R³ is methyl; or
31) wherein X is imidazolyl or triazolyl; R¹ is hydrogen; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is —CO₂R⁷⁵ or —CONR⁷⁵R⁸⁵; R^{4a}; R^{4b}; R^{5a}, and R^{5b} are each independently a C₁₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each hydrogen; R² is hydrogen; and R³ is ethyl; or
32) wherein X is imidazolyl or triazolyl; R¹ is hydrogen; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is —CO₂R⁷⁵ or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each hydrogen; and R² and R³ are methyl; or
33) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)n7, —CO₂R⁷⁸, —CONR⁷⁸R⁸⁸, —NO², —CN, —S(O)ₙ₇R⁷⁸, —SO₂NR⁷⁸R⁸⁸, or C₀₋₁₀alkyl; R² is hydrogen; and G¹ and R³ taken together with the carbon atom to which they are attached form

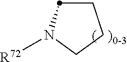

wherein is the carbon to which they are attached, or G¹ and R³ taken together with the carbon atom to which they are attached form

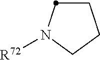

wherein is the carbon to which they are attached, any of which is optionally substituted by 1-10 independent R substituents; n1 and n2 are each 1; and Z is aryl; or
34) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)n7, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; R² is hydrogen; and G¹ and R³ taken together with the carbon atom to which they are attached form

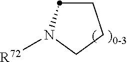

wherein is the carbon to which they are attached, or G¹ and R³ taken together with the carbon atom to which they are attached form

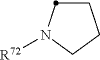

wherein is the carbon to which they are attached, any of which is optionally substituted by ₁₋₁₀ independent R⁶⁷ substituents; n1 and n2 are each 1; n3 and n4 are each 0; and Z is aryl; or
35) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)n7, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; R² is hydrogen; and G¹ and R³ taken together with the carbon atom to which they are attached form

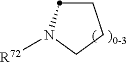

wherein is the carbon to which they are attached, or G¹ and R³ taken together with the carbon atom to which they are attached form

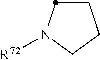

wherein is the carbon to which they are attached, any of which is optionally substituted by ₁₋₁₀ independent R⁶⁷ substituents; n1 and n2 are each 1; Z is aryl; and Q¹ is —CO₂R⁷⁵; or
36) wherein X is hetaryl, imidazolyl, or triazolyl, any of which is optionally substituted with one or more independent R⁶⁶ substituents; R¹ and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R⁷⁵, or —CONR⁷⁵R⁸⁵; R^{4a}; R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)n7, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; R² is hydrogen; and G¹ and R³ taken together with the carbon atom to which they are attached form

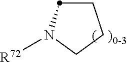

wherein is the carbon to which they are attached, or G¹ and R³ taken together with the carbon atom to which they are attached form

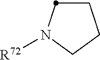

wherein is the carbon to which they are attached, any of which is optionally substituted by ₁₋₁₀ independent R⁶⁷ substituents; n1 and n2 are each 1; Z is aryl; and Q¹ is —CO₂H; and wherein, in each case, the other variables are as defined above for Formula I.

The CYP26 inhibitor compounds useful in the compositions of the present invention include, by way of non-limiting examples: methyl 3-((6-(2-(dimethylamino)-1-(1H-imidazol-1-yl)propyl)naphthalen-2-yl)oxy)-2,2-dimethylpropanoate; methyl 2-(((6-(2-(dimethylamino)-1-(1H-imidazol-1-yl)propyl)naphthalen-2-yl)oxy)methyl)-2-ethylbutanoate; ethyl 1-(((6-(2-(dimethylamino)-1-(1H-imidazol-1-yl)propyl)naphthalen-2-yl)oxy)methyl)cyclopropanecarboxylate; ethyl 1-(((6-(2-(dimethylamino)-1-(1H-imidazol-1-yl)propyl)naphthalen-2-yl)oxy)methyl)cyclobutanecarboxylate; methyl 1-(((6-(2-(dimethylamino)-1-(1H-imidazol-1-yl)propyl)naphthalen-2-yl)oxy)methyl)cyclopentanecarboxylate; methyl 1-(((6-(2-(dimethylamino)-1-(1H-imidazol-1-yl)propyl)naphthalen-2-yl)oxy)methyl)cyclohexanecarboxylate; methyl 1-(((6-(1-(1H-imidazol-1-yl)-2-(isopropyl(methyl)amino)propyl)naphthalen-2-yl)oxy)methyl)cyclopentanecarboxylate; methyl 3-((6-(2-(diethylamino)-1-(1H-imidazol-1-yl)propyl)naphthalen-2-yl)oxy)-2,2-dimethylpropanoate; methyl 3-((6-(1-(1H-imidazol-1-yl)-2-(isopropyl(methyl)amino)propyl)naphthalen-2-yl)oxy)-2,2-dimethylpropanoate; methyl 3-((6-(2-(ethyl(methyl)amino)-1-(1H-imidazol-1-yl)propyl)naphthalen-2-yl)oxy)-2,2-dimethylpropanoate; methyl 3-((6-(2-(dimethylamino)-1-(1H-imidazol-1-yl)butyl)naphthalen-2-yl)oxy)-2,2-dimethylpropanoate; methyl 4-(((6-(2-(dimethylamino)-1-(1H-imidazol-1-yl)propyl)naphthalen-2-yl)oxy)methyl)benzoate; methyl 3-(((6-(2-(dimethylamino)-1-(1H-imidazol-1-yl)propyl)naphthalen-2-yl)oxy)methyl)benzoate; 3-[6-(2-dimethyl amino-1-imidazol-1-yl-propyl)-naphthalen-2-yloxy]-2,2-dimethyl-propionic acid; 2-[6-(2-dimethylamino-1-imidazol-1-yl-propyl)-naphthalen-2-yloxymethyl]-2-ethyl-butyric acid; 1-[6-(2-dimethylamino-1-imidazol-1-yl-propyl)-naphthalen-2-yloxymethyl]-cyclopropanecarboxylic acid; [6-(2-dimethylamino-1-imidazol-1-yl-propyl)-naphthalen-2-yloxymethyl]-cyclobutanecarboxylic acid; [6-(2-dimethyl amino-1-imidazol-1-yl-propyl)-naphthalen-2-yloxymethyl]-cyclopentanecarboxylic acid; 1-[6-(2-dimethylamino-1-imidazol-1-yl-propyl)-naphthalen-2-yloxymethyl]-cyclohexanecarboxylic acid; 1-{6-[1-imidazol-1-yl-2-(isopropylmethylamino)-propyl]-naphthalen-2-yloxymethyl}-cyclopentanecarboxylic acid; 3-[6-(2-diethylamino-1-imidazol-1-yl-propyl)-naphthalen-2-yloxy]-2,2-dimethyl-propionic acid; {6-[1-imidazol-1-yl-2-(isopropylmethylamino)-propyl]-naphthalen-2-yloxy}-2,2-dimethyl-propionic acid; 3-6-[2-(ethyl-methyl-amino)-1-imidazol-1-yl-propyl]-naphthalen-2-yloxy}-2,2-dimethyl-propionic acid; 3-[6-(2-dimethylamino-1-imidazol-1-yl-propyl)-naphthalen-2-yloxy]-2,2-dimethyl-propionamide; 3-[6-(2-dimethylamino-1-imidazol-1-yl-propyl)-naphthalen-2-yloxy]-2,2, n-trimethyl-propionamide; [6-(2-dimethylamino-1-imidazol-1-yl-propyl)-naphthalen-2-yloxy]-2,2, N,N-tetramethyl-propionamide; 3-[6-(2-dimethylamino-1-imidazol-1-yl-butyl)-naphthalen-2-yloxy]-2,2-dimethyl-propionic acid; 4-[6-(2-dimethylamino-1-imidazol-1-yl-propyl)-naphthalen-2-yloxymethyl]-benzoic acid; 3-[6-(2-dimethylamino-1-imidazol-1-yl-propyl)-naphthalen-2-yloxymethyl]-benzoic acid; 4-[6-(2-dimethylamino-1-imidazol-1-yl-propyl)-naphthalen-2-yloxymethyl]-benzamide; 4-[6-(2-dimethylamino-1-imidazol-1-yl-propyl)-naphthalen-2-yloxymethyl]-n-methyl-benzamide; 4-[6-(2-dimethyl amino-1-imidazol-1-yl-propyl)-naphthalen-2-yloxymethyl]-N, N-dimethyl-benzamide; and 1-[(6-benzyloxy-naphthalen-2-yl)-(1-methyl-pyrrolidin-2-yl)-methyl]-1H-imidazole.

In another embodiment, the CYP26 inhibitor is represented by formula (II)

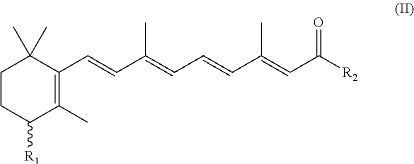

and E or Z isomers thereof, syn or anti isomers thereof, optically pure isomers thereof, or pharmaceutically acceptable salt thereof, and a pharmaceutically acceptable carrier therefor, wherein:

R₁ is an optionally substituted azole, sulfur, oxygen, nitrogen, pyridyl, acetylinic, cyclopropyl-amine, ester, oxime, cyano, amino, azido, cyclopropylamino, oxirane, aziridine, thiirane, thiol, alkylthiol, —OR⁴ wherein R₄ is hydrogen or an alkyl group, cyclopropylether, an oxygen containing group that forms, together with the 4-position carbon, an oxirane group; —NR⁵R⁶, where R⁵ and R⁶ are independently selected from the group consisting of hydrogen and alkyl groups, or R⁵ and R⁶ may together form a ring; and

R₂ is selected from the group consisting of hydroxyl, aminophenol, —OR₃ and an azole group, wherein R₃ is selected from the group consisting of alkyl, aryl and heterocyclic groups;
In certain embodiments, R₁ is selected from the group consisting of sulfur containing groups, oxygen containing groups, nitrogen containing groups, acetylinic, ester groups, oxime and aziridine; and R₂ is selected from the group consisting of hydroxyl, aminophenol, —OR³ and azole groups, wherein R₃ is selected from the group consisting of alkyl, aryl and heterocyclic groups.

In other embodiments, R₁ is optionally substituted azole, sulfur, oxygen, nitrogen, pyridyl, acetylinic, cyclopropyl-amine, esters, oxime, cyano, oxirane or aziridine; and R₂ is hydroxyl, an aminophenol, an ester, or an azole.

R₁ may be a sulfur containing group. Examples of such sulfur containing groups include thiirane, thiol and alkylthiol derivatives. Examples of such alkylthiol derivatives include C₁ to C₁₀alkyl thiols.

R₁ may be an oxygen containing group. Examples of oxygen containing groups include —OR₄, where R₄ is hydrogen or an alkyl group (preferably a 1-10 carbon alkyl, more preferably methyl or ethyl), cyclopropylether or an oxygen containing group that forms, together with the 4-position carbon, an oxirane group.

R₁ may be a nitrogen containing group. Examples of such nitrogen containing groups include the formula —NR⁵R⁶, where R⁵ and R⁶ are independently selected from the group consisting of hydrogen and alkyl groups (preferably a 1-10 carbon alkyl, more preferably methyl or ethyl), or R⁵ and R⁶ may together form a ring. Preferably the ring formed by R⁵ and R⁶ is an imidazolyl ring or a triazole ring.

Preferable azole substituent groups include imidazoles and triazoles. More preferably, the azole substituent groups include 1H imidazole-1-yl, 1H 1,2,4-triazol-1-yl and 4H-1,2,4-triazol-4-yl.

R₁ may be a cyano, amino, azido, cyclopropylamino, or R₁ is a nitrogen containing group that forms, together with the 4-position carbon, an aziridine group or an oxime group.

R₁ may also be a pyridyl group or an allylic azole group, preferably methyleneazolyl.

The definitions for R₁ of an ester includes substituent groups that contain an ester moiety, including substituent groups attached via an ester moiety.

R₂ may be preferably selected from the group consisting of hydroxyl, aminophenol, —OR³ and azole groups, wherein R³ is selected from the group consisting of alkyl, aryl and heterocyclic groups, more preferably, hydroxyl or —OCH₃ (methoxy).

Said alkyl substituents for the above identified substituent groups include substituted and unsubstituted alkyl groups, branched and straight chain and cyclo alkyl groups, such as cyclopropyl.

The term “aryl” includes a phenyl or naphthyl ring.

Non-limiting examples of CYP26 inhibitors of formula (II) useful in the compositions and methods for the purposes described herein include: (±)-4-(1H-imidazol-1-yl)-methyl retinoate, (±)-4-(1H-imidazole-1-yl)retinoic acid, (±)-4-(1H-1,2,4-triazol-1-yl) methyl retinoate, (±)-4-(4H-1,2,4-triazole-4-yl) methyl retinoate, (±)-4-(1H-1,2,4-triazol-1-yl) retinoic acid, and (±)-4-(4H-1,2,4-triazol-4-yl) retinoic acid.

Some of the foregoing compounds in formulas (I), (I-A), (I-B) and (II) can comprise one or more asymmetric centers, and thus can exist in various isomeric forms, e.g., stereoisomers and/or diastereomers. Thus, compositions herein comprising such compounds may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

In another embodiment, the CYP26 inhibitor of the synergistic composition is represented by formula (III)

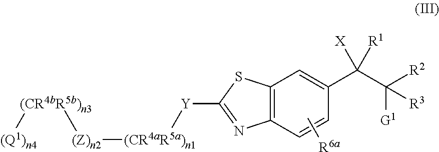

or an E or Z isomer thereof, syn or anti isomer thereof, an optically pure isomer thereof, or pharmaceutically acceptable salt thereof, wherein:
X is an unsaturated heterocycle selected from pyrrolyl, pyrazolyl, imidazolyl, triazolyl, benzimidazolyl, benzotriazolyl, tetrazolyl, thiazole, 3-pyridinyl or 4-pyridinyl, any of which is optionally substituted with one or more independent R⁶⁶ substituents;
R¹ is hydrogen, C₀₋₆alkyl, —OR⁷, —SR⁷, or —NR⁷R⁸;
R₂ and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, mono C₁₋₆alkylaminocarbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR⁷⁷—SO₂NR⁷¹R⁸¹ or —CONR⁷¹R⁸¹ substituents;
G¹ is hydrogen, C₀₋₁₀alkyl, C₂₋₁₀alkenyl, C₂₋₁₀alkynyl, —OR⁷², —SR⁷², —NR⁷²R⁸²(R⁹)ₙ₅, or G¹ and R³ taken together with the carbon atom to which they are attached forma 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Y is a bond, hydrogen, halogen, an oxygen atom, sulfur atom, —CN, R⁷⁴, —C(═NR⁷⁴)NR⁷⁵R⁷⁶, —C(═NR⁷⁴)OR⁷⁵—, —COR⁷⁴, —R⁷⁴COR⁷⁵, —CR⁷⁴R⁷⁵COR⁷⁶, —COOR⁷⁴, —R⁷⁴COOR⁷⁵, —CR⁷⁴R⁷⁵COOR⁷⁶, —CONR⁷⁴R⁷⁵, —CR⁷⁴R⁷⁵CONR⁷⁴R⁷⁵, —NR⁷⁴COOR⁷⁵, —NR⁷⁴COR⁷⁵, NR⁷⁴CONR⁷⁵R⁷⁶, —SO₂CR⁷⁴R⁷⁵, —SO₂NR⁷⁴R⁷⁵, —NR⁷⁴SO₂NR⁷⁵R⁷⁶, —NR^{6b}CO—, —NR^{6b}R⁷⁵, aryl or heteroaryl, any of which is optionally substituted with one or more independent halo, cyano, hydroxy, nitro, R⁶⁸, —SO₂N^{R6}R⁸⁶ or —NR⁷⁶R⁸⁶ substituents;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with one or more independent halo, cyano, hydroxy, nitro, R⁶⁸, C₁₋₁₀alkoxy, —COOR⁷⁴, —(C═O)N(R⁷⁴)—, —NR⁷⁴CONR⁷⁴R⁷⁵, —NR⁷⁴COOR⁷⁵, —SO₂N^{R6}R⁸⁶, —NR⁷⁴SO₂NR⁷⁴R⁷⁵, or —NR⁷⁶R⁸⁶ substituents;
Q¹ is C₀₋₆alkyl, cycloC₃₋₈alkyl, bridged bicycloalkyl, —OR⁷⁵, —COR⁷⁴, —NR⁷⁵R⁸⁵(R⁹⁵)ₙ₆, —CO₂R⁷⁵, —CONR⁷⁵R⁸⁵, —(C═S)OR⁷⁵, —(C═O)SR⁷⁵, —NO₂, —CN, halo, —S(O)ₙ₆R⁷⁵, —SO₂NR⁷⁵R⁸⁵, —NR⁷⁵(C═NR⁷⁷⁵)NR⁷⁷⁷⁵R⁸⁵, —NR⁷⁵(C═NR⁷⁷⁵)OR⁷⁷⁷⁵, —NR⁷⁵(C═NR⁷⁷⁵)SR⁷⁷⁷⁵, —O(C═O)OR⁷⁵, —O(C═O)NR⁷⁵R⁸⁵, —O(C═O)SR⁷⁵, —S(C═O)OR⁷⁵, —S(C═O)NR⁷⁵R⁸⁵, —S(C═O)SR⁷⁵, —NR⁷⁵(C═O)NR⁷⁷⁵R⁸⁵, or —NR⁷⁵(C═S)NR⁷⁷⁵R⁸⁵; in the case of—NR⁷⁵R⁸⁵(R⁹⁵)ₙ₆, R⁷⁵ and R⁸⁵ taken together with the nitrogen atom to which they are attached form a 3-10 membered heterocyclic saturated ring, or heterocyclic unsaturated ring, any of which is optionally substituted with one or more independent halo, cyano, hydroxy, nitro, C₁₋₁₀alkoxy, —SO₂N^{R6}R⁸⁶ or —NR⁷⁶R⁸⁶ substituents;
R^{4a}, R^{4b}, R^{4c}, R^{5a}, R^{5b} and R^{5c} are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono(C₁₋₆alkyl)amino C₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or R^{4a} with R^{5a}, or R^{4b} with R^{5b}, or R^{4c} with R^{5C}, taken together with the respective carbon atom to which they are attached, form a carbonyl or 3-10 membered saturated or unsaturated monocyclic or polycyclic ring, wherein said ring is optionally substituted with R⁶⁹; or R^{4a} with R^{5a}, or R^{4b} with R^{5b}, or R^{4c} with R^{5c}, taken together with the respective carbon atom to which they are attached, form a 3-10 membered saturated or unsaturated monoheterocyclic or polyheterocyclic ring, wherein said ring is optionally substituted with R⁶⁹;
R^{6a}, R^{6b}, R⁶⁶, R⁶⁷, R⁶⁸, and R⁶⁹ are each independently hydrogen, halo, —OR⁷⁷—SH, —NR⁷⁷R⁸⁸(R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, C₁₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthio C₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)amino C₁₋₆alkyl, di(C₁₋₆alkyl)aminoC₁₋₆alkyl, mono(aryl)amino C₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered mono or polycyclic saturated ring, mono or polycyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R⁸⁵, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynmyl, C₁₋₁₀alkylthioC₁₋₁₀alkyl, C₁₋₁₀alkylthio C₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl) or —N(C₀₋₄alkyl) (C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl) (C₀₋₄alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₄alkyl), —SO₂N(C₀₋₄ alkyl)(C₀₋₄alkyl) or —N(C₀₋₄alkyl) (C₀₋₄alkyl) substituents; and
n1, n2, n3, n4, n5, n6, and n7 are each independently equal to 0, 1 or 2.
   In another embodiment, a compound is represented by Formula (III), or a pharmaceutically acceptable salt thereof, wherein X is an optionally substituted imidazolyl, optionally substituted triazolyl, optionally substituted 3-pyridinyl, optionally substituted 4-pyridinyl and the other variables are as described above.

In another embodiment, a compound is represented by Formula (III), or a pharmaceutically acceptable salt thereof, wherein X is a substituted imidazolyl, substituted 3-pyridinyl, substituted 4-pyridinyl or substituted triazolyl; R¹ is hydrogen; and the other variables are as described above.

In another embodiment of the present invention, a compound is represented by Formula (III), or an E or Z isomer thereof, syn or anti isomer thereof, an optically pure isomer thereof, or pharmaceutically acceptable salt thereof, wherein Y is nitrogen, and the other variables are as described above.

Non-limiting examples of compounds of Formula (III) include 3-(((6-(-2-(Dimethylamino)-1-(1H-imidazol-1-yl)butyl)benzo[d]thiazol-2-yl)amino)methyl)benzoic acid; 3-((6-(-2-(Dimethylamino)-1-(1H-imidazol-1-yl)butyl)benzo[d]thiazol-2-yl)amino)-2,2-dimethylpropanoic acid; 3-((6-(-2-(Dimethylamino)-1-(1H-imidazol-1-yl)butyl)benzo[d]thiazol-2-yl)amino)benzoic acid; 4-(((6-(-2-(Dimethylamino)-1-(1H-imidazol-1-yl)butyl)benzo[d]thiazol-2-yl)amino)methyl)benzoic acid; 4-((6-(-2-(Dimethylamino)-1-(1H-imidazol-1-yl)butyl)benzo[d]thiazol-2-yl)amino)benzoic acid; Isopropyl 3-(((6-(-2-(dimethylamino)-1-(1H-imidazol-1-yl)butyl)benzo[d]thiazol-2-yl)amino)methyl)benzoate; Isopropyl 4-(((6-(-2-(dimethylamino)-1-(1H-imidazol-1-yl)butyl)benzo[d]thiazol-2-yl)amino)methyl)benzoate; Ethyl 3-(((6-(2-(dimethylamino)-1-(1H-imidazol-1-yl)butyl)benzo[d]thiazol-2-yl)amino)methyl)benzoate; Methyl 3-(((6-(-2-(dimethylamino)-1-(1H-imidazol-1-yl)butyl)benzo[d]thiazol-2-yl)amino)methyl)benzoate; Isopropyl 3-((6-(2-(dimethylamino)-1-(1H-imidazol-1-yl)butyl)benzo[d]thiazol-2-yl)amino)-2,2-dimethylpropanoate; Methyl 3-((6-(-2-(dimethylamino)-1-(1H-imidazol-1-yl)butyl)benzo[d]thiazol-2-yl)amino)benzoate; Methyl 4-(((6-(-2-(dimethylamino)-1-(1H-imidazol-1-yl)butyl)benzo[d]thiazol-2-yl)amino)methyl)benzoate; Methyl 4-((6-(-2-(dimethylamino)-1-(1H-imidazol-1-yl)butyl)benzo[d]thiazol-2-yl)amino)benzoate; 1-((6-(-2-(Dimethylamino)-1-(1H-imidazol-1-yl)butyl)benzo[d]thiazol-2-yl)carbamoyl)cyclobutanecarboxylic acid; 1-((6-(-2-(Dimethylamino)-1-(1H-imidazol-1-yl)butyl)benzo[d]thiazol-2-yl)carbamoyl)cyclohexanecarboxylic acid; 1-((6-(-2-(Dimethylamino)-1-(1H-imidazol-1-yl)butyl)benzo[d]thiazol-2-yl)carbamoyl)cyclopentanecarboxylic acid; 3-((6-(-2-(Dimethylamino)-1-(1H-imidazol-1-yl)butyl)benzo[d]thiazol-2-yl)carbamoyl)benzoic acid; 5-((6-(-2-(Dimethylamino)-1-(1H-imidazol-1-yl)butyl)benzo[d]thiazol-2-yl)amino)-2,2-dimethyl-4-oxobutanoic acid; 4-((6-(-2-(Dimethylamino)-1-(1H-imidazol-1-yl)butyl)benzo[d]thiazol-2-yl)carbamoyl)benzoic acid; 1-((6-(-2-(Dimethyl amino)-1-(1H-imidazol-1-yl)butyl)benzo[d]thiazol-2-yl)carbamoyl)cyclopropanecarboxylic acid; 5-((6-(-2-(Dimethylamino)-1-(1H-imidazol-1-yl)butyl)benzo[d]thiazol-2-yl)amino)-2,2-dimethyl-5-oxopentanoic acid; Ethyl 1-((6-(-2-(dimethylamino)-1-(1H-imidazol-1-yl)butyl)benzo[d]thiazol-2-yl)carbamoyl)cyclobutanecarboxylate; Methyl 1-((6-(-2-(dimethylamino)-1-(1H-imidazol-1-yl)butyl)benzo[d]thiazol-2-yl)carbamoyl)cyclohexanecarboxylate; Methyl 1-((6-(-2-(dimethylamino)-1-(1H-imidazol-1-yl)butyl)benzo[d]thiazol-2-yl)carbamoyl)cyclopentanecarboxylate; Methyl 3-((6-(-2-(dimethylamino)-1-(1H-imidazol-1-yl)butyl)benzo[d]thiazol-2-yl)carbamoyl)benzoate; Methyl 4-((6-(2-(dimethylamino)-1-(1H-imidazol-1-yl)butyl)benzo[d]thiazol-2-yl)carbamoyl)benzoate; Methyl 1-((6-(-2-(dimethylamino)-1-(1H-imidazol-1-yl)butyl)benzo[d]thiazol-2-yl)carbamoyl)cyclopropanecarboxylate; Methyl 5-((6-(-2-(dimethylamino)-1-(1H-imidazol-1-yl)butyl)benzo[d]thiazol-2-yl)amino)-2,2-dimethyl-5-oxopentanoate; N-(6-(-2-(dimethylamino)-1-(1H-imidazol-1-yl)butyl)benzo[d]thiazol-2-yl)bicyclo[2.2.1]heptane-2-carboxamide; Methyl 3-((6-(2-(dimethylamino)-1-(1H-imidazol-1-yl)butyl)benzo[d]thiazol-2-yl)oxy)benzoate; Methyl 4-((6-(2-(dimethylamino)-1-(1H-imidazol-1-yl)butyl)benzo[d]thiazol-2-yl)oxy)benzoate; 3-((6-(2-(Dimethylamino)-1-(1H-imidazol-1-yl)butyl)benzo[d]thiazol-2-yl)oxy)benzoic acid; 4-((6-(2-(Dimethylamino)-1-(1H-imidazol-1-yl)butyl)benzo[d]thiazol-2-yl)oxy)benzoic acid; 2-Chloro-4-(6-(2-(dimethylamino)-1-(1H-imidazol-1-yl)butyl)benzo[d]thiazol-2-yl)benzoic acid; 2-Chloro-5-(6-(2-(dimethylamino)-1-(1H-imidazol-1-yl)butyl)benzo[d]thiazol-2-yl)benzoic acid; 3-(6-(2-(Dimethylamino)-1-(1H-imidazol-1-yl)butyl)benzo[d]thiazol-2-yl)-5-(trifluoromethyl)benzoic acid; 3-(6-(2-(Dimethylamino)-1-(1H-imidazol-1-yl)butyl)benzo[d]thiazol-2-yl)-5-fluorobenzoic acid; 3-(6-(2-(Dimethyl amino)-1-(1H-imidazol-1-yl)butyl)benzo[d]thiazol-2-yl)benzoic acid; 3-(6-(2-(Ethyl(methyl)amino)-1-(1H-imidazol-1-yl)butyl)benzo[d]thiazol-2-yl)benzoic acid; 3-(6-(2-Ethyl-1-(1H-imidazol-1-yl)butyl)benzo[d]thiazol-2-yl)benzoic acid; 4-(6-(1-(1H-1,2,4-Triazol-1-yl)butyl)benzo[d]thiazol-2-yl)benzoic acid; 4-(6-(1-(Pyridin-3-yl)butyl)benzo[d]thiazol-2-yl)benzoic acid; 4-(6-(2-(Dimethylamino)-1-(1H-imidazol-1-yl)butyl)benzo[d]thiazol-2-yl)benzoic acid; 4-(6-(2-(Ethyl(methyl)amino)-1-(1H-imidazol-1-yl)butyl)benzo[d]thiazol-2-yl)benzoic acid; 4-(6-(2-Ethyl-1-(1H-imidazol-1-yl)butyl)benzo[d]thiazol-2-yl)benzoic acid; 5-(6-(2-(Dimethylamino)-1-(1H-imidazol-1-yl)butyl)benzo[d]thiazol-2-yl)-2-fluorobenzoic acid; Methyl 2-chloro-4-(6-(2-(dimethylamino)-1-(1H-imidazol-1-yl)butyl)benzo[d]thiazol-2-yl)benzoate; Methyl 2-chloro-5-(6-(2-(dimethylamino)-1-(1H-imidazol-1-yl)butyl)benzo[d]thiazol-2-yl)benzoate; Methyl 3-(6-(2-(dimethylamino)-1-(1H-imidazol-1-yl)butyl)benzo[d]thiazol-2-yl)-5-(trifluoromethyl)benzoate; Methyl 3-(6-(2-(dimethylamino)-1-(1H-imidazol-1-yl)butyl)benzo[d]thiazol-2-yl)-5-fluorobenzoate; Methyl 3-(6-(2-(dimethylamino)-1-(1H-imidazol-1-yl)butyl)benzo[d]thiazol-2-yl)benzoate; Methyl 3-(6-(2-(ethyl(methyl)amino)-1-(1H-imidazol-1-yl)butyl)benzo[d]thiazol-2-yl)benzoate; Methyl 3-(6-(2-ethyl-1-(1H-imidazol-1-yl)butyl)benzo[d]thiazol-2-yl)benzoate; Methyl 4-(6-(1-(1H-1,2,4-triazol-1-yl)butyl)benzo[d]thiazol-2-yl)benzoate; Methyl 4-(6-(1-(pyridin-3-yl)butyl)benzo[d]thiazol-2-yl)benzoate; Methyl 4-(6-(2-(dimethyl amino)-1-(1H-imidazol-1-yl)butyl)benzo[d]thiazol-2-yl)benzoate; Methyl 4-(6-(2-(ethyl(methyl)amino)-1-(1H-imidazol-1-yl)butyl)benzo[d]thiazol-2-yl)benzoate; Methyl 4-(6-(2-ethyl-1-(1H-imidazol-1-yl)butyl)benzo[d]thiazol-2-yl)benzoate; Methyl 5-(6-(2-(dimethylamino)-1-(1H-imidazol-1-yl)butyl)benzo[d]thiazol-2-yl)-2-fluorobenzoate; 1-(2-(3-(2H-Tetrazol-5-yl)phenyl)benzo[d]thiazol-6-yl)-1-(1H-imidazol-1-yl)-N,N-dimethylbutan-2-amine; 1-(2-(4-(2H-Tetrazol-5-yl)phenyl)benzo[d]thiazol-6-yl)-1-(1H-imidazol-1-yl)-N,N-dimethylbutan-2-amine; methyl 2-chloro-4-(6-(2-ethyl-1-(1H-imidazol-1-yl)butyl)benzo[d]thiazol-2-yl)benzoate; methyl 2-chloro-5-(6-(2-ethyl-1-(1H-imidazol-1-yl)butyl)benzo[d]thiazol-2-yl)benzoate; 2-(4-(6-(2-ethyl-1-(1H-imidazol-1-yl)butyl)benzo[d]thiazol-2-yl)phenyl)acetic acid; methyl 2-(4-(6-(2-ethyl-1-(1H-imidazol-1-yl)butyl)benzo[d]thiazol-2-yl)phenyl)acetate; 2-chloro-4-(6-(2-ethyl-1-(1H-imidazol-1-yl)butyl)benzo[d]thiazol-2-yl)benzoic acid; 2-chloro-5-(6-(2-ethyl-1-(1H-imidazol-1-yl)butyl)benzo[d]thiazol-2-yl)benzoic acid; 6-(2-(Dimethylamino)-1-(1H-imidazol-1-yl)butyl)benzo[d]thiazole-2-carboxylic acid; 6-(2-Ethyl-1-(1H-imidazol-1-yl)butyl)benzo[d]thiazole-2-carboxylic acid; Methyl 6-(2-(dimethylamino)-1-(1H-imidazol-1-yl)butyl)benzo[d]thiazole-2-carboxylate; Methyl 6-(2-(dimethylamino)-1-(1H-imidazol-1-yl)butyl)benzo[d]thiazole-2-carbimidate; Ethyl(6-(2-(dimethylamino)-1-(1H-imidazol-1-yl)butyl)benzo[d]thiazol-2-yl)carbamate; 6-(2-(Dimethylamino)-1-(1H-imidazol-1-yl)butyl)benzo[d]thiazole-2-carboxamide; N-(6-(2-(ethyl(methyl)amino)-1-(1H-imidazol-1-yl)butyl)benzo[d]thiazol-2-yl)acetamide; 1-(6-(2-(Dimethylamino)-1-(1H-imidazol-1-yl)butyl)benzo[d]thiazol-2-yl)urea; 1-(6-(2-(Dimethylamino)-1-(1H-imidazol-1-yl)butyl)benzo[d]thiazol-2-yl)-2,2,2-trifluoroethanone; 1-(6-(2-Ethyl-1-(1H-imidazol-1-yl)butyl)benzo[d]thiazol-2-yl)-2,2,2-trifluoro ethanone; 1-(1H-Imidazol-1-yl)-N,N-dimethyl-1-(2-phenylbenzo[d]thiazol-6-yl)butan-2-amine; 1-(2-Bromobenzo[d]thiazol-6-yl)-1-(1H-imidazol-1-yl)-N,N-dimethylbutan-2-amine; 1-(2-Bromobenzo[d]thiazol-6-yl)-N-ethyl-1-(1H-imidazol-1-yl)-N-methylbutan-2-amine; 1-(2-Chlorobenzo[d]thiazol-6-yl)-1-(1H-imidazol-1-yl)-N,N-dimethylbutan-2-amine; 1-(2-Chlorobenzo[d]thiazol-6-yl)-N-ethyl-1-(1H-imidazol-1-yl)-N-methylbutan-2-amine; 1-(Benzo[d]thiazol-6-yl)-1-(1H-imidazol-1-yl)-N,N-dimethylbutan-2-amine; 6-(2-(Dimethylamino)-1-(1H-imidazol-1-yl)butyl)benzo[d]thiazol-2-amine; 6-(2-(Ethyl(methyl)amino)-1-(1H-imidazol-1-yl)butyl)benzo[d]thiazol-2-amine; 6-(2-(Dimethylamino)-1-(1H-imidazol-1-yl)butyl)benzo[d]thiazol-2 (3H)-one; 6-(2-Ethyl-1-(1H-imidazol-1-yl)butyl)benzo[d]thiazol-2 (3H)-one; 1-(1H-Imidazol-1-yl)-1-(2-methoxybenzo[d]thiazol-6-yl)-N,N-dimethylbutan-2-amine; 1-(1H-Imidazol-1-yl)-N,N-dimethyl-1-(2-methylbenzo[d]thiazol-6-yl)butan-2-amine; 6-(2-Ethyl-1-(1H-imidazol-1-yl)butyl)-2-methoxybenzo[d]thiazole; 6-(2-Ethyl-1-(1H-imidazol-1-yl)butyl)-2-methylbenzo[d]thiazole; 6-(2-Ethyl-1-(1H-imidazol-1-yl)butyl)benzo[d]thiazol-2-amine; 2-Bromo-6(2-ethyl-1-(1H-imidazol-1-yl)butyl)benzo[d]thiazole; 6-(2-Ethyl-1-(1H-imidazol-1-yl)butyl)-2-phenylbenzo[d]thiazole; 2-(6-(2-(Dimethylamino)-1-(1H-imidazol-1-yl)butyl)benzo[d]thiazol-2-yl)acetic acid; 6-(1-(1H-1,2,4-triazol-1-yl)butyl)-2-bromobenzo[d]thiazole and 2-(6-(2-Ethyl-1-(1H-imidazol-1-yl)butyl)benzo[d]thiazol-2-yl)acetic acid.

In another embodiment, the CYP26 inhibitor of the synergistic composition is represented by Formula (IV):

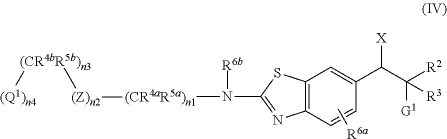

or an E or Z isomer thereof, syn or anti isomer thereof, an optically pure isomer thereof, or pharmaceutically acceptable salt thereof, wherein:
X is an unsaturated heterocycle selected from pyrrolyl, pyrazolyl, imidazolyl, triazolyl, benzimidazolyl, benzotriazolyl, tetrazolyl, thiazole, 3-pyridinyl or 4-pyridinyl, any of which is optionally substituted with one or more independent R⁶⁶ substituents;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthio C₂₋₁₀alkenyl, C₁₋₁₀alkylthio C₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, mono C₁₋₆alkylaminocarbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR⁷⁷—SO₂NR⁷¹R⁸¹ or —CONR⁷¹R⁸¹ substituents;
G¹ is hydrogen, C₀₋₁₀alkyl, C₂₋₁₀alkenyl, C₂₋₁₀alkynyl, —OR⁷², —SR⁷², —NR⁷²R⁸² (R⁹)ₙ₅, or G¹ and R³ taken together with the carbon atom to which they are attached forma 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸² (R⁹)ₙ₅, R⁷² and R⁸² taken together with the nitrogen atom to which they are attached form a 3-10 membered heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with one or more independent halo, cyano, hydroxy, nitro, R⁶⁸, C₁₋₁₀alkoxy, —COOR⁷⁴, —(C═O)N(R⁷⁴)—, —NR⁷⁴CONR⁷⁴R⁷⁵, —NR⁷⁴COOR⁷⁵, —SO₂N^{R6}R⁸⁶, —NR⁷⁴SO₂NR⁷⁴R⁷⁵, or —NR⁷⁶R⁸⁶ substituents;
Q¹ is C₀₋₆alkyl, cycloC₃₋₈alkyl, bridged bicycloalkyl, —OR⁷⁵, —COR⁷⁴, —NR⁷⁵R⁸⁵(R⁹⁵)ₙ₆, —CO₂R⁷⁵, —CONR⁷⁵R⁸⁵, —(C═S)OR⁷⁵, —(C═O)SR⁷⁵, —NO₂, —CN, halo, —S(O)ₙ₆R⁷⁵, —SO₂NR⁷⁵R⁸⁵, —NR⁷⁵(C═NR⁷⁷⁵)NR⁷⁷⁷⁵R⁸⁵, —NR⁷⁵(C═NR⁷⁷⁵)OR⁷⁷⁷⁵, —NR⁷⁵(C═NR⁷⁷⁵)SR⁷⁷⁷⁵, —O(C═O)OR⁷⁵, —O(C═O)NR⁷⁵R⁸⁵, —O(C═O)SR⁷⁵, —S(C═O)OR⁷⁵, —S(C═O)NR⁷⁵R⁸⁵, —S(C═O)SR⁷⁵, —NR⁷⁵(C═O)NR⁷⁷⁵R⁸⁵, or —NR⁷⁵(C═S)NR⁷⁷⁵R⁸⁵; in the case of —NR⁷⁵R⁸⁵(R⁹⁵)ₙ₆, R⁷⁵ and R⁸⁵ taken together with the nitrogen atom to which they are attached form a 3-10 membered heterocyclic saturated ring, or heterocyclic unsaturated ring, any of which is optionally substituted with one or more independent halo, cyano, hydroxy, nitro, C₁₋₁₀alkoxy, —SO₂N^{R6}R⁸⁶ or —NR⁷⁶R⁸⁶ substituents;
R^{4a}, R^{4b}, R^{4c}, R^{5a}, R^{5b} and R^{5c} are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono(C₁₋₆alkyl)amino C₁₋₆alkyl, di(C₁₋₆alkyl)aminoC₁₋₆alkyl, mono(aryl)amino C₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or R^{4a} with R^{5a}, or R^{4b} with R^{5b}, or R^{4c} with R^{5c}, taken together with the respective carbon atom to which they are attached, form a carbonyl or 3-10 membered saturated or unsaturated monocyclic or polycyclic ring, wherein said ring is optionally substituted with R⁶⁹; or R^{4a} with R^{5a}, or R^{4b} with R^{5b}, or R^{4c} with R^{5c}, taken together with the respective carbon atom to which they are attached, form a 3-10 membered saturated or unsaturated monoheterocyclic or polyheterocyclic ring, wherein said ring is optionally substituted with R⁶⁹;
R^{6a}, R^{6b}, R⁶⁶, R⁶⁷, R⁶⁸, and R⁶⁹ are each independently hydrogen, halo, —OR⁷⁷—SH, —NR⁷⁷R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, C₁₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)amino C₁₋₆alkyl, di(C₁₋₆alkyl)aminoC₁₋₆alkyl, mono(aryl)amino C₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered mono or polycyclic saturated ring, mono or polycyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R⁸⁵, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynmyl, C₁₋₁₀alkylthioC₁₋₁₀alkyl, C₁₋₁₀alkylthio C₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl) or —N(C₀₋₄alkyl) (C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl) (C₀₋₄alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CON(C₀₋₄ alkyl)(C₀₋₄alkyl), —SO₂N(C₀₋₄ alkyl)(C₀₋₄alkyl) or —N(C₀₋₄alkyl) (C₀₋₄alkyl) substituents; and
n1, n2, n3, n4, n5, n6, and n7 are each independently equal to 0, 1 or 2.

In another embodiment, CYP26 inhibitors useful in the synergistic composition are represented by Formula (IV-A):

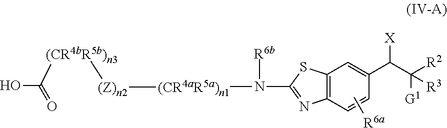

or an E or Z isomer thereof, syn or anti isomer thereof, an optically pure isomer thereof, or pharmaceutically acceptable salt thereof, and wherein:
X is optionally substituted imidazolyl, triazolyl, 3-pyridinyl or 4-pyridinyl;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenyl carbonyl, C₂₋₁₀alkynylcarbonyl, C₁₋₁₀alkoxycarbonyl, C₁₋₁₀alkoxycarbonylC₁₋₁₀alkyl, mono C₁₋₆alkylaminocarbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
   or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR⁷⁷—SO₂NR⁷¹R⁸¹ or —CONR⁷¹R⁸¹ substituents;
   G¹ is hydrogen, C₀₋₁₀alkyl, C₂₋₁₀alkenyl, C₂₋₁₀alkynyl, —OR⁷², —SR⁷², —NR⁷²R⁸²(R⁹)ₙ₅, or G¹ and R³ taken together with the carbon atom to which they are attached forma 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
   Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with one or more independent halo, cyano, hydroxy, nitro, R⁶⁸, C₁₋₁₀alkoxy, —COOR⁷⁴, —(C═O)N(R⁷⁴)—, —NR⁷⁴CONR⁷⁴R⁷⁵, —NR⁷⁴COOR⁷⁵, —SO₂N^{R6}R⁸⁶, —NR⁷⁴SO₂NR⁷⁴R⁷⁵, or —NR⁷⁶R⁸⁶ substituents; R^{4a}, R^{4b}, R^{4c}, R^{5a}, R^{5b}, and R^{5c} are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthio C₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono(C₁₋₆alkyl)amino C₁₋₆alkyl, di(C₁₋₆alkyl)aminoC₁₋₆alkyl, mono(aryl)amino C₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or R^{4a} with R^{5a}, or R^{4b} with R^{5b}, or R^{4c} with R^{5c}, taken together with the respective carbon atom to which they are attached, form a carbonyl or 3-10 membered saturated or unsaturated monocyclic or polycyclic ring, wherein said ring is optionally substituted with R⁶⁹; or R^{4a} with R^{5a}, or R^{4b} with R^{5b}, or R^{4c} with R^{5c}, taken together with the respective carbon atom to which they are attached, form a 3-10 membered saturated or unsaturated monoheterocyclic or polyheterocyclic ring, wherein said ring is optionally substituted with R⁶⁹;
   R^{6a}, R^{6b}, R⁶⁶, R⁶⁷, R⁶⁸, and R⁶⁹ are each independently hydrogen, halo, —OR⁷⁷—SH, —NR⁷⁷R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, C₁₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)amino C₁₋₆alkyl, di(C₁₋₆alkyl)aminoC₁₋₆alkyl, mono(aryl)amino C₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered mono or polycyclic saturated ring, mono or polycyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R⁸⁵, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynmyl, C₁₋₁₀alkylthioC₁₋₁₀alkyl, C₁₋₁₀alkylthio C₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl) or —N(C₀₋₄alkyl) (C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₁₀alkyl), —SO₂N(C₀₋₄ alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl) (C₀₋₄alkyl) substituents; or mono(C₁₋₆alkyl)aminoC₁₋₆alkyl, di(C₁₋₆alkyl)aminoC₁₋₆ alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CON(C₀₋₄ alkyl)(C₀₋₄alkyl), —SO₂N(C₀₋₄alkyl)(C₀₋₄alkyl) or —N(C₀₋₄alkyl) (C₀₋₄alkyl) substituents; and
n1, n2, n3, n5 and n7 are each independently equal to 0, 1 or 2.

In another embodiment, a compound is represented by Formula (I), or an E or Z isomer thereof, syn or anti isomer thereof, an optically pure isomer thereof, or pharmaceutically acceptable salt thereof, wherein Y is NR^{6b}CO—, and the other variables are as described above.

In another embodiment, CYP26 inhibitors comprising the synergistic composition are represented by Formula (V):

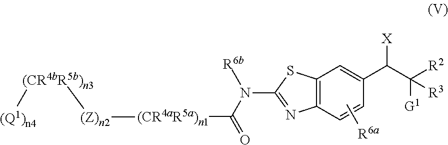

or an E or Z isomer thereof, syn or anti isomer thereof, an optically pure isomer thereof, or pharmaceutically acceptable salt thereof, and wherein:
X is an unsaturated heterocycle selected from pyrrolyl, pyrazolyl, imidazolyl, triazolyl, benzimidazolyl, benzotriazolyl, tetrazolyl, thiazole, 3-pyridinyl or 4-pyridinyl, any of which is optionally substituted with one or more independent R⁶⁶ substituents;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, mono C₁₋₆alkylaminocarbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR⁷⁷, —SO₂NR⁷¹R⁸¹ or —CONR⁷¹R⁸¹ substituents;
G¹ is hydrogen, C₀₋₁₀alkyl, C₂₋₁₀alkenyl, C₂₋₁₀alkynyl, —OR⁷², —SR⁷², —NR⁷²R⁸²(R⁹)ₙ₅, or G¹ and R³ taken together with the carbon atom to which they are attached forma 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with one or more independent halo, cyano, hydroxy, nitro, R⁶⁸, C₁₋₁₀alkoxy, —COOR⁷⁴, —(C═O)N(R⁷⁴)—, —NR⁷⁴CONR⁷⁴R⁷⁵, —NR⁷⁴COOR⁷⁵, —SO₂N^{R6}R⁸⁶, —NR⁷⁴SO₂NR⁷⁴R⁷⁵, or —NR⁷⁶R⁸⁶ substituents;
Q¹ is C₀₋₆alkyl, cycloC₃₋₈alkyl, bridged bicycloalkyl, —OR⁷⁵, —COR⁷⁴, —NR⁷⁵R⁸⁵(R⁹⁵)ₙ₆, —CO₂R⁷⁵, —CONR⁷⁵R⁸⁵, —(C═S)OR⁷⁵, —(C═O)SR⁷⁵, —NO₂, —CN, halo, —S(O)ₙ₆R⁷⁵, —SO₂NR⁷⁵R⁸⁵, —NR⁷⁵(C═NR⁷⁷⁵)NR⁷⁷⁷⁵R⁸⁵, —NR⁷⁵(C═NR⁷⁷⁵)OR⁷⁷⁷⁵, —NR⁷⁵(C═NR⁷⁷⁵)SR⁷⁷⁷⁵, —O(C═O)OR⁷⁵, —O(C═O)NR⁷⁵R⁸⁵, —O(C═O)SR⁷⁵, —S(C═O)OR⁷⁵, —S(C═O)NR⁷⁵R⁸⁵, —S(C═O)SR⁷⁵, —NR⁷⁵(C═O)NR⁷⁷⁵R⁸⁵, or —NR⁷⁵(C═S)NR⁷⁷⁵R⁸⁵; in the case of—NR⁷⁵R⁸⁵(R⁹⁵)ₙ₆, R⁷⁵ and R⁸⁵ taken together with the nitrogen atom to which they are attached form a 3-10 membered heterocyclic saturated ring, or heterocyclic unsaturated ring, any of which is optionally substituted with one or more independent halo, cyano, hydroxy, nitro, C₁₋₁₀alkoxy, —SO₂N^{R6}R⁸⁶ or —NR⁷⁶R⁸⁶ substituents;
R^{4a}, R^{4b}, R^{4c}, R^{5a}, R^{5b} and R^{5c} are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or R^{4a} with R^{5a}, or R^{4b} with R^{5b}, or R^{4c} with R^{5c}, taken together with the respective carbon atom to which they are attached, form a carbonyl or 3-10 membered saturated or unsaturated monocyclic or polycyclic ring, wherein said ring is optionally substituted with R⁶⁹; or R^{4a} with R^{5a}, or R^{4b} with R^{5b}, or R^{4c} with R^{5c}, taken together with the respective carbon atom to which they are attached, form a 3-10 membered saturated or unsaturated monoheterocyclic or polyheterocyclic ring, wherein said ring is optionally substituted with R⁶⁹;
R^{6a}, R^{6b}, R⁶⁶, R⁶⁷, R⁶⁸, and R⁶⁹ are each independently hydrogen, halo, —OR⁷⁷, —SH, —NR⁷⁷R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, C₁₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthio C₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)amino C₁₋₆alkyl, di(C₁₋₆alkyl)aminoC₁₋₆alkyl, mono(aryl)amino C₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered mono or polycyclic saturated ring, mono or polycyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R⁸⁵, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynmyl, C₁₋₁₀alkylthioC₁₋₁₀alkyl, C₁₋₁₀alkylthio C₂₋₁₀alkenyl,

C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl) or —N(C₀₋₄alkyl) (C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl) (C₀₋₄alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CON(C₀₋₄ alkyl)(C₀₋₄alkyl), —SO₂N(C₀₋₄ alkyl)(C₀₋₄alkyl) or —N(C₀₋₄alkyl) (C₀₋₄alkyl) substituents; and
n1, n2, n3, n4, n5, n6, and n7 are each independently equal to 0, 1 or 2.

In another embodiment, CYP26 inhibitors comprising the synergistic composition are represented by Formula (V-A):

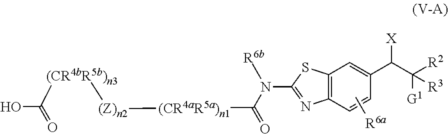

or an E or Z isomer thereof, syn or anti isomer thereof, an optically pure isomer thereof, or pharmaceutically acceptable salt thereof, and wherein:
X is optionally substituted imidazolyl, triazolyl, 3-pyridinyl or 4-pyridinyl;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenyl carbonyl, C₂₋₁₀alkynylcarbonyl, C₁₋₁₀alkoxycarbonyl, C₁₋₁₀alkoxycarbonylC₁₋₁₀alkyl, mono C₁₋₆alkylaminocarbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR⁷⁷, —SO₂NR⁷¹R⁸¹ or —CONR⁷¹R⁸¹ substituents;
G¹ is hydrogen, C₀₋₁₀alkyl, C₂₋₁₀alkenyl, C₂₋₁₀alkynyl, —OR⁷², —SR⁷², —NR⁷²R⁸²(R⁹)ₙ₅, or G¹ and R³ taken together with the carbon atom to which they are attached forma 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with one or more independent halo, cyano, hydroxy, nitro, R⁶⁸, C₁₋₁₀alkoxy, —COOR⁷⁴, —(C═O)N(R⁷⁴)—, —NR⁷⁴CONR⁷⁴R⁷⁵, —NR⁷⁴COOR⁷⁵, —SO₂N^{R6}R⁸⁶, —NR⁷⁴SO₂NR⁷⁴R⁷⁵, or —NR⁷⁶R⁸⁶ substituents; R^{4a}, R^{4b}, R^{4c}, R^{5a}, R^{5b} and R^{5a} are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono(C₁₋₆alkyl)amino C₁₋₆alkyl, di(C₁₋₆alkyl)aminoC₁₋₆alkyl, mono(aryl)amino C₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or R^{4a} with R^{5a}, or R^{4b} with R^{5b}, or R^{4c} with R^{5c}, taken together with the respective carbon atom to which they are attached, form a carbonyl or 3-10 membered saturated or unsaturated monocyclic or polycyclic ring, wherein said ring is optionally substituted with R⁶⁹; or R^{4a} with R^{5a}, or R^{4b} with R^{5b}, or R^{4c} with R^{5c}, taken together with the respective carbon atom to which they are attached, form a 3-10 membered saturated or unsaturated monoheterocyclic or polyheterocyclic ring, wherein said ring is optionally substituted with R⁶⁹;
   R^{6a}, R^{6b}, R⁶⁶, R⁶⁷, R⁶⁸, and R⁶⁹ are each independently hydrogen, halo, —OR⁷⁷, —SH, —NR⁷⁷R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, C₁₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₁₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)amino C₁₋₆alkyl, di(C₁₋₆alkyl)aminoC₁₋₆alkyl, mono(aryl)amino C₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered mono or polycyclic saturated ring, mono or polycyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R⁸⁵, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynmyl, C₁₋₁₀alkylthioC₁₋₁₀alkyl, C₁₋₁₀alkylthio C₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl) or —N(C₀₋₄alkyl) (C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₁₀alkyl), —SO₂N(C₀₋₄ alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₄alkyl), —SO₂N(C₀₋₄ alkyl)(C₀₋₄alkyl) or —N(C₀₋₄alkyl) (C₀₋₄alkyl) substituents; or mono(C₁₋₆alkyl)aminoC₁₋₆alkyl, di(C₁₋₆alkyl)aminoC₁₋₆ alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CON(C₀₋₄ alkyl)(C₀₋₄alkyl), —SO₂N(C₀₋₄ alkyl)(C₀₋₄alkyl) or —N(C₀₋₄alkyl) (C₀₋₄alkyl) substituents; and
n1, n2, n3, n5, and n7 are each independently equal to 0, 1 or 2.
   In another embodiment, a compound is represented by Formula (I), or an E or Z isomer thereof, syn or anti isomer thereof, an optically pure isomer thereof, or pharmaceutically acceptable salt thereof, wherein Y is oxygen, and the other variables are as described above.

In another embodiment, CYP26 inhibitors comprising the synergistic composition are represented by Formula (VI):

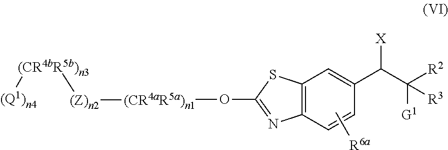

or an E or Z isomer thereof, syn or anti isomer thereof, an optically pure isomer thereof, or pharmaceutically acceptable salt thereof, and wherein;
X is an unsaturated heterocycle selected from pyrrolyl, pyrazolyl, imidazolyl, triazolyl, benzimidazolyl, benzotriazolyl, tetrazolyl, thiazole, 3-pyridinyl or 4-pyridinyl, any of which is optionally substituted with one or more independent R⁶⁶ substituents;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, mono C₁₋₆alkylaminocarbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR⁷⁷, —SO₂NR⁷¹R⁸¹ or —CONR⁷¹R⁸¹ substituents;
G¹ is hydrogen, C₀₋₁₀alkyl, C₂₋₁₀alkenyl, C₂₋₁₀alkynyl, —OR⁷², —SR⁷², —NR⁷²R⁸²(R⁹)ₙ₅, or G¹ and R³ taken together with the carbon atom to which they are attached forma 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸² (R⁹)ₙ₅, R⁷² and R⁸² taken together with the nitrogen atom to which they are attached form a 3-10 membered heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with one or more independent halo, cyano, hydroxy, nitro, R⁶⁸, C₁₋₁₀alkoxy, —COOR⁷⁴, —(C═O)N(R⁷⁴)—, —NR⁷⁴CONR⁷⁴R⁷⁵, —NR⁷⁴COOR⁷⁵, —SO₂N^{R6}R⁸⁶, —NR⁷⁴SO₂NR⁷⁴R⁷⁵, or —NR⁷⁶R⁸⁶ substituents;
   Q¹ is C₀₋₆alkyl, cycloC₃₋₈alkyl, bridged bicycloalkyl, —OR⁷⁵, —COR⁷⁴, —NR⁷⁵R⁸⁵(R⁹⁵)ₙ₆, —CO₂R⁷⁵, —CONR⁷⁵R⁸⁵, —(C═S)OR⁷⁵, —(C═O)SR⁷⁵, —NO₂, —CN, halo, —S(O)ₙ₆R⁷⁵, —SO₂NR⁷⁵R⁸⁵, —NR⁷⁵(C═NR⁷⁷⁵)NR⁷⁷⁷⁵R⁸⁵, —NR⁷⁵(C═NR⁷⁷⁵)OR⁷⁷⁷⁵, —NR⁷⁵(C═NR⁷⁷⁵)SR⁷⁷⁷⁵, —O(C═O)OR⁷⁵, —O(C═O)NR⁷⁵R⁸⁵, —O(C═O)SR⁷⁵, —S(C═O)OR⁷⁵, —S(C═O)NR⁷⁵R⁸⁵, —S(C═O)SR⁷⁵, —NR⁷⁵(C═O)NR⁷⁷⁵R⁸⁵, or —NR⁷⁵(C═S)NR⁷⁷⁵R⁸⁵; in the case of —NR⁷⁵R⁸⁵(R⁹⁵)ₙ₆, R⁷⁵ and R⁸⁵ taken together with the nitrogen atom to which they are attached form a 3-10 membered heterocyclic saturated ring, or heterocyclic unsaturated ring, any of which is optionally substituted with one or more independent halo, cyano, hydroxy, nitro, C₁₋₁₀alkoxy, —SO₂N^{R6}R⁸⁶ or —NR⁷⁶R⁸⁶ substituents;
R^{4a}, R^{4b}, R^{4c}, R^{5a}, R^{5b} and R^{5c} are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono(C₁₋₆alkyl)amino C₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or R^{4a} with R^{5a}, or R^{4b} with R^{5b}, or R^{4c} with R^{5c}, taken together with the respective carbon atom to which they are attached, form a carbonyl or 3-10 membered saturated or unsaturated monocyclic or polycyclic ring, wherein said ring is optionally substituted with R⁶⁹; or R^{4a} with R^{5a}, or R^{4b} with R^{5b}, or R^{4c} with R^{5c}, taken together with the respective carbon atom to which they are attached, form a 3-10 membered saturated or unsaturated monoheterocyclic or polyheterocyclic ring, wherein said ring is optionally substituted with R⁶⁹;
R^{6a}, R⁶⁶, R⁶⁷, R⁶⁸, and R⁶⁹ are each independently hydrogen, halo, —OR⁷⁷, —SH, —NR⁷⁷R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, C₁₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthio C₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)amino C₁₋₆alkyl, di(C₁₋₆alkyl)amino C₁₋₆alkyl, mono(aryl)amino C₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered mono or polycyclic saturated ring, mono or polycyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
   R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R⁸⁵, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynmyl, C₁₋₁₀alkylthioC₁₋₁₀alkyl, C₁₋₁₀alkylthio C₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl) or —N(C₀₋₄alkyl) (C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl) (C₀₋₄alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₄alkyl), —SO₂N(C₀₋₄ alkyl)(C₀₋₄alkyl) or —N(C₀₋₄alkyl) (C₀₋₄alkyl) substituents; and
n1, n2, n3, n4, n5, n6, and n7 are each independently equal to 0, 1 or 2.

In another embodiment, CYP26 inhibitors comprising the synergistic composition are represented by Formula (VI-A):

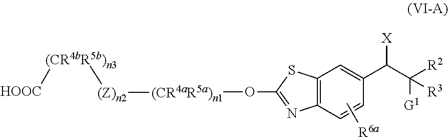

or an E or Z isomer thereof, syn or anti isomer thereof, an optically pure isomer thereof, or pharmaceutically acceptable salt thereof, and wherein;
X is optionally substituted imidazolyl, triazolyl, 3-pyridinyl or 4-pyridinyl;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₁₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, mono C₁₋₆alkylaminocarbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
   or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR⁷⁷, —SO₂NR⁷¹R⁸¹ or —CONR⁷¹R⁸¹ substituents;
   G¹ is hydrogen, C₀₋₁₀alkyl, C₂₋₁₀alkenyl, C₂₋₁₀alkynyl, —OR⁷², —SR⁷², —NR⁷²R⁸²(R⁹)ₙ₅, or G¹ and R³ taken together with the carbon atom to which they are attached forma 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with one or more independent halo, cyano, hydroxy, nitro, R⁶⁸, C₁₋₁₀alkoxy, —COOR⁷⁴, —(C═O)N(R⁷⁴)—, —NR⁷⁴CONR⁷⁴R⁷⁵, —NR⁷⁴COOR⁷⁵, —SO₂N^{R6}R⁸⁶, —NR⁷⁴SO₂NR⁷⁴R⁷⁵, or —NR⁷⁶R⁸⁶ substituents; R^{4a}, R^{4b}, R^{4c}, R^{5a}, R^{5b} and R^{5a} are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthio C₂₋₁₀alkenyl, C₁₋₁₀alkylthio C₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono(C₁₋₆alkyl)amino C₁₋₆alkyl, di(C₁₋₆alkyl)aminoC₁₋₆alkyl, mono(aryl)amino C₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or R^{4a} with R^{5a}, or R^{4b} with R^{5b}, or R^{4c} with R^{5c}, taken together with the respective carbon atom to which they are attached, form a carbonyl or 3-10 membered saturated or unsaturated monocyclic or polycyclic ring, wherein said ring is optionally substituted with R⁶⁹; or R^{4a} with R^{5a}, or R^{4b} with R^{5b}, or R^{4c} with R^{5c}, taken together with the respective carbon atom to which they are attached, form a 3-10 membered saturated or unsaturated monoheterocyclic or polyheterocyclic ring, wherein said ring is optionally substituted with R⁶⁹;
R^{6a}, R⁶⁶, R⁶⁷, R⁶⁸, and R⁶⁹ are each independently hydrogen, halo, —OR⁷⁷, —SH, —NR⁷⁷R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)amino C₁₋₆alkyl, di(C₁₋₆alkyl)amino C₁₋₆alkyl, mono(aryl)amino C₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered mono or polycyclic saturated ring, mono or polycyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R⁸⁵, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynmyl, C₁₋₁₀alkylthioC₁₋₁₀alkyl, C₁₋₁₀alkylthio C₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkyl carbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl) or —N(C₀₋₄alkyl) (C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl) (C₀₋₄alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CON(C₀₋₄ alkyl)(C₀₋₄alkyl), —SO₂N(C₀₋₄ alkyl)(C₀₋₄alkyl) or —N(C₀₋₄alkyl) (C₀₋₄alkyl) substituents; and
n1, n2, n3, n5, and n7 are each independently equal to 0, 1 or 2.

In another embodiment, a compound is represented by Formula (I), or an E or Z isomer thereof, syn or anti isomer thereof, an optically pure isomer thereof, or pharmaceutically acceptable salt thereof, wherein Y is aryl or heteroaryl, and the other variables are as described above.

In another embodiment, CYP26 inhibitors comprising the synergistic composition are represented by Formula (VII):

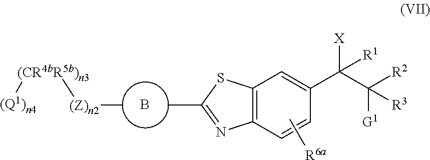

or an E or Z isomer thereof, syn or anti isomer thereof, an optically pure isomer thereof, or pharmaceutically acceptable salt thereof, wherein:
X is an unsaturated heterocycle selected from pyrrolyl, pyrazolyl, imidazolyl, triazolyl, benzimidazolyl, benzotriazolyl, tetrazolyl, thiazole, 3-pyridinyl or 4-pyridinyl, any of which is optionally substituted with one or more independent R⁶⁶ substituents;
R¹ is hydrogen, C₀₋₆alkyl, —OR⁷, —SR⁷, or —NR⁷R⁸;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, mono C₁₋₆alkylaminocarbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
   or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR⁷⁷, —SO₂NR⁷¹R⁸¹ or —CONR⁷¹R⁸¹ substituents;
G¹ is hydrogen, C₀₋₁₀alkyl, C₂₋₁₀alkenyl, C₂₋₁₀alkynyl, —OR⁷², —SR⁷², —NR⁷²R⁸²(R⁹)ₙ₅, or G¹ and R³ taken together with the carbon atom to which they are attached forma 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
B is aryl or heteroaryl which is optionally substituted with one or more independent halo, —OR⁷⁷—SR⁷⁷, —NR⁷⁷R⁸⁸(R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthio C₂₋₁₀alkenyl, C₀₋₁₀alkylthio C₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with one or more independent halo, cyano, hydroxy, nitro, R⁶⁸, C₁₋₁₀alkoxy, —COOR⁷⁴, —(C═O)N(R⁷⁴)—, —NR⁷⁴CONR⁷⁴R⁷⁵, —NR⁷⁴COOR⁷⁵, —SO₂N^{R6}R⁸⁶, —NR⁷⁴SO₂NR⁷⁴R⁷⁵, or —NR⁷⁶R⁸⁶ substituents;
Q¹ is C₀₋₆alkyl, cycloC₃₋₈alkyl, bridged bicycloalkyl, —OR⁷⁵, —COR⁷⁴, —NR⁷⁵R⁸⁵(R⁹⁵)ₙ₆, —CO₂R⁷⁵, —CONR⁷⁵R⁸⁵, —(C═S)OR⁷⁵, —(C═O)SR⁷⁵, —NO₂, —CN, halo, —S(O)ₙ₆R⁷⁵, —SO₂NR⁷⁵R⁸⁵, —NR⁷⁵(C═NR⁷⁷⁵)NR⁷⁷⁷⁵R⁸⁵, —NR⁷⁵(C═NR⁷⁷⁵)OR⁷⁷⁷⁵, —NR⁷⁵(C═NR⁷⁷⁵)SR⁷⁷⁷⁵, —O(C═O)OR⁷⁵, —O(C═O)NR⁷⁵R⁸⁵, —O(C═O)SR⁷⁵, —S(C═O)OR⁷⁵, —S(C═O)NR⁷⁵R⁸⁵, —S(C═O)SR⁷⁵, —NR⁷⁵(C═O)NR⁷⁷⁵R⁸⁵, or —NR⁷⁵(C═S)NR⁷⁷⁵R⁸⁵; in the case of—NR⁷⁵R⁸⁵(R⁹⁵)ₙ₆, R⁷⁵ and R⁸⁵ taken together with the nitrogen atom to which they are attached form a 3-10 membered heterocyclic saturated ring, or heterocyclic unsaturated ring, any of which is optionally substituted with one or more independent halo, cyano, hydroxy, nitro, C₁₋₁₀alkoxy, —SO₂N^{R6}R⁸⁶ or —NR⁷⁶R⁸⁶ substituents;
R^{4a}, R^{4b}, R^{4c}, R^{5a}, R^{5b} and R^{5c} are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthio C₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono(C₁₋₆alkyl)amino C₁₋₆alkyl, di(C₁₋₆alkyl)amino C₁₋₆alkyl, mono(aryl)amino C₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or R^{4a} with R^{5a}, or R^{4b} with R^{5b}, or R^{4c} with R^{5c}, taken together with the respective carbon atom to which they are attached, form a carbonyl or 3-10 membered saturated or unsaturated monocyclic or polycyclic ring, wherein said ring is optionally substituted with R⁶⁹; or R^{4a} with R^{5a}, or R^{4b} with R^{5b}, or R^{4c} with R^{5c}, taken together with the respective carbon atom to which they are attached, form a 3-10 membered saturated or unsaturated monoheterocyclic or polyheterocyclic ring, wherein said ring is optionally substituted with R⁶⁹;
R^{6a}, R⁶⁶, R⁶⁷, R⁶⁸, and R⁶⁹ are each independently hydrogen, halo, —OR⁷⁷, —SH, —NR⁷⁷R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)amino C₁₋₆alkyl, di(C₁₋₆alkyl)amino C₁₋₆alkyl, mono(aryl)amino C₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered mono or polycyclic saturated ring, mono or polycyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R⁸⁵, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynmyl, C₁₋₁₀alkylthioC₁₋₁₀alkyl, C₁₋₁₀alkylthio C₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl) or —N(C₀₋₄alkyl) (C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₁₀alkyl), —SO₂N(C₀₋₄ alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₄alkyl), —SO₂N(C₀₋₄ alkyl)(C₀₋₄alkyl) or —N(C₀₋₄alkyl) (C₀₋₄alkyl) substituents; or mono(C₁₋₆alkyl)aminoC₁₋₆alkyl, di(C₁₋₆alkyl)aminoC₁₋₆ alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CON(C₀₋₄ alkyl)(C₀₋₄alkyl), —SO₂N(C₀₋₄ alkyl)(C₀₋₄alkyl) or —N(C₀₋₄alkyl) (C₀₋₄alkyl) substituents; and
n2, n3, n4, n5, n6, and n7 are each independently equal to 0, 1 or 2.

In another embodiment, CYP26 inhibitors comprising the synergistic composition are represented by Formula (VII-A):

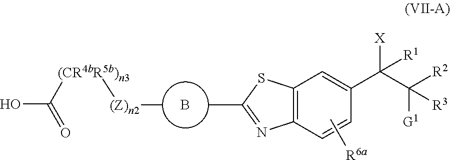

or an E or Z isomer thereof, syn or anti isomer thereof, an optically pure isomer thereof, or pharmaceutically acceptable salt thereof, wherein:
X is optionally substituted imidazolyl, triazolyl, benzimidazolyl, benzotriazolyl, 3-pyridinyl or 4-pyridinyl;
R¹ is hydrogen, C₀₋₆alkyl, —OR⁷, —SR⁷, or —NR⁷R⁸;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenyl carbonyl, C₂₋₁₀alkynylcarbonyl, C₁₋₁₀alkoxycarbonyl, C₁₋₁₀alkoxycarbonylC₁₋₁₀alkyl, mono C₁₋₆alkylaminocarbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR⁷⁷, —SO₂NR⁷¹R⁸¹ or —CONR⁷¹R⁸¹ substituents;
G¹ is hydrogen, C₀₋₁₀alkyl, C₂₋₁₀alkenyl, C₂₋₁₀alkynyl, —OR⁷², —SR⁷², —NR⁷²R⁸² (R⁹)ₙ₅, or G¹ and R³ taken together with the carbon atom to which they are attached form a 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
B is aryl or heteroaryl which is optionally substituted with one or more independent halo, —OR⁷⁷—SR⁷⁷, —NR⁷⁷R⁸⁸(R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthio C₂₋₁₀alkenyl, C₀₋₁₀alkylthio C₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with one or more independent halo, cyano, hydroxy, nitro, R⁶⁸, C₁₋₁₀alkoxy, —COOR⁷⁴, —(C═O)N(R⁷⁴)—, —NR⁷⁴CONR⁷⁴R⁷⁵, —NR⁷⁴COOR⁷⁵, —SO₂N^{R6}R⁸⁶, —NR⁷⁴SO₂NR⁷⁴R⁷⁵, or —NR⁷⁶R⁸⁶ substituents;
R^{4a}, R^{4b}, R^{4c}, R^{5a}, R^{5b} and R^{5c} are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthio C₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono(C₁₋₆alkyl)amino C₁₋₆alkyl, di(C₁₋₆alkyl)aminoC₁₋₆alkyl, mono(aryl)amino C₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or R^{4a} with R^{5a}, or R^{4b} with R^{5b}, or R^{4c} with R^{5C}, taken together with the respective carbon atom to which they are attached, form a carbonyl or 3-10 membered saturated or unsaturated monocyclic or polycyclic ring, wherein said ring is optionally substituted with R⁶⁹; or R^{4a} with R^{5a}, or R^{4b} with R^{5b}, or R^{4c} with R^{5c}, taken together with the respective carbon atom to which they are attached, form a 3-10 membered saturated or unsaturated monoheterocyclic or polyheterocyclic ring, wherein said ring is optionally substituted with R⁶⁹;
R^{6a}, R⁶⁶, R⁶⁷, R⁶⁸, and R⁶⁹ are each independently hydrogen, halo, —OR⁷⁷, —SH, —NR⁷⁷R⁸⁸(R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, C₁₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthio C₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)amino C₁₋₆alkyl, di(C₁₋₆alkyl)amino C₁₋₆alkyl, mono(aryl)amino C₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered mono or polycyclic saturated ring, mono or polycyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R⁸⁵, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynmyl, C₁₋₁₀alkylthioC₁₋₁₀alkyl, C₁₋₁₀alkylthio C₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl) or —N(C₀₋₄alkyl) (C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl) (C₀₋₄alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₄alkyl), —SO₂N(C₀₋₄ alkyl)(C₀₋₄alkyl) or —N(C₀₋₄alkyl) (C₀₋₄alkyl) substituents; and
n2, n3, n5, and n7 are each independently equal to 0, 1 or 2.

In another embodiment, a compound is represented by Formula (I), or an E or Z isomer thereof, syn or anti isomer thereof, an optically pure isomer thereof, or pharmaceutically acceptable salt thereof, wherein Y is as described below and the other variables are as described above.

In another embodiment, CYP26 inhibitors comprising the synergistic composition are represented by Formula (VIII):

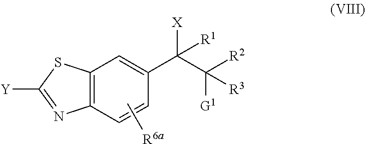

or an E or Z isomer thereof, syn or anti isomer thereof, an optically pure isomer thereof, or pharmaceutically acceptable salt thereof, wherein:
X is optionally substituted imidazolyl, triazolyl, benzimidazolyl, benzotriazolyl, 3-pyridinyl or 4-pyridinyl;
R¹ is hydrogen, C₀₋₆alkyl, —OR⁷, —SR⁷, or —NR⁷R⁸;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenyl carbonyl, C₂₋₁₀alkynylcarbonyl, C₁₋₁₀alkoxycarbonyl, C₁₋₁₀alkoxycarbonylC₁₋₁₀alkyl, mono C₁₋₆alkylaminocarbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR⁷⁷, —SO₂NR⁷¹R⁸¹ or —CONR⁷¹R⁸¹ substituents;
Y is hydrogen, halogen, —OR⁷⁴, —SR⁷⁴, —R⁷⁴, —C(═NR⁷⁴)NR⁷⁵R⁷⁶, —CN, —C(═NR⁷⁴)OR⁷⁵—, —COR⁷⁴, —R⁷⁴COR⁷⁵, —CR⁷⁴R⁷⁵COR⁷⁶, —COOR⁷⁴, —R⁷⁴COOR⁷⁵, —CR⁷⁴R⁷⁵COOR⁷⁶, —CONR⁷⁴R⁷⁵, —CR⁷⁴R⁷⁵CONR⁷⁴R⁷⁵, —NR⁷⁴COOR⁷⁵, —NR⁷⁴COR⁷⁵, —NR⁷⁴CONR⁷⁵R⁷⁶, —SO₂CR⁷⁴R⁷⁵, —SO₂NR⁷⁴R⁷⁵, —NR⁷⁴SO₂NR⁷⁵R⁷⁶, —NR⁷⁴R⁷⁵, aryl or heteroaryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents;
G¹ is hydrogen, C₀₋₁₀alkyl, C₂₋₁₀alkenyl, C₂₋₁₀alkynyl, —OR⁷², —SR⁷², —NR⁷²R⁸²(R⁹)ₙ₅, or G¹ and R³ taken together with the carbon atom to which they are attached form a 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸² (R⁹)ₙ₅, R⁷² and R⁸² taken together with the nitrogen atom to which they are attached form a 3-10 membered heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
R^{6a}, R⁶⁶, R⁶⁷, R⁶⁸, and R⁶⁹ are each independently hydrogen, halo, —OR⁷⁷, —SH, —NR⁷⁷R⁸⁸(R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, C₁₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)amino C₁₋₆alkyl, di(C₁₋₆alkyl)aminoC₁₋₆alkyl, mono(aryl)amino C₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered mono or polycyclic saturated ring, mono or polycyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R⁸⁵, R⁸⁶, R⁸⁷, R⁸⁸, R⁸⁸⁸, R⁹, R⁹⁵, and R⁹⁸ are each independently hydrogen, C₁₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynmyl, C₁₋₁₀alkylthioC₁₋₁₀alkyl, C₁₋₁₀alkylthio C₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl) or —N(C₀₋₄alkyl) (C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl) (C₀₋₄alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CON(C₀₋₄ alkyl)(C₀₋₄alkyl), —SO₂N(C₀₋₄ alkyl)(C₀₋₄alkyl) or —N(C₀₋₄alkyl) (C₀₋₄alkyl) substituents; and
n5 and n7 are each independently equal to 0, 1 or 2.

Furthermore, the compounds of the present invention include compounds represented by Formula (VIII) above, or a pharmaceutically acceptable salt thereof, and

1) wherein X is hetaryl, imidazolyl, triazolyl, benzimidazolyl, benzotriazolyl, 3-pyridinyl or 4-pyridinyl, any of which is optionally substituted with one or more independent R⁶⁶ substituents; or

2) wherein X is imidazolyl or triazolyl; or

3) wherein X is hetaryl, imidazolyl, or triazolyl, any of which is optionally substituted with one or more independent R⁶⁶ substituents, and Q¹ is —CO₂H or —CO₂R⁷⁵; or

4) wherein Y is nitrogen; or

5) wherein Y is nitrogen; and X is hetaryl, imidazolyl, triazolyl, 3-pyridinyl or 4-pyridinyl, any of which is optionally substituted with one or more independent R⁶⁶ substituents; or

6) wherein Y is nitrogen; and X is imidazolyl, or triazolyl; or

7) wherein Y is nitrogen; and X is imidazolyl, or triazolyl; n2 is 0; and Q¹ is —CO₂H or —CO₂R⁷⁵; or

8) wherein Y is nitrogen; and X is imidazolyl, or triazolyl; n3 is 0; and Q¹ is —CO₂H or —CO₂R⁷⁵; or

9) wherein Y is nitrogen; and X is imidazolyl, or triazolyl; n1 and n3 are 0;
and Q¹ is —CO₂H or —CO₂R⁷⁵; or

10) wherein Y is —NHCO—; or

11) wherein Y is —NHCO—; and X is hetaryl, imidazolyl, triazolyl, 3-pyridinyl or 4-pyridinyl, any of which is optionally substituted with one or more independent R⁶⁶ substituents; or

12) wherein Y is —NHCO—; and X is imidazolyl, or triazolyl; or

13) wherein Y is —NHCO—; and X is imidazolyl, or triazolyl; n2 and n3 are 0; and Q¹ is —CO₂H or —CO₂R⁷⁵; or

14) wherein Y is —NHCO—; and X is imidazolyl, or triazolyl; n3, n2 and n4 are 0; or

15) wherein Y is —NHCO—; and X is imidazolyl, or triazolyl; n2 is 0; and Q¹ is —CO₂H or —CO₂R⁷⁵; or

16) wherein Y is —NHCO—; and X is imidazolyl, or triazolyl; n3 is 0; and Q¹ is —CO₂H or —CO₂R⁷⁵; or

17) wherein Y is oxygen; or

18) wherein Y is oxygen; and X is hetaryl, imidazolyl, triazolyl, 3-pyridinyl or 4-pyridinyl, any of which is optionally substituted with one or more independent R⁶⁶ substituents; or

19) wherein Y is oxygen; and X is imidazolyl, or triazolyl; or

20) wherein Y is oxygen; and X is imidazolyl, or triazolyl; n1 and n3 are 0; and Q¹ is —CO₂H or —CO₂R⁷⁵; or

21) wherein Y is optionally substituted aryl or heteroaryl; or

22) wherein Y is optionally substituted phenyl; or

23) wherein Y is optionally substituted phenyl and X is hetaryl, imidazolyl, triazolyl, benzimidazolyl, benzotriazolyl 3-pyridinyl or 4-pyridinyl, any of which is optionally substituted with one or more independent R⁶⁶ substituents; or

24) wherein Y is optionally substituted phenyl and X is imidazolyl or triazolyl; or

25) wherein Y is phenyl substituted with halogen or CF₃; and X is imidazolyl or triazolyl; or

26) wherein Y is phenyl substituted with halogen or CF₃; and X is imidazolyl or triazolyl; n1, n2 and n3 are 0; and Q¹ is —CO₂H or —CO₂R⁷⁵; or

27) wherein Y is phenyl substituted with halogen or CF₃; and X is imidazolyl or triazolyl; n1, n2 and n3 are 0; G¹ is hydrogen; and Q¹ is —CO₂H or —CO₂R⁷⁵; or

28) wherein Y is phenyl and X is imidazolyl or triazolyl; n1, n2 and n3 are 0; and Q¹ is —CO₂H or —CO₂R⁷⁵; or

29) wherein Y is phenyl and X is imidazolyl or triazolyl; n1, n2 and n3 are 0; G¹ is hydrogen; and Q¹ is —CO₂H or —CO₂R⁷⁵; or

30) wherein Y is phenyl and X is imidazolyl or triazolyl; n2 and n3 are 0; and Q¹ is —CO₂H or —CO₂R⁷⁵; or

31) wherein Y is phenyl and X is imidazolyl or triazolyl; n2 and n3 are 0; G¹ is hydrogen; and Q¹ is —CO₂H or —CO₂R⁷⁵; or

32) wherein Y is phenyl and X is 3-pyridinyl or 4-pyridinyl; n2 and n3 are 0; G¹ is hydrogen; and Q¹ is —CO₂H or —CO₂R⁷⁵; or

33) wherein Y is phenyl and X is 3-pyridinyl or 4-pyridinyl; n2 and n3 are 0; G¹ is hydrogen; G¹ is hydrogen; and Q¹ is —CO₂H or —CO₂R⁷⁵; or

34) wherein Y is phenyl and X is imidazolyl or triazolyl; n1 and n2 are 0; and Q¹ is —CO₂H or —CO₂R⁷⁵; or

35) wherein Y is phenyl and X is imidazolyl or triazolyl; n1 and n2 are 0; G¹ is hydrogen; and Q¹ is —CO₂H or —CO₂R⁷⁵; or

36) wherein Y is phenyl and X is imidazolyl or triazolyl; n2 and n3 are 0; and Q¹ is tetrazole; or

37) wherein Y is phenyl and X is imidazolyl or triazolyl; n1 and n2 are 0; and Q¹ is tetrazole; or

38) where in Y is hydrogen; or

39) where in Y is a halogen; or

40) where in Y is chlorine or bromine; or

41) where in Y is R⁷⁴; or

42) where in Y is OR⁷⁴; or

43) where in Y is SR⁷⁴; or

44) where in Y is NH₂; or

45) where in Y is CONH₂; or

46) where in Y is COOR⁷⁶; or

47) where in Y is COOH; or

48) where in Y is COR⁷⁶; or

49) where in Y is COCF₃; or

50) where in Y is NHCOR⁷⁶; or

51) where in Y is NR⁷⁵R⁷⁶CONR⁷⁵R⁷⁶; or

52) where in Y is NHCONH2; or

53) where in Y is CN; or

54) where in Y is —C(═NR⁷⁴)NR⁷⁵R⁷⁶; or,

55) where in Y is NR⁷⁵R⁷⁶SONR⁷⁵R⁷⁶; or

56) where in Y is NR⁷⁵R⁷⁶SO₂NR⁷⁵R⁷⁶; or

57) where in Y is SO₂NR⁷⁵R⁷⁶; or

58) wherein X is hetaryl, imidazolyl, or triazolyl, any of which is optionally substituted with one or more independent R⁶⁶ substituents; R¹, R² and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is NH; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ7R⁷⁸, —SO₂NR⁷⁸R⁸⁸, or C₀₋₁₀alkyl; and Q¹ is —CO₂CH₃ or —CO₂iPr; or

59) wherein X is hetaryl, imidazolyl, or triazolyl, any of which is optionally substituted with one or more independent R⁶⁶ substituents; R¹, R² and R³ are each independently C₀₋₁₀alkyl; Y is NH or NHCO; Q¹ is C₀₋₆alkyl, CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷—SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸ (R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and G¹ is di(C₁₋₆alkyl)amino; or

60) wherein X is hetaryl, imidazolyl, or triazolyl, any of which is optionally substituted with one or more independent R⁶⁶ substituents; R¹, R² and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is NH or NHCO; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ7R⁷⁸, —SO₂NR⁷⁸R⁸⁸, or C₀₋₁₀alkyl; and R^{4b} and R^{5b} are both ethyl or are both methyl or are independently ethyl or methyl; or

61) wherein X is hetaryl, imidazolyl, or triazolyl, any of which is optionally substituted with one or more independent R⁶⁶ substituents; R¹, R² and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is NH or NHCO; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ7R⁷⁸, —SO₂NR⁷⁸R⁸⁸, or C₀₋₁₀alkyl; and Q¹ is —CO₂H; or

62) wherein X is hetaryl, imidazolyl, or triazolyl, any of which is optionally substituted with one or more independent R⁶⁶ substituents; R¹, R² and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is NHCO; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ7R⁷⁸, —SO₂NR⁷⁸R⁸⁸, or C₀₋₁₀alkyl; and R^{4a} with R^{5a} taken together with the respective carbon atom to which they are attached form a cyclopropyl, cyclobutyl, cyclopentyl, or cyclohexyl ring; or

63) wherein X is hetaryl, imidazolyl, or triazolyl, any of which is optionally substituted with one or more independent R⁶⁶ substituents; R¹, R² and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is oxygen; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₁₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ7R⁷⁸, —SO₂NR⁷⁸R⁸⁸, or C₀₋₁₀alkyl; and Q¹ is —CO₂H; or

64) wherein X is hetaryl, imidazolyl, or triazolyl, any of which is optionally substituted with one or more independent R⁶⁶ substituents; R¹, R² and R³ are each independently C₁₋₁₀alkyl; Y is oxygen; Q¹ is C₀₋₆alkyl, CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷—SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸ (R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and G¹ is di(C₁₋₆alkyl)amino; or

65) wherein X is hetaryl, imidazolyl, triazolyl, 3-pyridinyl or 4-pyridinyl, any of which is optionally substituted with one or more independent R⁶⁶ substituents; R¹, R² and R³ are each independently hydrogen or C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is optionally substituted phenyl; Q¹ is C₀₋₆alkyl, —CO₂R⁷⁵, or —CONR⁷⁴R⁸⁵; R^{4a}, R^{4b}, R^{5a}; and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a} or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, CONR⁷⁸R⁸⁸, —NO₂, —CN, —S(O)ₙ₇R⁷⁸, —SO₂NR⁷⁸R⁸⁸, or C₀₋₁₀alkyl; or

66) wherein X is imidazolyl or triazolyl; R¹ is hydrogen, R² and R³ are each independently C₁₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is optionally substituted phenyl; Q¹ is —_{C}O₂R⁷⁵ or —CONR⁷⁵R⁸⁵; R^{4a}; R^{4b}; R^{5a} and R^{5b} are each independently a C₁₋₁₀alkyl, any of which is optionally substituted with one or more independent halo, cyano, nitro, —OR⁷⁷, —SO₂ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each hydrogen; or

67) wherein X is hetaryl, imidazolyl, or triazolyl, any of which is optionally substituted with one or more independent R⁶⁶ substituents; R¹, R² and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is optionally substituted phenyl; Q¹ is C₀₋₆alkyl, —CO₂R or —CONR⁷⁵R⁸⁵; R^{4a} and R^{5a} are each hydrogen; or R^{4b} and R^{5b} are each independently a C₀₋₁₀alkyl, any of which is optionally substituted with R⁶⁹; or R^{4b} with R^{5b} taken together with the respective carbon atom to which they are attached form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; or

68) wherein X is hetaryl, imidazolyl, or triazolyl, any of which is optionally substituted with one or more independent R⁶⁶ substituents; R¹, R² and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is optionally substituted phenyl; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ7R⁷⁸, —SO₂NR⁷⁸R⁸⁸, or C₀₋₁₀alkyl; or

69) wherein X is hetaryl, imidazolyl, or triazolyl, any of which is optionally substituted with one or more independent R⁶⁶ substituents; R¹, R² and R³ are each independently C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is optionally substituted phenyl; Q¹ is C₀₋₆alkyl, —CO₂R or —CONR⁷⁵R⁸⁵; R^{4b} and R^{5b} are each independently C₀₋₆alkyl, or R^{4b} with R^{5b} taken together with the respective carbon atom to which they are attached form a 3-10 membered saturated ring; R^{4a} and R^{5a} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R⁵, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO², —CN, —S(O)ₙ7R⁷⁸, —SO₂NR⁷⁸R⁸⁸, or C₀₋₁₀alkyl; and Q¹ is —CO₂H; or

70) wherein X is hetaryl, imidazolyl, or triazolyl, any of which is optionally substituted with one or more independent R⁶⁶ substituents; R¹, R² and R³ are each independently C₀₋₁₀alkyl; Y is optionally substituted phenyl; Q¹ is C₀₋₆alkyl, CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷—SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸ (R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and G¹ is di(C₁₋₆alkyl)amino; or

71) wherein X is hetaryl, imidazolyl, or triazolyl, any of which is optionally substituted with one or more independent R⁶⁶ substituents; R¹, R² and R³ are each independently C₁₋₁₀alkyl; Y is optionally substituted phenyl; Q¹ is C₀₋₆alkyl, CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷—SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸ (R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, or C₀₋₁₀alkyl; and G¹ is di(C₁₋₆alkyl)amino, dimethylamino, ethylmethylamino, diethylamino, or isopropylmethylamino; or

72) wherein X is hetaryl, imidazolyl, or triazolyl, any of which is optionally substituted with one or more independent R⁶⁶ substituents; R¹, R² and R³ are each independently C₁₋₁₀alkyl; Y is optionally substituted phenyl; Q¹ is C₀₋₆alkyl, CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷—SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸ (R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and G¹ is heterocycle, pyrrolidine; or

73) wherein X is hetaryl, imidazolyl, or triazolyl, any of which is optionally substituted with one or more independent R⁶⁶ substituents; R¹ is C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is optionally substituted phenyl; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO², _{—C} N, —S(O)ₙ₇R⁷⁸, —SO₂NR⁷⁸R⁸⁸, or C₁₋₁₀alkyl; and R² and R³ are each independently hydrogen, methyl, or ethyl; or

74) wherein X is hetaryl, imidazolyl, or triazolyl, any of which is optionally substituted with one or more independent R⁶⁶ substituents; R¹ is C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is optionally substituted phenyl; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; R² is hydrogen; and G¹ and R³ taken together with the carbon atom to which they are attached form

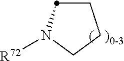

wherein is the carbon to which they are attached;
or G¹ and R³ taken together with the carbon atom to which they are attached form

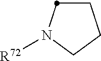

wherein is the carbon to which they are attached, any of which is optionally substituted by 1-10 independent R⁶⁷ substituents; or

75) wherein X is imidazole; or

76) wherein X is hetaryl, imidazolyl, or triazolyl, any of which is optionally substituted with one or more independent R⁶⁶ substituents; R¹ is C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is optionally substituted phenyl; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, or C₀₋₁₀alkyl; and R² is hydrogen and R³ is methyl; or

77) wherein X is hetaryl, imidazolyl, or triazolyl, any of which is optionally substituted with one or more independent R⁶⁶ substituents; R¹ is C₀₋₁₀alkyl; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is optionally substituted phenyl; Q¹ is C₀₋₆alkyl, —CO₂R⁷⁵, or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, or C₀₋₁₀alkyl; and R² is hydrogen and R³ is ethyl; or

78) wherein X is hetaryl, imidazolyl, triazolyl, 3-pyridinyl or 4-pyridinyl, any of which is optionally substituted with one or more independent R⁶⁶ substituents; R¹ is C₀₋₁₀alkyl; G¹ is hydrogen or —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶³ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is optionally substituted phenyl; Q¹ is C₀₋₆alkyl, —CO₂R⁷⁵, or —CONR⁷⁵R⁸⁵; R^{4a}; R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} and R^{6b} are each independently halo, —OR⁷⁸, —NR⁷⁸R⁸⁸(R⁹⁸)ₙ₇, —CO₂R⁷⁸, —CONR⁷⁸R⁸⁸, —NO², —CN, —S(O)ₙ₇R⁷⁸, —SO₂NR⁷⁸R⁸⁸, or C₀₋₁₀alkyl; and R² and R³ are independently methyl, ethyl or isopropyl; or

79) wherein X is imidazolyl or triazolyl; R¹ is hydrogen; G¹ is —NR⁷²R⁸² or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is optionally substituted phenyl; Q¹ is —CO₂R⁷⁵ or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} is hydrogen; R² is hydrogen; and R³ is methyl; or

80) wherein X is imidazolyl, triazolyl, 3-pyridinyl or 4-pyridinyl; R¹ is hydrogen; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is optionally substituted phenyl; Q¹ is —CO₂R⁷⁵ or —CONR⁷⁵R⁸⁵; R^{4a}; R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} is hydrogen; R² is hydrogen; and R³ is ethyl; or

81) wherein X is imidazolyl, triazolyl, 3-pyridinyl or 4-pyridinyl; R¹ is hydrogen; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is hydrogen, halogen, —OR⁷⁴, —SR⁷⁴, —R⁷⁴, —C(═NR⁷⁴)NR⁷⁵R⁷⁶, —CN, —C(═NR⁷⁴)OR⁷⁵—, —COR⁷⁴, —R⁷⁴COR⁷⁵, —CR⁷⁴R⁷⁵COR⁷⁶, —COOR⁷⁴, —R⁷⁴COOR⁷⁵, —CR⁷⁴R⁷⁵COOR⁷⁶, —CONR⁷⁴R⁷⁵, —CR⁷⁴R⁷⁵CONR⁷⁴R⁷⁵, —NR⁷⁴COOR⁷⁵, —NR⁷⁴COR⁷⁵, —NR⁷⁴CONR⁷⁵R⁷⁶, —SO₂CR⁷⁴R⁷⁵, —SO₂NR⁷⁴R⁷⁵, —NR⁷⁴SO₂NR⁷⁵R⁷⁶, —NR⁷⁴R⁷⁵, aryl or heteroaryl; and R^{6a} is hydrogen; and R² and R³ are independently methyl, ethyl or isopropyl; G¹ is heterocycle, pyrrolidine; or

82) wherein X is imidazolyl, triazolyl, 3-pyridinyl or 4-pyridinyl; R¹ is hydrogen; G¹ is hydrogen; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is hydrogen, halogen, —OR⁷⁴, —SR⁷⁴, —R⁷⁴, —C(═NR⁷⁴)NR⁷⁵R⁷⁶, —CN, —C(═NR⁷⁴)OR⁷⁵—, —COR⁷⁴, —R⁷⁴COR⁷⁵, —CR⁷⁴R⁷⁵COR⁷⁶, —COOR⁷⁴, —R⁷⁴COOR⁷⁵, —CR⁷⁴R⁷⁵COOR⁷⁶, —CONR⁷⁴R⁷⁵, —CR⁷⁴R⁷⁵CONR⁷⁴R⁷⁵, —NR⁷⁴COOR⁷⁵, —NR⁷⁴COR⁷⁵, —NR⁷⁴CONR⁷⁵R⁷⁶, —SO₂CR⁷⁴R⁷⁵, —SO₂NR⁷⁴R⁷⁵, —NR⁷⁴SO₂NR⁷⁵R⁷⁶, —NR⁷⁴R⁷⁵, aryl or heteroaryl; and R^{6a} is hydrogen; and R² and R³ are independently methyl, ethyl or isopropyl; or

83) wherein X is imidazolyl, triazolyl, 3-pyridinyl or 4-pyridinyl; R¹ is hydrogen; G¹ is hydrogen; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is optionally substituted phenyl; Q¹ is —CO₂R⁷⁵ or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} is hydrogen; and R² and R³ are independently methyl, ethyl or isopropyl; or

84) wherein X is imidazolyl or triazolyl; R′ is hydrogen; G¹ is —NR⁷²R⁸²; or G¹ and R³ taken together with the carbon atom to which they are attached form a 3-10 membered saturated ring, unsaturated ring, heterocyclic saturated ring, or heterocyclic unsaturated ring, wherein said ring is optionally substituted with one or more independent R⁶⁷ and an N heteroatom of the heterocyclic saturated ring or heterocyclic unsaturated ring optionally is substituted with an R⁷² substituent; or R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents; Y is optionally substituted phenyl; Q¹ is —CO₂R⁷⁵ or —CONR⁷⁵R⁸⁵; R^{4a}, R^{4b}, R^{5a}, and R^{5b} are each independently a C₀₋₁₀alkyl, any of which is optionally substituted with one or more independent halo, cyano, nitro, —OR⁷⁷, —SO₂NR⁷⁷R⁸⁷ or —NR⁷⁷R⁸⁷ substituents; or R^{4a} with R^{5a}, or R^{4b} with R^{5b} taken together with the respective carbon atom to which they are attached, form a 3-10 membered saturated or unsaturated ring, wherein said ring is optionally substituted with R⁶⁹; or R^{4a} with R^{5a}, or R^{4b} with R^{5b} taken together with the respective carbon atom to which they are attached form a 3-10 membered saturated or unsaturated heterocyclic ring, wherein said ring is optionally substituted with R⁶⁹; and R^{6a} is hydrogen; and R² and R³ are independently methyl, ethyl or isopropyl; and wherein, in each case, the other variables are as defined above for Formula (VIII).

In another embodiment, the CYP26 inhibitor is a compound of formula (IX):

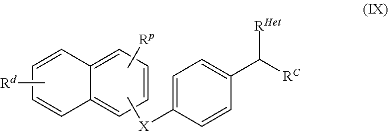

or an isomer, salt, solvate, chemically protected form or prodrug thereof,
wherein X is selected from O, S, NH or CH₂;
R^{d} and R^{P} are optional naphthyl group substituents;
R^{Het} is imidazolyl, triazolyl or pyridyl; and
R^{c} is C₁₋₄alkyl substituted by a group selected from: hydroxy, amino, amido, carboxy, C₁₋₇alkyl ester, C₅₋₇ aryl-C₁₋₂ alkyl ester, sulfonamino, sulfinamino, hydroxamino and tetrazolyl.
The compound of formula (IX) can be formula (IX-A) or formula (IX-B):

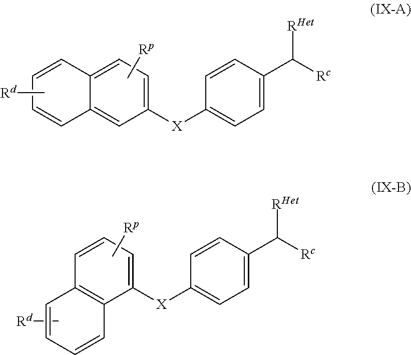

or an isomer, salt, solvate, chemically protected form or prodrug thereof,
wherein X is selected from O, S, NH or CH₂;
R^{Het} is imidazolyl, triazolyl or pyridyl; and
R^{c} is C₁₋₄alkyl substituted by a group selected from: hydroxy, amino, amido, carboxy, C₁₋₇alkyl ester, C₅₋₇ aryl-C₁₋₂ alkyl ester, sulfonamino, sulfinamino, hydroxamino and tetrazolyl; and
R^{d} and R^{P} are optionally selected from, but not limited to, C₁₋₇alkyl, C₃₋₂₀ heterocyclyl, C₅₋₂₀ aryl, halo, hydroxy, ether, nitro, cyano, acyl, ester, amido, amino, acylamido, ureido, acyloxy, thiol, thioether, sulfoxide, sulfonyl, thioamido, sulfonamino, sulfinamino and hydroxamino.

Particular compounds of interest in formula (IX) include 3-imidazol-1-yl-2,2-dimethyl-3-[4-(naphthalen-2-ylamino)-phenyl]-propionic acid methyl ester, methyl anti-3-(1H-1-imidazolyl)-3-[4-(2-naphthylamino)phenyl]-2-methylpropanoate; methyl syn-3-(1H-1-imidazolyl)-3-[4-(2-naphthylamino)phenyl]-2-methylpropanoate; methyl 3-(1H-imidazol-1-yl)-2,3-dimethyl-3-((4-naphthalene-2-yloxy)phenyl)propanoate; methyl 2,2-dimethyl-3-(4-(naphthalen-2-ylamino)phenyl)-3-(1H-1,2,4-triazol-1-yl)propanoate, 3-imidazol-1-yl-2,2-dimethyl-3-[4-(naphthalen-2-ylamino)-phenyl]-propionic acid; 3-imidazol-1-yl-2,2-dimethyl-3-[4-(naphthalen-2-ylamino)-phenyl]-propionic acid ethyl ester; 3-imidazol-1-yl-2,2-dimethyl-3-[4-(naphthalen-2-ylamino)-phenyl]-propionic acid isopropyl ester; 3-imidazol-1-yl-2,2-dimethyl-3-[4-(naphthalen-2-ylamino)-phenyl]-propionic acid butyl ester; 3-imidazol-1-yi-N-isopropyl-2,2-dimethyl-3-[4-(naphthalen-2-ylamino)-phenyl]-propionamide; 2,2-dimethyl-3-(methyl-vinyl-amino)-3-[4-(naphthalen-2-ylamino)-phenyl]-propionamide; 3-imidazol-1-yl-2,2, N-trimethyl-3-[4-(naphthalen-2-ylamino)-phenyl]-propionamide; 3-imidazol-1-yl-2,2, N,N-tetramethyl-3-[4-(naphthalen-2-ylamino)-phenyl]-propionamide; 3-imidazol-1-yl-3-[4-(6-methoxy-naphthalen-2-ylamino)-phenyl]-2,2-dimethyl-propionic acid methyl ester; 3-[4-(6-hydroxy-naphthalen-2-ylamino)-phenyl]-3-imidazol-1-yl-2,2-dimethyl-propionic acid methyl ester; 3-imidazol-1-yl-3-[4-(6-methoxy-naphthalen-2-ylamino)-phenyl)-2,2-dimethyl-propan-1-ol; 3-imidazol-1-yl-2,2-dimethyl-3-[4-(naphthalen-1-ylamino)-phenyl)-propionic acid methyl ester; 3-[4-(2-ethoxy-naphthalen-1-ylamino)-phenyl]-3-imidazol-1-yl-2,2-dimethyl-propionic acid methyl ester; 3-imidazol-1-yl-2,2-dimethyl-3-[4-(naphthalen-2-ylamino)-phenyl)-propionic acid benzyl ester; and 3-imidazol-1-yl-2,2-dimethyl-3-[4-(naphthalen-2-ylamino)-phenyl)-propionic acid pentyl ester.

It will be appreciated that each of the CYP26 inhibitor compounds described herein and each of the classes and subclasses of compounds described above (I-IX) may be substituted as described generally herein, or may be substituted according to any one or more of the subclasses described above and herein.

Some of the foregoing compounds can comprise one or more asymmetric centers, and thus can exist in various isomeric forms, e.g., stereoisomers and/or diastereomers. Thus, inventive compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

Compounds comprising synergistic compositions of the invention may be prepared by crystallization of compound of formula (I) (IX) under different conditions and may exist as one or a combination of polymorphs of compound of general formula (I) (IX) forming part of this invention. For example, different polymorphs may be identified and/or prepared using different solvents, or different mixtures of solvents for recrystallization; by performing crystallizations at different temperatures; or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other techniques. Thus, the present invention encompasses inventive compounds, their derivatives, their tautomeric and geometrical isomeric forms, their stereoisomers, their polymorphs, their pharmaceutically acceptable salts their pharmaceutically acceptable solvates and pharmaceutically acceptable compositions containing them. Tautomeric forms of compounds of the present invention include, pyrazoles, pyridones and enols, etc., and geometrical isomers include EZ isomers of compounds having double bonds and cis-trans isomers of monocyclic or fused ring systems, etc.

### 2) Pharmaceutical Compositions

In practice, the methods and synergistic compositions embodied herein comprise administering a PI3Ki, a retinoid and optionally a CYP26 inhibitor to a subject in need thereof to provide benefit to the subject more than is achievable by either inhibitor when administered in the absence of the other. Therefore, a synergistic composition can comprise both inhibitors in the same composition or separately for simultaneous administration, or in other embodiments, each inhibitor can be administered to the subject to achieve its maximal pharmacological activity, which in the presence of the pharmacological activity of the other inhibitor, provides a synergistic benefit. Compositions as described hereinbelow can comprise the PI3Ki and the retinoid, or the PI3Ki, retinoid and CYP26 inhibitor, and can refer to the components individually or the combination.

One or more of the PI3Ki compounds and the retinoid compounds described herein, or another as generally known to one of ordinary skill in the art, or the CYP26 inhibitor compounds represented by Formulas (I)-(IX) or another as generally known to one of skill in the art, or pharmaceutically acceptable salts thereof, in accordance with this invention can be combined as the active ingredient in intimate admixture with a pharmaceutical carrier according to conventional pharmaceutical compounding techniques. Or at least one of each of the two inhibitors can be combined together. The following discussion pertains to either inhibitor separately, or a combination if it provides the requisite pharmacology. The carrier may take a wide variety of forms depending on the pharmacokinetics of the compound and form of preparation desired for administration. e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the inhibitor compound, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredients with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of an inhibitor herein. Compounds or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of each active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by any one of Formula (I)-(IX) of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described herein, or pharmaceutically acceptable salts thereof, may also be prepared in powder or liquid concentrate form.

Generally, dosage levels on the order of from about 0.01 mg/kg to about 1 50 mg/kg of body weight per day are useful in the treatment of the above indicated conditions, or alternatively about 0.5 mg to about 7 g per patient per day. For example, dermatological diseases and cancers may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

As discussed above this invention provides novel compounds that have biological properties useful for the treatment of any of a number of conditions or diseases in which inhibition of PI3K, optionally CYP26 or the activities thereof have a therapeutically useful role and the effect is synergistic. Further description of pharmaceutical compositions is provided herein below.

Accordingly, in another aspect of the present invention, pharmaceutical compositions are provided, which comprise any one or more of the compounds described herein (or a prodrug, pharmaceutically acceptable salt or other pharmaceutically acceptable derivative thereof), and optionally comprise a pharmaceutically acceptable carrier. In certain embodiments, these compositions optionally further comprise one or more additional therapeutic agents. Alternatively, a compound of this invention may be administered to a patient in need thereof in combination with the administration of one or more other therapeutic agents. For example, additional therapeutic agents for conjoint administration or inclusion in a pharmaceutical composition with a compound of this invention may be an approved agent to treat the same or related indication, or it may be any one of a number of agents undergoing approval in the Food and Drug Administration that ultimately obtain approval for the treatment of any disorder related to fibrosis. It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood, or N-demethylation of a compound of the invention where R¹ is methyl. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference. By way of example, N-methylated pro-drugs of the compounds of the invention are embraced herein.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogenfree water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut (peanut), corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

In one embodiment, liquid compositions or liquid formulations comprising compounds of the invention are provided that have increased solubility as compared to compounds of the invention dissolved in aqueous buffer such as phosphate-buffered saline. In one embodiment, such liquid compositions with increased solubility are provided by a composition comprising polyethylene glycol, polysorbate or a combination thereof. In one embodiment, the polyethylene glycol is polyethylene glycol 300. In another embodiment the polysorbate is polysorbate 80. In another embodiment the polyethylene glycol is present at about 40% to about 60% (v/v). In another embodiment the polysorbate is present at about 5% to about 15% (v/v). In another embodiment the polyethylene glycol is present at about 50% (v/v). In another embodiment the polysorbate is present at about 10% (v/v). In one formulation, the polyethylene glycol is present at 50% (v/v) together with polysorbate 80 at 10% (v/v). The balance of the solution can be a saline solution, a buffer or a buffered saline solution, such as phosphate-buffered saline. The pH of the solution can be from about pH 5 to about pH 9, and in other embodiments, about from pH 6 to about pH 8. In one embodiment the pH of the buffer is 7.4. In the foregoing embodiments, the compound of the invention is soluble at a concentration higher than in buffer alone, and can be present at about 0.8 to about 10 milligrams per milliliter of solution, or even higher. These formulations offer the preparation of convenient dosing solutions of practical volumes for single dose administration, by any route, in particular a parenteral route. In one embodiment, the route is intravenous, subcutaneous or intraperitoneal. Such compositions with a higher solubility permit achievement of more elevated blood concentrations that provide efficacy when the threshold Cmax (maximal blood concentration after administration) should be achieved for optimal efficacy.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

In other embodiments, solid dosage forms of compounds embodied herein are provided. In some embodiment, such solid dosage forms have improved oral bioavailability. In one embodiment, a formulation is prepared in a solid formulation comprising about 20% (w/w) compound of the invention, about 10-20% (w/w) GLUCIRE® 4414, about 10-20% (w/w) vitamin E succinate (TPS), 0 to about 60% polyethylene glycol 400, 0 to about 40% Lubrizol, 0 to about 15% Cremophor RH 40 (w/w), and about 1% (w/w) BHT. Formulations containing Cremophor RH 20 are liquid at room temperature but waxy solids at 4 C. The foregoing examples of one or more agents to aid in preparing formulations of inventive compound are merely illustrative and non-limiting.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

In other embodiments solid dosage forms are provided. In certain embodiments, such solid dosage forms provide a higher than about a 20% oral bioavailability. As will be shown in the examples below, compounds of the invention can be co-precipitated with one or more agents such as mannitol, a combination of mannitol and lactobionic acid, a combination of mannitol and gluconic acid, a combination of mannitol and methanesulfonic acid, a combination of microcrystalline cellulose and oleic acid or a combination of pregelatinized starch and oleic acid. The foregoing examples of one or more agents to aid in preparing formulations of inventive compound are merely illustrative and non-limiting. Non-limiting examples of inventive compounds in such solid dosage forms include

The present invention encompasses pharmaceutically acceptable topical formulations of inventive compounds. The term “pharmaceutically acceptable topical formulation”, as used herein, means any formulation which is pharmaceutically acceptable for intradermal administration of a compound of the invention by application of the formulation to the epidermis. In certain embodiments of the invention, the topical formulation comprises a carrier system. Pharmaceutically effective carriers include, but are not limited to, solvents (e.g., alcohols, poly alcohols, water), creams, lotions, ointments, oils, plasters, liposomes, powders, emulsions, microemulsions, and buffered solutions (e.g., hypotonic or buffered saline) or any other carrier known in the art for topically administering pharmaceuticals. A more complete listing of art-known carriers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations of the invention may comprise excipients. Any pharmaceutically acceptable excipient known in the art may be used to prepare the inventive pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerine, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the invention include, but are not limited to, vitamin E oil, allatoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in the formulation, available manufacturing equipment, and costs constraints. As used herein the term “penetration enhancing agent” means an agent capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). In certain exemplary embodiments, penetration agents for use with the invention include, but are not limited to, triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate) and N-methyl pyrrolidone.

In certain embodiments, the compositions may be in the form of ointments, pastes, creams, lotions, gels, powders, solutions, sprays, inhalants or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this invention. Formulations for intraocular administration are also included. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anti-inflammatory agent), or they may achieve different effects (e.g., control of any adverse effects). In non-limiting examples, one or more compounds of the invention may be formulated with at least one cytokine, growth factor or other biological, such as an interferon, e.g., alpha interferon, or with at least another small molecule compound. Non-limiting examples of pharmaceutical agents that may be combined therapeutically with compounds of the invention include: antivirals and antifibrotics such as interferon alpha, combination of interferon alpha and ribavirin, Lamivudine, Adefovir dipivoxil and interferon gamma; anticoagulants such as heparin and warfarin; antiplatelets e.g., aspirin, ticlopidine and clopidogrel; other growth factors involved in regeneration, e.g., VEGF and FGF and mimetics of these growth factors; antiapoptotic agents; and motility and morphogenic agents.

In certain embodiments, the pharmaceutical compositions of the present invention further comprise one or more additional therapeutically active ingredients (e.g., anti-inflammatory and/or palliative). For purposes of the invention, the term “Palliative” refers to treatment that is focused on the relief of symptoms of a disease and/or side effects of a therapeutic regimen, but is not curative. For example, palliative treatment encompasses painkillers, antinausea medications and anti-sickness drugs.

Notwithstanding the above discussion on combinations, as noted herein, the retinoid and the CYP26 inhibitor can be administered in the same composition, or in other embodiments, separately, provided that the synergistic benefit to the patient of the two inhibitors is achieved. For example, one inhibitor may administered parenterally and the other orally. The frequency of administration may depend on the pharmacokinetics of each component such that the synergistic activity can be optimized without necessarily requiring simultaneous administration or administration by the same routes.

### Pharmaceutical Uses and Methods of Treatment

In certain embodiments, the method involves the administration of a therapeutically effective amount of the synergistic composition or a pharmaceutically acceptable derivative thereof to a subject (including, but not limited to a human or animal) in need of it. Subjects for which the benefits of the compounds of the invention are intended for administration include, in addition to humans, livestock, domesticated, zoo and companion animals.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, subcutaneously, intradermally, intra-ocularly, topically (as by powders, ointments, or drops), buccally, as an oral or nasal spray, or the like, depending on the severity of the disease or disorder being treated. In certain embodiments, each compound in the compositions of the invention may be administered at dosage levels of about 0.001 mg/kg to about 50 mg/kg, preferably from about 0.1 mg/kg to about 10 mg/kg for parenteral administration, or preferably from about 1 mg/kg to about 50 mg/kg, more preferably from about 10 mg/kg to about 50 mg/kg for oral administration, of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally. The foregoing ranges apply to each individual compound in the compositions embodied herein.

As noted above, methods for treating a subject using the combination of a PI3Ki, a retinoid and optionally a CYP26 inhibitor do not have to be administered simultaneously or by the same route of administration, but the treatment in one embodiment is beneficial synergistically over the effects of one agent administered without the other. By way of non-limiting examples, the PI3Ki can be administered orally, the retinoid can be administered topically and the optional CYP26 inhibitor orally; or the PI3Ki subcutaneously, the retinoid orally and optional the CYP26 inhibitor topically.

Moreover, pharmaceutical compositions comprising one or more compounds of the invention may also contain other compounds or agents for which co-administration with the compound(s) of the invention is therapeutically advantageous. As many pharmaceutical agents are used in the treatment of the diseases and disorders for which the compounds of the invention are also beneficial, any may be formulated together for administration. Synergistic formulations are also embraced herein, where the combination of at least one compound of the invention and at least one other compounds act more beneficially than when each is given alone. Non-limiting examples of pharmaceutical agents that may be combined therapeutically with compounds of the invention include (non-limiting examples of diseases or conditions treated with such combination are indicated in parentheses): antivirals and antifibrotics, such as interferon alpha (hepatitis B, and hepatitis C), combination of interferon alpha and ribavirin (hepatitis C), Lamivudine (hepatitis B), Adefovir dipivoxil (hepatitis B), interferon gamma (idiopathic pulmonary fibrosis, liver fibrosis, and fibrosis in other organs); anticoagulants, e.g., heparin and warfarin (ischemic stroke); antiplatelets e.g., aspirin, ticlopidine and clopidogrel (ischemic stroke); other growth factors involved in regeneration, e.g., VEGF and FGF and mimetics of these growth factors; antiapoptotic agents; and motility and morphogenic agents. All-trans retinoic acid and active analogs are also provided as combination therapy.

### Treatment Kit

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### Equivalents

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### EXEMPLIFICATION

The methods and compositions of this invention can be understood further by the examples that illustrate some of the processes by which these compounds are prepared or used. It will be appreciated, however, that these examples do not limit the invention. Variations of the invention, now known or further developed, are considered to fall within the scope of the present invention as described herein and as hereinafter claimed.

PI3K inhibitors useful in the methods and compositions herein include 2-(1H-indazol-4-yl)-6-(4-methanesulfonyl-piperazin-1-ylmethyl)-4-morpholin-4-yl-thieno[3,2-d]pyrimidine (GDC-0941), BEZ235, BKM120, PX866, BAY 80-6946, GDC-0032, GSK 2363771, IP-145 or CAL-101, as described herein above, as non-limiting examples.

A retinoid useful for the methods and compositions herein includes retinol ((2E,4E,6E,8E)-3,7-dimethyl-9-(2,6,6-trimethyl cyclohex-1-enyl)nona-2,4,6,8-tetraen-1-ol) or ATRA, by way of non-limiting example.

CYP26 inhibitors can be obtained following the guidance of PCTUS2011038695, published as WO2011153192, and in PCTUS201002175, published as WO2011016863; talarozole (RAMBAZOLE), having the chemical name N-(2-benzothioazolyl)-N-[4-[2-ethyl-1-(1,2,4-triazo-1-yl)butyl]phenyl]amine; 1-[benzofuran-2-yl-(4-alkyl/aryl-phenyl)-methyl]-1H-triazoles, methyl 3-(4-(aryl-2-ylamino)phenyl)propanoates 3-(1H-imidazol- and triazol-1-yl)-2,2-dimethyl-3-[4-(naphthalen-2-ylamino)phenyl]propyl derivatives. Other CYP26 inhibitors include (±)-4-(1H-imidazol-1-yl)-methyl retinoate, (±)-4-(1H-imidazole-1-yl)retinoic acid, (±)-4-(1H-1,2,4-triazol-1-yl) methyl retinoate, (±)-4-(4H-1,2,4-triazole-4-yl) methyl retinoate, (±)-4-(1H-1,2,4-triazol-1-yl) retinoic acid, and (±)-4-(4H-1,2,4-triazol-4-yl) retinoic acid.

### Example 1

### ATRA Synergizes with PI3Km/TOR Inhibitors in Breast Cancer

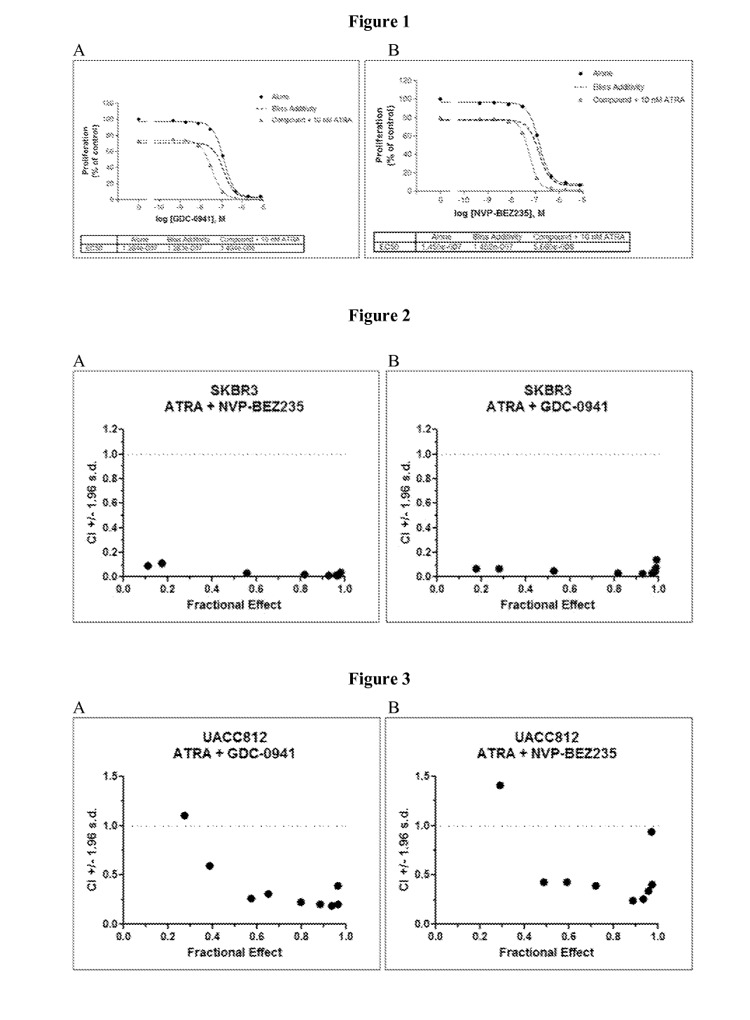

The effect of ATRA in combination with GDC-0941 or NVP-BEZ235 on the proliferation of SK-BR-3 breast cancer cells was evaluated. SK-BR-3 cells are sensitive to all trans retinoic acid (ATRA), with an estimated IC50 of 40 nM. To test the combination of ATRA and PI3Km/TOR inhibitors, cells were plated in a 96-well plate and treated with compounds in a “checkerboard” design. After 7 days the cell proliferation was determined using the Cell Titer Glo reagent (Promega). In the absence of ATRA, both GDC-0941 and NVP-BEZ235 inhibited the proliferation of SK-BR-3 growth, with an estimated IC50 of 126 nM for GDC-0941 and 145 nM for NVP-BEZ235 (FIG. 1A, B). However, in the presence of ATRA, both compounds were significantly more potent. At 10 nM ATRA, a concentration that by itself only mildly inhibited cell proliferation, GDC-0941 had an IC50 of 35 nM and NVP-BEZ235 had an IC50 of 57 nM. The bliss additivity model predicts the shape of the curve and magnitude of growth inhibition if the effects of two compounds is additive. The fact that combined effect is greater than expected for additivity shows that ATRA and the PI3Km/TOR inhibitors synergize to inhibit breast cancer proliferation.

Another method to determine the interaction between two compounds is the combination index (CI) method, which can be calculated by the computer program Calcusyn. Combination indices well below 0.2 were found, indicating strong synergy between the PI3Km/TOR inhibitors and ATRA (FIG. 2, A-B)

The combination of ATRA with GDC-0941 or NVP-BEZ235 against breast cancer cell line UACC812 was also evaluated. For these experiments, a fixed ratio design was used, where the ratio of PI3Km/TOR inhibitor to ATRA was 3:1. Also in this cell line and with this experimental protocol, CI's well below 1 were found, indicating synergy (FIG. 3, A-B). Taken together with the results in SK-BR-3 cells, these results in UACC812 cells show potential of synergy between retinoid-based therapies and other molecularly targeted therapies, such as PI3Km/TOR inhibitors.

What is claimed is:

1. A composition comprising a combination of a phosphatidylinositol 3-kinase inhibitor and a retinoid, wherein said combination is effective to suppresses fibrosis or the growth of dysproliferative cells in vivo.

2. The composition of claim 1 further comprising a CYP26 inhibitor.

3. The composition of claim 1 wherein the retinoid is retinol, 9-cis retinoic acid, 13-cis retinoic acid, all-trans retinoic acid, fenretinide, 6-[3-(1-adamantyl)-4-hydroxyphenyl]-2-naphthalene carboxylic acid or a retinyl ester.

4. The composition of claim 3 wherein the retinyl ester is retinyl acetate or retinyl palmitate.

5. The composition of claim 2 wherein the CYP26 inhibitor is a compound of formula (I) 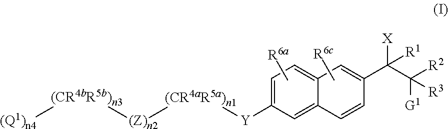
or an E or Z isomer thereof, syn or anti isomer thereof, an optically pure isomer thereof, or a pharmaceutically acceptable salt thereof, wherein:
X is an unsaturated heterocycle selected from pyrrolyl, pyrazolyl, imidazolyl, triazolyl, tetrazolyl, thiazole, or pyridinyl, any of which is optionally substituted with one or more independent R⁶⁶ substituents;
R¹ is a C₀₋₆alkyl, —OR⁷, —SR⁷, or —NR⁷R⁸;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkyl amino carbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR—SO₂NR⁷¹R⁸¹ or —CONR⁷¹R⁸¹ substituents;
G¹ is —OR⁷², —SR⁷², —NR⁷²R⁸²(R⁹)ₙ₅, or G¹ and R³ taken together with the carbon atom to which they are attached form a 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Y is an oxygen atom, sulfur atom, —(C═O)N(R⁷⁴)—, —CR^{4c}R^{5c} or —NR⁷⁴;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with R⁶⁸;
Q¹ is C₀₋₆alkyl, —OR⁷⁵, —NR⁷⁵R⁸⁵(R⁹⁵)ₙ₆, —CO₂R⁷⁵, —CONR⁷⁵R⁸⁵, —(C═S)OR⁷⁵, —(C═O)SR⁷⁵, —NO₂, —CN, halo, —S(O)ₙ₆R⁷⁵, —SO₂NR⁷⁵R⁸⁵, —NR⁷⁵(C═NR⁷⁷⁵)NR⁷⁷⁷⁵R⁸⁵, —NR⁷⁵(C═NR⁷⁷⁵)OR⁷⁷⁷⁵, —NR⁷⁵(C═NR⁷⁷⁵)SR⁷⁷⁷⁵, —O(C═O)OR⁷⁵, —O(C═O)NR⁷⁵R⁸⁵, —O(C═O)SR⁷⁵, —S(C═O)OR⁷⁵, —S(C═O)NR⁷⁵R⁸⁵, —S(C═O)SR⁷⁵, —NR⁷⁵(C═O)NR⁷⁷⁵R⁸⁵, or —NR⁷⁵(C═S)NR⁷⁷⁵R⁸⁵; in the case of —NR⁷⁵R⁸⁵ (R⁹⁵)ₙ₆, R⁷⁵ and R⁸⁵ taken together with the nitrogen atom to which they are attached form a 3-10 membered saturated ring, unsaturated ring, heterocyclic saturated ring, or heterocyclic unsaturated ring, any of which is optionally substituted with one or more independent halo, cyano, hydroxy, nitro, C1-10alkoxy, —SO₂N^{R6}R⁸⁶ or —NR⁷⁶R⁸⁶ substituents;
R^{4a}, R^{4b}, R^{4c}, R^{5a}, R^{5b}, and R^{5c} are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C1-10alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or hetaryl-C₁₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or R^{4a} with R^{5a}, or R^{4b} with R^{5b}, or R^{4c} with R^{5C}, taken together with the respective carbon atom to which they are attached, form a carbonyl or 3-10 membered saturated or unsaturated ring, wherein said ring is optionally substituted with R⁶⁹; or R^{4a} with R^{5a}, or R^{4b} with R^{5b}, or R^{4c} with R^{5c}, taken together with the respective carbon atom to which they are attached, form a 3-10 membered saturated or unsaturated heterocyclic ring, wherein said ring is optionally substituted with R⁶⁹;
R^{6a}, R^{6b}, R⁶⁶, R⁶⁷, R⁶⁸, and R⁶⁹ are each independently halo, —OR—SH, —NR⁷⁷R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₀₋₁₀alkylthio C₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenyl C₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂-loalkenyl, or hetaryl-C₂₋₁₀alkynyl, any of which is optionally substituted with one or more independent halo, cyano, nitro, —OR, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)aminoC₁₋₆alkyl, di(C₁₋₆alkyl)aminoC₁₋₆alkyl, mono(aryl)amino C₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R85, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C2-loalkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄ alkyl)(C₀₋₄alkyl) or —N(C₀₋₄ alkyl) (C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₁₀alkyl), —SO₂N(C₀₋₄alkyl)(C₀₋₄ alkyl) or —N(C₀₋₄ alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄ alkyl)(C₀₋₄alkyl) or —N(C₀₋₄ alkyl) (C₀₋₄ alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₄alkyl), —SO₂N(C₀₋₄ alkyl)(C₀₋₄alkyl) or —N(C₀₋₄ alkyl) (C₀₋₄ alkyl) substituents; and
n1, n2, n3, n4, n5, n6, and n7 are each independently equal to 0, 1 or 2.

6. The composition of claim 2 wherein the CYP26 inhibitor is a compound of formula (II) 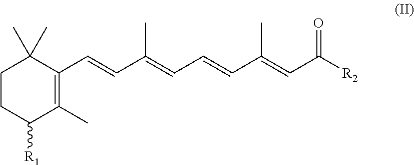
and E or Z isomers thereof, syn or anti isomers thereof, optically pure isomers thereof, or pharmaceutically acceptable salt thereof, and a pharmaceutically acceptable carrier therefor, wherein:
R₁ is an optionally substituted azole, sulfur, oxygen, nitrogen, pyridyl, acetylinic, cyclopropyl-amine, ester, oxime, cyano, amino, azido, cyclopropylamino, oxirane, aziridine, thiirane, thiol, alkylthiol, —OR₄ wherein R₄ is hydrogen or an alkyl group, cyclopropylether, an oxygen containing group that forms, together with the 4-position carbon, an oxirane group; —NR⁵R⁶, where R⁵ and R⁶ are independently selected from the group consisting of hydrogen and alkyl groups, or R⁵ and R⁶ may together form a ring;
R² is selected from the group consisting of hydroxyl, aminophenol, —OR₃ and an azole group, and
R³ is selected from the group consisting of alkyl, aryl and heterocyclic groups.

7. The composition of claim 2 wherein the CYP26 inhibitor is a compound of formula (III) 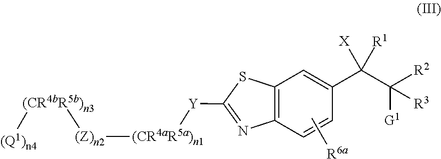
or an E or Z isomer thereof, syn or anti isomer thereof, an optically pure isomer thereof, or pharmaceutically acceptable salt thereof, wherein:
X is an unsaturated heterocycle selected from pyrrolyl, pyrazolyl, imidazolyl, triazolyl, benzimidazolyl, benzotriazolyl, tetrazolyl, thiazole, 3-pyridinyl or 4-pyridinyl, any of which is optionally substituted with one or more independent R⁶⁶ substituents;
R¹ is hydrogen, C₀₋₆alkyl, —OR⁷, —SR⁷, or —NR⁷R⁸;
R² and R³ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthio C₂₋₁₀alkenyl, C₁₋₁₀alkylthio C₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, mono C₁₋₆alkylaminocarbonyl, diC₁₋₆aminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂NR⁷¹R⁸¹, or —NR⁷¹R⁸¹ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷¹, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷¹R⁸¹, —SO₂NR⁷¹R⁸¹ or —NR⁷¹R⁸¹ substituents;
or R² and R³ taken together with the carbon atom to which they are attached form a 3-10 membered saturated ring, unsaturated ring, heterocyclic saturated ring, or heterocyclic unsaturated ring, wherein said ring is optionally substituted with one or more independent C₁₋₆alkyl, halo, cyano, nitro, —OR⁷⁷—SO₂NR⁷¹R⁸¹ or —CONR⁷¹R⁸¹ substituents;
G¹ is hydrogen, C₀₋₁₀alkyl, C₂₋₁₀alkenyl, C₂₋₁₀alkynyl, —OR⁷², —SR⁷², —NR⁷²R⁸²(R⁹)ₙ₅, or G¹ and R³ taken together with the carbon atom to which they are attached forma 3-10 membered saturated ring, unsaturated ring, heterocyclic saturated ring, or heterocyclic unsaturated ring, any of which is optionally substituted with one or more independent R and an N heteroatom of the heterocyclic saturated ring or heterocyclic unsaturated ring optionally is substituted with an R⁷² substituent; or in the case of —NR⁷²R⁸²(R⁹)ₙ₅, R⁷² and R⁸² taken together with the nitrogen atom to which they are attached form a 3-10 membered heterocyclic saturated ring, or heterocyclic unsaturated ring, wherein said ring is optionally substituted with one or more independent halo, cyano, hydroxy, nitro, C₁₋₁₀alkoxy, —SO₂NR⁷³R⁸³ or —NR⁷³R⁸³ substituents;
Y is a bond, hydrogen, halogen, an oxygen atom, sulfur atom, —CN, R⁷⁴, —C(═NR⁷⁴)NR⁷⁵R⁷⁶, —C(═NR⁷⁴)OR⁷⁵—, —COR⁷⁴, —R⁷⁴COR⁷⁵, —CR⁷⁴R⁷⁵COR⁷⁶, —COOR⁷⁴, —R⁷⁴COOR⁷⁵, —CR⁷⁴R⁷⁵COOR⁷⁶, —CONR⁷⁴R⁷⁵, —CR⁷⁴R⁷⁵CONR⁷⁴R⁷⁵, —NR⁷⁴COOR⁷⁵, —NR⁷⁴COR⁷⁵, NR⁷⁴CONR⁷⁵R⁷⁶, —SO₂CR⁷⁴R⁷⁵, —SO₂NR⁷⁴R⁷⁵, —NR⁷⁴SO₂NR⁷⁵R⁷⁶, —NR^{6b}CO—, —NR^{6b}R⁷⁵, aryl or heteroaryl, any of which is optionally substituted with one or more independent halo, cyano, hydroxy, nitro, R⁶⁸, —SO₂N^{R6}R⁸⁶ or —NR⁷⁶R⁸⁶ substituents;
Z is -aryl-, -arylalkyl-, -aryloxy-, -oxyaryl-, -arylalkenyl-, -alkenylaryl-, -hetaryl-, -hetarylalkyl-, -alkylhetaryl-, -hetarylalkenyl-, -alkenylhetaryl-, or -aryl-, any of which is optionally substituted with one or more independent halo, cyano, hydroxy, nitro, R⁶⁸, C₁₋₁₀alkoxy, —COOR⁷⁴, —(C═O)N(R⁷⁴)—, —NR⁷⁴CONR⁷⁴R⁷⁵, —NR⁷⁴COOR⁷⁵, —SO₂N^{R6}R⁸⁶, —NR⁷⁴SO₂NR⁷⁴R⁷⁵, or —NR⁷⁶R⁸⁶ substituents;
Q¹ is C₀₋₆alkyl, cycloC₃₋₈alkyl, bridged bicycloalkyl, —OR⁷⁵, —COR⁷⁴, —NR⁷⁵R⁸⁵(R⁹⁵)ₙ₆, —CO₂R⁷⁵, —CONR⁷⁵R⁸⁵, —(C═S)OR⁷⁵, —(C═O)SR⁷⁵, —NO₂, —CN, halo, —S(O)ₙ₆R⁷⁵, —SO₂NR⁷⁵R⁸⁵, —NR⁷⁵(C═NR⁷⁷⁵)NR⁷⁷⁷⁵R⁸⁵, —NR⁷⁵(C═NR⁷⁷⁵)OR⁷⁷⁷⁵, —NR⁷⁵(C═NR⁷⁷⁵)SR⁷⁷⁷⁵, —O(C═O)OR⁷⁵, —O(C═O)NR⁷⁵R⁸⁵, —O(C═O)SR⁷⁵, —S(C═O)OR⁷⁵, —S(C═O)NR⁷⁵R⁸⁵, —S(C═O)SR⁷⁵, —NR⁷⁵(C═O)NR⁷⁷⁵R⁸⁵, or —NR⁷⁵(C═S)NR⁷⁷⁵R⁸⁵; in the case of —NR⁷⁵R⁸⁵(R⁹⁵)ₙ₆, R⁷⁵ and R⁸⁵ taken together with the nitrogen atom to which they are attached form a 3-10 membered heterocyclic saturated ring, or heterocyclic unsaturated ring, any of which is optionally substituted with one or more independent halo, cyano, hydroxy, nitro, C₁₋₁₀alkoxy, —SO₂N^{R6}R⁸⁶ or —NR⁷⁶R⁸⁶ substituents;
R^{4a}, R^{4b}, R^{4c}, R^{5a}, R^{5b} and R^{5c} are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷, —SO₂NR⁷⁷R⁸⁷ or —NR⁷⁷R⁸⁷ substituents; or aryl-C₁₋₁₀alkyl, aryl-C₂₋₁₀alkenyl, or aryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷R⁸⁷, —SO₂NR⁷⁷R⁸⁷ or —NR⁷⁷R⁸⁷ substituents; or mono(C₁₋₆alkyl)amino C₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷R⁸⁷, —SO₂NR⁷⁷R⁸⁷ or —NR⁷⁷R⁸⁷ substituents; or R^{4a} with R^{5a}, or R^{4b} with R^{5b}, or R^{4c} with R^{5C}, taken together with the respective carbon atom to which they are attached, form a carbonyl or 3-10 membered saturated or unsaturated monocyclic or polycyclic ring, wherein said ring is optionally substituted with R⁶⁹; or R^{4a} with R^{5a}, or R^{4b} with R^{5b}, or R^{4c} with R^{5c}, taken together with the respective carbon atom to which they are attached, form a 3-10 membered saturated or unsaturated monoheterocyclic or polyheterocyclic ring, wherein said ring is optionally substituted with R⁶⁹;
R^{6a}, R^{6b}, R⁶⁶, R⁶⁷, R⁶⁸, and R⁶⁹ are each independently hydrogen, halo, —OR⁷⁷—SH, —NR⁷⁷R⁸⁸(R⁹⁸)ₙ₇, —CO₂R⁷⁸, —CONR⁷⁸R⁸⁸, —NO₂, —CN, —S(O)ₙ₇R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₀₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸ or —NR⁷⁷⁸R⁸⁸⁸ substituents; or aryl-C₀₋₁₀alkyl, aryl-C₂₋₁₀alkenyl, or aryl-C₂₋₁₀alkynyl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —OR⁷⁷, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or mono(C₁₋₆alkyl)amino C₁₋₆alkyl, di(C₁₋₆alkyl)aminoC₁₋₆alkyl, mono(aryl)amino C₁₋₆alkyl, di(aryl)aminoC₁₋₆alkyl, —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄ alkoxycarbonyl, —CONR⁷⁷⁸R⁸⁸⁸, —SO₂NR⁷⁷⁸R⁸⁸⁸ or —NR⁷⁷⁸R⁸⁸⁸ substituents; or in the case of —NR⁷⁸R⁸⁸(R⁹⁸)ₙ₇, R⁷⁸ and R⁸⁸ taken together with the nitrogen atom to which they are attached form a 3-10 membered mono or polycyclic saturated ring, mono or polycyclic unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents;
R⁷, R⁷¹, R⁷², R⁷³, R⁷⁴, R⁷⁵, R⁷⁷⁵, R⁷⁷⁷⁵, R⁷⁶, R⁷⁷, R⁷⁸, R⁷⁷⁸, R⁸, R⁸¹, R⁸², R⁸³, R⁸⁴, R⁸⁵, R⁸⁶, R⁸⁷, R⁸⁸, R⁸⁸⁸, R⁹, R⁹⁵ and R⁹⁸ are each independently hydrogen, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynmyl, C₁₋₁₀alkylthioC₁₋₁₀alkyl, C₁₋₁₀alkylthio C₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl) or —N(C₀₋₄alkyl) (C₀₋₄alkyl) substituents; aryl-C₀₋₁₀alkyl, aryl-C₂₋₁₀alkenyl, or aryl-C₂₋₁₀alkynyl, any of which is optionally substituted with one or more independent halo, cyano, nitro, —O(C₀₋₄ alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl) or —N(C₀₋₄alkyl)(C₀₋₄alkyl) substituents; or hetaryl-C₀₋₁₀alkyl, hetaryl-C₂₋₁₀alkenyl, or hetaryl-C₂₋₁₀alkynyl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₄alkyl), —SO₂N(C₀₋₄alkyl)(C₀₋₄alkyl) or —N(C₀₋₄alkyl) (C₀₋₄alkyl) substituents; or mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄ alkyl)(C₀₋₄alkyl), —SO₂N(C₀₋₄ alkyl)(C₀₋₄alkyl) or —N(C₀₋₄alkyl) (C₀₋₄alkyl) substituents; and
n1, n2, n3, n4, n5, n6, and n7 are each independently equal to 0, 1 or 2.

8. The composition of claim 2 wherein the CYP26 inhibitor is a compound of formula (IX): 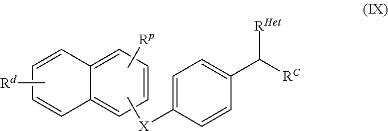
or an isomer, salt, solvate, chemically protected form or prodrug thereof,
wherein X is selected from O, S, NH or CH₂;
R^{d} and R^{P} are optional naphthyl group substituents;
R^{Het} is imidazolyl, triazolyl or pyridyl; and
R^{c} is C₁₋₄alkyl substituted by a group selected from: hydroxy, amino, amido, carboxy, C₁₋₇alkyl ester, C₅₋₇ aryl-C₁₋₂ alkyl ester, sulfonamino, sulfinamino, hydroxamino and tetrazolyl.

9. The composition of claim 2 wherein the CYP26 inhibitor is talarozole.

10. A method for treating a fibrotic or dysproliferative disease or condition in subject in need thereof comprising administering to the subject a therapeutically effective amount of the composition of claim 1, wherein said therapeutically effective amount suppresses fibrosis or the growth of dysproliferative cells in vivo.

11. A method for treating a fibrotic or dysproliferative disease or condition in subject in need thereof comprising administering to the subject a therapeutically effective amount of the composition of claim 2, wherein said therapeutically effective amount suppresses fibrosis or the growth of dysproliferative cells in vivo.

12. The method of claim 10 wherein the dysproliferative disease is cancer.

13. The method of claim 11 wherein the dysproliferative disease is cancer.

14. The method of claim 10 wherein the fibrotic disease or condition is skin fibrosis, acne, psoriasis, ichthyosis, skin photoaging, skin chronoaging, skin wrinkling, skin sagging, skin thickening or skin thinning.

15. The method of claim 11 wherein the fibrotic disease or condition is skin fibrosis, acne, psoriasis, ichthyosis, skin photoaging, skin chronoaging, skin wrinkling, skin sagging, skin thickening or skin thinning.

16. The method of claim 10 wherein the composition is administered topically.

17. The method of claim 11 wherein the composition is administered topically.

18. The method of claim 12 wherein the cancer is breast cancer.

19. The method of claim 13 wherein the cancer is breast cancer.
